(19) United States
(12) Patent Application Publication
(10) Pub. No.: US 2022/0160901 A1
(43) Pub. Date: May 26, 2022

(54) Stimuli-Responsive Compositions, Imaging Systems, and Methods for Using the Same for Biomedical Applications

(71) Applicant: The Regents of the University of California, Oakland, CA (US)

(72) Inventors: Jeffrey I. Zink, Sherman Oaks, CA (US); Holden H. Wu, Los Angeles, CA (US); Chi-An Cheng, Los Angeles, CA (US); Wei Chen, Los Angeles, CA (US); Tian Deng, Los Angeles, CA (US); Navnita Kumar, Los Angeles, CA (US); Le Zhang, Los Angeles, CA (US)

(21) Appl. No.: 17/439,161

(22) PCT Filed: Mar. 16, 2020
(86) PCT No.: PCT/US2020/022993

§ 371 Date: Sep. 14, 2021

Related U.S. Application Data
(60) Provisional application No. 62/818,636, filed on Mar. 14, 2019.

(51) Int. Cl. A61K 49/18; A61B 5/055; C01B 33/18; A61B 5/0515

(52) U.S. Cl. CPC A61K 49/186; A61B 5/055; C01B 33/18; C01P 2004/62; C01P 2006/42; C01P 2004/64; A61B 5/0515

(57) ABSTRACT

The present disclosure provides stimuli-responsive particles, methods of preparing stimuli-responsive particles, and methods of using the stimuli-response particles. Unlike conventional platforms, (e.g., polymers, liposomes, dendrimers) the particles of the present disclosure have precise size control of the particle diameter, high uniformity, high stability, high active agent uptake capacity, minimal premature active agent leakage, biocompatibility, and biodegradability. Additionally, the present disclosure provides magnetic resonance imaging (MRI) systems and methods of using the MRI systems in combination with the stimuli-responsive particles described herein.

### CROSS REFERENCE TO RELATED APPLICATIONS

This patent application claims the benefit of and priority to U.S. Provisional Patent Application Ser. No. 62/818,636 filed Mar. 14, 2019, which is incorporated herein by reference in its entirety.

### BACKGROUND

Non-invasive imaging is an important technology for theranostics (e.g., integrated diagnosis and therapy). Compared to other imaging modalities (e.g., X-ray, computed tomography, ultrasound, positron emission tomography, optical), magnetic resonance imaging (MRI) provides an especially powerful suite of different in vivo contrast mechanisms for precise identification and characterization of diseased tissues. In addition, MRI does not involve ionizing radiation, supports flexible imaging orientations, and achieves 3D coverage even for tissues deep in the body.

As a result, MRI is increasingly being used in clinical practice to guide therapies, such as thermal ablation using high-intensity focused ultrasound (HIFU) or near-infrared laser. MRI-guided targeted agent delivery is also being developed. Nanoparticle technology has opened up the possibility of delivering materials to specific sites of interest at a cellular level, enhancing imaging contrast, or enabling controlled release of encapsulated theranostic agents. In particular, the combination of MRI with nanoparticle technology provides a compelling platform to realize image-guided theranostics. However, current nanoparticle platforms (e.g., polymers, liposomes, dendrimers) still face critical challenges in size control, stability, biocompatibility, agent uptake/release capacity, premature agent leakage, and MRI detection sensitivity and specificity.

### SUMMARY OF THE DISCLOSURE

Some embodiments of the present disclosure provide a stimuli-responsive composition comprising a particle having an outer surface and a plurality of pores that are sized to receive one or more active agent therein. The particle may be silica, and particularly a silica nanoparticle. The stimuli-responsive composition may have a plurality of capping agents coupled to the outer surface and arranged to cover at least a fraction of the plurality of pores. The capping agents may have a first physical-chemical state that prevents the active agents from being released from at least a portion of the plurality of pores and a second physical-chemical state that allows the passage of the active agents from the plurality of pores. The capping agents may be characterized as having a structure that is transformable from the first physical-chemical state to the second physical-chemical state in response to an external stimulus applied to the capping agents in an effective amount. In some embodiments, the capping agents are selected from one or more of a polymer having a polyether backbone, a thermo-responsive polymer having reversible hydrophilicity, a mechano-responsive polymer configured to vibrate and/or translate upon application of an effective amount of external stimuli, a polymer with bonds that are mechano-responsive and can also be ruptured (e.g., irreversibly transformed) by an effective amount of the external stimulus, a compound having an alkyl-azo moiety positioned along the length of the capping agent, and a macrocyclic molecule that is coupled to the silica particle through a linking agent where the macrocyclic molecule is non-covalently bound to the linking agent.

In further embodiments, a method of delivering an active agent to a region of interest in a subject is provided. The method includes administering a stimuli-responsive composition to the region of interest of the subject, where the stimuli-responsive composition comprises silica particles having an outer surface and a plurality of pores that are sized to receive one or more active agent therein; and a plurality of capping agents coupled to the outer surface and arranged to cover at least a fraction of the plurality of pores. The capping agents may have a first physical-chemical state that prevents the active agents from being released from at least a portion of the plurality of pores and a second physical-chemical state that allows the passage of the active agents from the plurality of pores. The method further includes applying an external stimulus to the capping agents in an effective amount to transform the capping agents from the first physical-chemical state to the second physical-chemical state to allow the passage of the active agent to the region of interest in the subject. In some forms, the agent comprises a therapeutic agent and the external stimulus is applied to the capping agent for a sufficient dosage or duration to induce a therapeutic effect in the subject. Alternatively or additionally, the active agent comprises a contrast agent and the external stimulus is applied to the capping agent for a sufficient dosage or duration to improve the visibility of the region of interest in the subject during a medical imaging procedure.

In some embodiments, a method for producing a magnetic resonance image of a subject with enhanced contrast and reduced background signal is provided. The method may include administering a stimuli-responsive composition to a region of interest in the subject. The stimuli-responsive composition may comprise a plurality of particles having a structure that is transformable from a first state to a second state in response to an external stimulus applied in an effective amount, where the second state enhances magnetic resonance contrast within the region of interest relative to the first state. The method further includes applying an external stimulus to at least a portion of the particles for a first duration to alter the particles from a first state to a second state. A first set of magnetic resonance data may then be acquired from the region of interest during the first duration when the particles are in the second state. The method further includes ceasing the application of the external stimulus for a second duration to allow the particles to transform from the second state to the first state. A second set of magnetic resonance data is then acquired from the region of interest during the second duration when the particles are in the first state. The method further includes computing a signal change map from the region of interest having values indicating a difference between the first set of magnetic resonance data and the second set of magnetic resonance data, and generating an image based at least in part on the values from the signal change map.

### DESCRIPTION OF THE DRAWINGS

FIG. 1 is a schematic illustration of an example stimuli-responsive composition and a capping agent formed from a polymeric matrix in accordance with embodiments of the present disclosure.

FIG. 2 is a schematic illustration of an example stimuli-responsive composition and a capping agent formed from a macrocyclic molecule in accordance with embodiments of the present disclosure.

FIG. 3 is a schematic illustration of an example stimuli-responsive composition and a capping agent formed from an ultrasound labile compound or moiety in accordance with embodiments of the present disclosure.

FIG. 4 is a graphical illustration of pores, linking agents, and capping agents coupled to the stimuli-responsive composition of FIG. 3.

FIG. 5 is a schematic illustration of an example stimuli-responsive composition and a capping agent formed from a polymer having a reversible hydrophilicity in accordance with embodiments of the present disclosure.

FIG. 6 is a schematic illustration of an example stimuli-responsive composition having a hollow chamber and superparamagnetic core disposed therein in accordance with embodiments of the present disclosure.

FIG. 7 is a schematic illustration of example stimuli-responsive compositions formed from paramagnetic particles in accordance with embodiments of the present disclosure.

FIG. 8 is a block diagram illustrating an example of an external stimuli activation system that can implement some embodiments of the present disclosure.

FIG. 9 is a is a block diagram of an example of a magnetic resonance imaging (“MRI”) system that can implement the methods in accordance with embodiments of the present disclosure.

FIG. 10 is a flowchart setting forth the steps of a method for generating a contrast enhanced image in accordance with embodiments of the present disclosure.

FIGS. 11(A-B) is a synchronized HIFU sequence applied to a sample region including (a) a warm-up sequence of 24.5 W followed by a periodic HIFU sequence with a frequency of 0.1 Hz (18 W, 5 s on and 5 s off, repeated 10 times); (b) TiW intensity change over time of the sample (region A), phantom (region B), and background (region C) of Gd(DTPA)²⁻ and PNIPAm modified MSNs (Gd-P-MSNs) with HIFU (second from top line), Gd-P-MSNs no HIFU (top line), and Magnevist (Mgv) with HIFU (third from top line).

FIGS. 12(A-F) are exemplary TEM images of mesoporous silica nanoparticles (MSNs) and results of MSN, MSN-APTS, and MSN-PEG particle characterization; (a) is a TEM image of MSNs (left), MSNs-APTS (middle) and MSNs-PEG (right); (b) are Zeta potential values of the MSNs, MSNs-APTS, and MSNs-PEG; (c) is a Fourier transform infrared spectroscopy (FT-IR) graph, (d) is a thermogravimetric analysis (TGA) of MSNs, MSNs-APTS, and MSNs-PEG, respectively; (e) is N₂ adsorption/desorption isotherms of MSNs, MSNs-APTS, MSNs-PEG, and Gd(DTPA)²⁻-loaded MSNs-PEG; and (f) is Dynamic light scattering (DLS) size distribution of MSNs, MSNs-APTS, and MSNs-PEG in deionized H₂O, and MSNs-APTS and MSNs-PEG in PBS.

FIGS. 13(A-D) are exemplary results of the MSN-PEG functionalization; (a) is a graph of release efficiency of Gd(DTPA)²⁻ from MSNs-PEG after 2, 5, 8, 10, or 30 min of treatment with a probe sonicator, in a 37° C. or 50° C. hot water bath, or in a 23° C. water bath. The loading capacity of Gd(DTPA)²⁻ in MSNs-PEG was 25.6%; (b) is a graph of relaxivity (r₁) values of Gd(DTPA)²⁻ loaded MSNs-PEG without (square) and with (circle) ultrasound treatment by the probe sonicator, and free Gd(DTPA)²⁻ (triangle); (c) is T₁ relaxation time of Gd(DTPA)²⁻ loaded MSNs-PEG after 2, 5, 8, 10, and 30 min of treatment with the probe sonicator. Inset shows the corresponding T₁-weighted images; and (d) is a graph of correlation between changes in T₁ relaxation rate with the release efficiency of Gd(DTPA)²⁻.

FIGS. 14(A-D) are exemplary results of HIFU stimulation; (a) is an illustration of the setup of HIFU stimulation during the release study. Gd(DTPA)²⁻ loaded MSNs-PEG were dispersed in the mixture of methyl cellulose and concentrated milk, and filled in a 3 cm-in-depth well (yellow) created in an agarose phantom. The water cooled HIFU transducer was put under the agarose phantom and HIFU beam was focused to a cigar-shaped focal point with dimension of 1×1×7 mm near the center of the sample. The illustration is drawn to scale; (b) is an axial view of Δ (pre−post) T₁-weighted images obtained by subtracting the T₁-weighted images after HIFU stimulation from before HIFU stimulation; (c) is a coronal view of Δ (pre−post) T₁-weighted images obtained by subtracting the T₁-weighted images after HIFU stimulation from before HIFU stimulation; (d) is a sagittal view of Δ (pre−post) T₁-weighted images obtained by subtracting the T₁-weighted images after HIFU stimulation from before HIFU stimulation.

FIGS. 15(A-D) are exemplary MRI results of the MSN-PEG particles; (a) are T₁-weighted images of Gd(DTPA)²⁻ loaded MSNs-PEG in water (sample 1) before (left) and after (middle) HIFU stimulation (1). The Δ T₁-weighted image (pre−post) was also shown (right). Controls included 2: Gd(DTPA)²⁻ loaded MSNs-PEG in water, 3: methyl cellulose gel/milk, 4-6: Gd(DTPA)²⁻ loaded MSNs-PEG in methyl cellulose gel/milk; (b) is a graph of the percentage of T₁-weighted image intensity change with or without the HIFU stimulation. The release efficiency difference was quantified by ICP-OES; (c) is a series of temperature profiles for water and the mixture of methyl cellulose and concentrated milk suspended Gd(DTPA)²⁻ loaded MSNs-PEG during the HIFU stimulation; (d) is a graph of relaxivity (r₁) values of Gd(DTPA)²⁻ loaded MSNs-PEG after HIFU stimulation.

FIG. 16(A-C) are exemplary active agent release results of MSN-PEG particles; (a) is a graph of multiple HIFU stimulation cycles (3 cycles of 1 min) to Gd(DTPA)²⁻ loaded MSNs-PEG. The rectangular boxes show the duration of the HIFU stimulation. T₁-weighted image intensities of Gd(DTPA)²⁻ loaded MSNs-PEG (black) and agarose background (red) are shown; (b) is a graph of the percentage of T₁-weighted image intensity change of Gd(DTPA)²⁻ loaded MSNs-PEG after 1, 3, 5, or 10 min of HIFU stimulation (74 W); (c) is a graph of the percentage intensity change of T₁-weighted image of Gd(DTPA)²⁻ loaded MSNs-PEG after 3 min HIFU stimulation at three different power outputs levels (9 W, 74 W, and 290 W).

FIGS. 17(A-D) are exemplary MALDI-TOF spectra of PEG (Mₙ 2000 Da) capping agents coupled to silica nanoparticles; (a) is a MALDI-TOF spectra without HIFU stimulation; (b) is a MALDI-TOF spectra after 3 cycles of 1 min HIFU stimulation; (c) is a MALF-TOF spectra after 2 cycles of 5 min HIFU stimulation; (d) is a zoomed-in MALDI-TOF spectra of intensity ratios of the peaks around m/z=1000 to m/z=2000 calculated as (the highest intensity of the peak around m/z 1000/the highest intensity of the peak around m/z=2000) were determined to be 11%, 20%, and 71% for (a), (b), and (c), respectively.

FIGS. 18(A-E) are exemplary MRI contrast enhancement data and images using different HIFU parameters; (a) is a graph of the percentage change of T₁-weighted image intensity of Gd(DTPA)²⁻-loaded MSNs-PEG after 3, 5, or 10 min of HIFU stimulation (74 W), where the insets show the corresponding temperature increase for each experiment with different HIFU stimulation parameters; (b) is a graph of the percentage change of T₁-weighted image intensity of Gd(DTPA)²⁻-loaded MSNs-PEG after 3 min of HIFU stimulation at three different power levels (9 W, 74 W, and 290 W), where the insets show the corresponding temperature increase for each experiment with different HIFU stimulation parameters; (c) is Δ T₁-weighted image of Gd(DTPA)²⁻-loaded MSNs-PEG after different time durations or power levels of HIFU stimulation; (d) is a graph of positive correlations between T₁ relaxation times with the release efficiencies of Gd(DTPA)²⁻ after different time durations; and (e) is a graph of positive correlations between T₁ relaxation times with the release efficiencies of Gd(DTPA)²⁻ after different power levels of HIFU stimulation.

FIGS. 19(A-D) are exemplary images and data corresponding to HIFU-activated Magnevist release ex vivo in chicken breast and the controllable MRI contrast changes; (a) is a Δ T₁-weighted image after each cycle of HIFU stimulation (3 min, 2.5 MHz, 8 W) of the chicken breast injected with gel only (chicken 1, the dimension is shown in the left photo). Gel (HIFU) indicates the gel injection site stimulated with HIFU; (b) is a graph showing the percentage change of T₁-weighted image intensity after each cycle of HIFU stimulation of the chicken 1 background, gel (HIFU), chicken 2 background, sample (w/o HIFU), and sample (HIFU) (pre−post HIFU 1 means T₁-weighted image intensity before HIFU stimulation minus T₁-weighted image intensity after the first cycle of HIFU stimulation. Total number of cycles=2 for chicken 1, and 3 for chicken 2); (c) is a T₁-weighted image before HIFU stimulation of the other chicken breast (chicken 2) injected with Gd(DTPA)²⁻-loaded MSNs-PEG mixed in methyl cellulose gel. Sample (HIFU) indicates the sample injection site stimulated with HIFU. Sample (w/o HIFU) indicates the sample injection site without HIFU stimulation; and (d) is a Δ T₁-weighted image after each cycle of HIFU stimulation (3 min, 8 W) of chicken 2 injected with Gd(DTPA)²⁻-loaded MSNs-PEG mixed in gel.

FIG. 20 is an example synthesis route of post-grafting linking agents on particles in accordance with embodiments of the present disclosure.

FIG. 21 is an example synthesis route of coupling a thermo-responsive polymer on a particle in accordance with embodiments of the present disclosure.

FIG. 22 are exemplary TEM images of unmodified MSN (left) and PNIPAm-MSN (right).

FIG. 23(A-B) are exemplary graphs of the hydrodynamic diameter distribution of PNIPAm-MSN at 30° C. (a) and 40° C. (b).

FIG. 24 is an example graph of the Zeta-potential (mV) of MSN, NH₂-MSN, Gd-MSN, and PNIPAm-MSN, respectively.

FIG. 25 is an example synthesis route of Magnevist (Gd-DTPA) loaded PNIPAm-MSN.

FIG. 26 is an exemplary plot of T₁ relaxation time of Gd-DTPA loaded PNIPAm-MSN and Magnevist control. Increase T₁ indicate continuous Magnevist release after HIFU triggers.

FIG. 27 illustrates a plot and Tt images acquired of the stimuli-responsive particles according to some embodiments of the present disclosure during and after HIFU stimulation. The plot (left) is of the brightness of sample area on T₁-weighted images verses HIFU modulation step. Tt-weighted images (right) illustrate the brightness of Gd-P-MSN decreases during HIFU, and the brightness will return to after HIFU, indicating a reversible MRI contrast change caused by HIFU.

FIG. 28 is an exemplary plot of T₁ relaxation time (ms) of samples with different ratios of Gd-DTPA to PNIPAm. All samples showed reversible T₁ changes after HIFU.

FIG. 29 is an exemplary graph of Gd-P-MSNs with different Gd/PNIPAm mole ratio and Magnevist control (Mgv) and PNIPAm modified MSNs (P-MSNs) control.

FIG. 30 is an exemplary plot of T₁ relaxation time of Gd-P-MSN and Magnevist control. Gd-P-MSN with Gd-DTPA modified in pores did not show much T₁ change during HIFU.

FIG. 31(A-B) illustrates (a) the effect of ultrasonication or HIFU stimulation on transverse relaxivity (r₂). r₂ of fluorescein-loaded MNP@MSN-AMA-CD in deionized water before and after stimulation with a probe sonicator (10 min) or HIFU (3 min, 74 W) are shown; and (b) The effect of DOX amount in the pores on r₂. r₂ of DOX-loaded MNP@MSN-AMA-CD in PBS with 0, 12.5, 18.4, or 20.6 μM of DOX concentration loaded in the pores are shown.

FIG. 32(A-D) illustrate (a) time-dependent release profile of DOX over a period of 27 h after 1, 5, or 10 min of HIFU stimulation (1 MHz, 74 W) and (b) R₂ quantified immediately after HIFU stimulations with different exposure lengths. Inset in (b) shows the corresponding T₂ maps. Associations between R₂ from (b) and the release efficiencies of DOX measured at 1.6 and 27 h after those HIFU stimulations from (a) were shown in (c) and (d), respectively.

FIG. 33 is an exemplary chart of T₁ and T₂ changes of core-shell MSNs capped with PEG before and after trigger with the probe sonicator. After the ultrasound (US) trigger, the decrease in both T₁ and T₂ were observed for the nanoparticles with Magnevist loading.

FIG. 34 is an exemplary chart of T₁ and T₂ intensity changes in weighted images of core-shell MSNs capped with PEG before and after HIFU trigger. After HIFU stimulation, both T₁ and T₂ intensity changes were observed for the nanoparticles with Magnevist loading.

FIG. 35(A-C) are schematic illustrations and TEM images of an example stimuli-responsive compositions having a hollow chamber and a superparamagnetic particle disposed therein in accordance with embodiments of the present disclosure; (a) is a schematic illustration of the synthesis procedures of superparamagnetic core encapsulated hollow mesoporous silica nanoparticles (HMSNs); (b) is a transmission electron microscope image of non-porous silica coated superparamagnetic core; and (c) is a superparamagnetic core encapsulated HMSNs.

FIG. 36 is a schematic illustration of energy levels of iron d-orbitals and their occupancies by 6 electrons at low and high temperatures. The changes in the magnetization as a function of the temperature are shown on the right.

FIG. 37 is a graph of magnetic susceptibility of Fe(Me-bik)₃t have any build item except for a 6/2 text editor that i received on Saturday9 (BF₄)₂.25H₂O as a function of temperature obtained by using Evan's Method.

FIG. 38 is a graph of T₁ values for Fe(Me-bik)₃](BF₄)₂.25H₂O at various concentration relative to that of water as a function of temperature.

FIG. 39 is a graph of T₂ values for Fe(Me-bik)₃](BF₄)₂.25H₂O at various concentration relative to that of water as a function of temperature.

FIG. 40 are MRI images (T₁ and T₂ weighted) of Fe(Me-bik)₃](BF₄)₂.25H₂O sample at various concentrations taken at room temperature (RT) and at 70° C. (HT).

FIG. 41 is a schematic illustration of the locations of samples characterized in the MRI images of FIG. 40.

FIGS. 42(A-B) are images of ultrasmall iron oxide nanoparticles (USIONs); (a) is an image of USIONs with an average diameter of 2.8±0.3 nm; (b) is an image of USIONs with an average diameter of 3.6±0.4 nm.

FIGS. 43A-43B are a Fourier transform infrared (FT-IR) spectra of USIONs; (a) is a FT-IR before the surface capping agents (oleic acid and oleylamine) stripped, and aminoazobenzene or carboxyazobenzene conjugated USIONs; (b) is a FT-IR after the surface capping agents (oleic acid and oleylamine) stripped, and aminoazobenzene or carboxyazobenzene conjugated USIONs

FIG. 44 is a graph of the percentage decrease of T₁ values of different concentrations iron(II) complexes with respect to water at high temperature compared to room temperature using NMR spectroscopy.

FIG. 45A is a graph of the percentage decrease of T₁ values of iron(II) complexes of different concentrations with respect to gel at high temperature compared to room temperature using MRI during cycle 1.

FIG. 45B is a graph of the percentage decrease of T1 values of iron(II) complexes of different concentrations with respect to gel at high temperature compared to room temperature using MRI during cycle 2.

FIG. 46A is a graph of the percentage decrease of T₁ values of iron(II) complexes of different concentrations with respect to water at high temperature compared to room temperature using MRI during cycle 1.

FIG. 46B is a graph of the percentage decrease of T₁ values of iron(II) complexes of different concentrations with respect to water at high temperature compared to room temperature using MRI during cycle 2.

FIG. 47 is a graph of T₁ values of different concentrations of loaded iron(II) complexes within phosphonate-nanoparticles, unloaded phosphonate-nanoparticles (capped and uncapped) with respect to water at high temperature compared to room temperature using NMR spectroscopy.

FIG. 48 is the percentage decrease of T₁ values of different concentrations of loaded iron(II) complexes within phosphonate-nanoparticles, unloaded phosphonate-nanoparticles (capped and uncapped) with respect to water at high temperature compared to room temperature using NMR spectroscopy. Reversibility of the systems are shown by performing cycle 1 and cycle 2.

FIG. 49 are T₁ weighted images of different concentrations of iron(II) complex, loaded iron(II) complexes within phosphonate-nanoparticles, unloaded phosphonate-nanoparticles (capped and uncapped) with respect to gel at high temperature compared to room temperature using MRI.

FIG. 50 is a graph of T₁ values of different concentrations of loaded iron(II) complexes within phosphonate-nanoparticles, unloaded phosphonate-nanoparticles (capped and uncapped) with respect to gel at high temperature compared to room temperature using MRI.

FIG. 51A is a graph of the percentage decrease of T₁ values of different concentrations of loaded iron(II) complexes within phosphonate-nanoparticles, unloaded phosphonate-nanoparticles (capped and uncapped) with respect to gel at higher temperatures compared to room temperature using MRI during cycle 1.

FIG. 51B is a graph of the percentage decrease of T₁ values of different concentrations of loaded iron(II) complexes within phosphonate-nanoparticles, unloaded phosphonate-nanoparticles (capped and uncapped) with respect to gel at higher temperatures compared to room temperature using MRI during cycle 2.

FIG. 52 is a schematic illustration of a stimuli-responsive composition according to some embodiments of the present disclosure.

FIG. 53 is an example synthetic method for producing the stimuli-responsive composition of FIG. 52 in accordance with some embodiments of the present disclosure.

FIG. 54 plots Cartesian T₁-weighted intensity of a EO/PO/EO-Gd-MSNs before (pre), during, and after (post) HIFU of 50% (50 W) and 70% (98 W) amplitude.

FIG. 55 is T₁-weighted image (left) and HIFU modulation enhancement map (MEM) (right) of 25R2-Gd-MSNs, trial number R5.

FIGS. 56(A-D) is an example synthetic method for producing Gd-P-MSN: (a) bare MSNs, (b) amine modified MSNs (NH₂-MSNs), (c) Gd-DTPA modified MSNs (Gd-MSNs), (d) Gd-P-MSNs.

FIGS. 57(A-F) illustrates temperature (bottom lines in a-c) and TiW intensity (top line in a-c) changes of (a) Gd-P-MSNs with HIFU (b) Gd-P-MSNs no HIFU and (c) Mgv with HIFU. All values were average of 9 pixels around the HIFU focal point. (d)-(f) are Fourier transform spectra of TiW intensity changes vs. time of one pixel on the HIFU focal point in (a)-(c). DC (0 Hz) peak intensity in each spectrum was normalized to 1. The area under the 0.1 Hz peak (HIFU modulation frequency in this experiment) in (d) is much larger than that in (e) and (f).

FIGS. 58(A-F) illustrates TiW images, modulation enhancement maps (MEMs) and contrast difference % (CD %) of samples and controls. In (a) through (e), the edge of the agarose phantom is delineated with the outer dotted circle, and the sample/control region is delineated by an inner dotted circle. (a) and (b) are TiW images before periodic HIFU of Mgv (Mgv TiW before HIFU) and Gd-P-MSNs (Gd-P-MSNs TiW before HIFU). The black spot in (a) and (b) are from a temperature probe. (c)-(e) are MEMs of Mgv with HIFU, Gd-P-MSNs no HIFU and Gd-P-MSNs with HIFU. (f) CD % of (a)-(e).

FIGS. 59(A-E) illustrates (a) an example workflow for characterizing the HIFU-stimulated DOX delivery and its therapeutic efficacy in PANC-1 cells via MRI. (b) T₂ maps of PANC-1 treated with DOX-MNP@MSNs-AMA-CD after different HIFU stimulation times. (c) R₂ and (d) cell viability of PANC-1 treated with DOX-MNP@MSNs-AMA-CD, MNP@MSNs-AMA-CD, or the equivalent amount of free DOX to DOX-MNP@MSNs-AMA-CD (positive control), and cells only (negative control) after each HIFU stimulation. Cell viability was analyzed by a CCK-8 assay after 18 h growth post HIFU stimulation. Data in (c) and (d) are displayed as the mean (color bar)±standard deviation (black brackets) of three independent experiments. The association between R₂ and cell viability is shown in (e).

### DETAILED DESCRIPTION

In some embodiments, the present disclosure provides stimuli-responsive particles, methods of preparing stimuli-responsive particles, and methods of using the stimuli-response particles. Unlike conventional platforms (e.g., polymers, liposomes, dendrimers), the particles of the present disclosure have precise size control, high uniformity, high stability, high active agent uptake capacity, minimal premature active agent leakage, biocompatibility, and biodegradability. Additionally, the present disclosure provides imaging systems and methods, such as magnetic resonance imaging (MRI) systems and methods of using the MRI systems in combination with the stimuli-responsive particles described herein.

### I. Particles for the Stimuli-Responsive Compositions

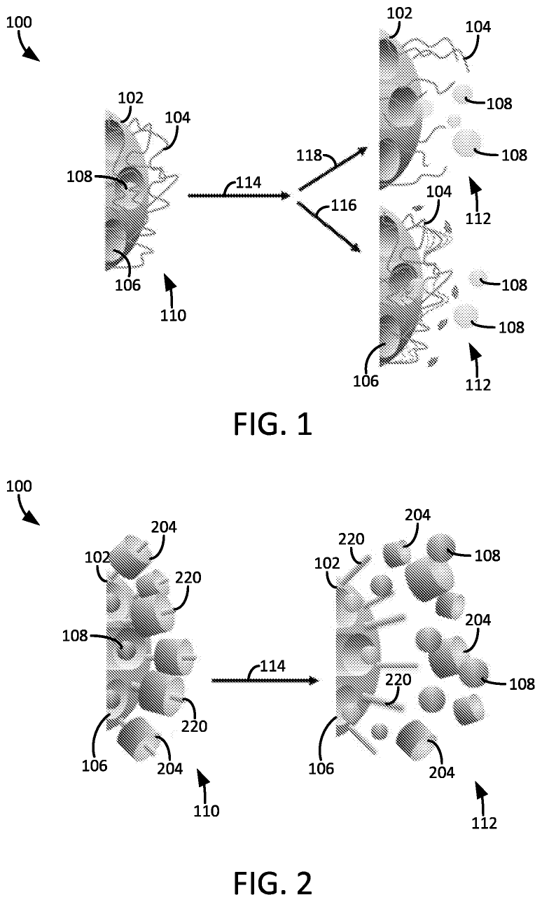

Referring to FIG. 1, the stimuli-responsive compositions 100 of the present disclosure includes particles 102 that define a body, scaffold, or shape having an outer surface and an inner volume. In some embodiments, the particles 102 can define a porous structure. For example, the particles 102 may have pores 106. The terms “porous” and “porosity” are generally used to describe a structure having a connected network of pores or void spaces (which can, for example, be openings, interstitial spaces or other channels) throughout its volume. The term porosity is a measure of void spaces in a material, and is a fraction of volume of voids over the total volume, as a percentage between 0 and 100% (or between 0 and 1). In some embodiments, a portion of the pores 106 in the particles 102 are sized to receive one or more active agent 108 therein. The pores may be partially or completely interconnected, however, this is not required.

In some embodiments, the stimuli-responsive compositions 100 include particles 102 formed from one or more metal oxide, mixed metal oxide, semi-metal oxide, mixed semi-metal oxides, and combinations thereof. Exemplary particles 102 include, but are not limited to, silicon dioxide particles (e.g., silica) and, more particularly, silicon dioxide nanoparticles. In some embodiments, the stimuli-responsive compositions 100 include particles 102 having a body that consists essentially of silica or consists of silica. Silica particles offer several advantages over conventional platforms at least due to silica's tunable surface area, pore volume, high biocompatibility, high cellular internalization efficiency, and facile surface functionalization.

In some embodiments, the particles 102 in the stimuli-responsive composition 100 include nanoparticles having at least one dimension (e.g., length, height, diameter) that is on a nanometer scale. In some embodiments, the dimension is on a nanometer scale that may range from 1 nm to approximately 1000 nm, or more. In other embodiments, the particles 102 have a diameter of at least 5 nm, at least 10 nm, at least 15 nm, at least 20 nm, at least 25 nm, at least 30, nm, at least 35 nm, at least 40 nm, at least 45 nm, at least 50 nm, at least 55 nm, at least 60 nm, at least 65 nm, at least 70 nm, at least 75 nm, at least 80 nm, at least 85 nm, at least 90 nm, at least 95 nm, or to at least 100 nm. In other embodiments, the particles 102 have a diameter of less than 100 nm, less than 150 nm, less than 200 nm, less than 250 nm, less than 300 nm, less than 350 nm, less than 400 nm, less than 450 nm, less than 500, nm less than 550 nm, less than 600 nm, less than 650 nm, less than 670 nm, less than 750 nm, less than 800 nm, less than 850 nm, less than 900 nm, less than 950 nm, less than 1000 nm, In some embodiments, the particles 102 have a diameter from 1 nm to 300 nm. In other embodiments, the particles 102 have a diameter that is between 1 nm to 100 nm.

In some embodiments, the particles 102 in the stimuli-responsive composition 100 can be configured to have a tunable porosity. For example, in some embodiments, the particles 102 can have a porosity of at least 5%, at least 10%, at least 15%, at least 20%, at least 25%, at least 30%, at least 35%, at least 40%, at least 45%, at least 50%, at least 55%, at least 60%, at least 65%, at least 70%, or to at least 75%. In some embodiments, the porosity of the particles 102 is less than 80%, less than 85%, less than 90%, less than 91%, less than 92%, less than 93%, less than 94%, less than 95%, less than 96%, less than 97%, less than 98%, less than 99%, but excluding 100%. In some embodiments, the porosity ranges from 50% to 99%, or from 60% to 99%, or from 70% to 99%. The pore size and total porosity values can be quantified using conventional methods and models known to those of skill in the art. For example, the pore size and porosity can be measured by standardized techniques, such as mercury porosimetry and nitrogen adsorption, or methods described in the Examples.

The pores can be adapted to have any shape, e.g., circular, elliptical, polygonal, or amorphous. The particles can be adapted to have pores having a pore size of 2 nm to 75 nm, 2 nm to 50 nm, 2 nm to 30 nm, or 2 nm to 15 nm. The term “pore size” may refer to a dimension of the pores. In some embodiments, the particles can be adapted to have pores having a pore size of 2 nm, 3 nm, 4 nm, 5 nm, 6 nm, 7 nm, 8 nm, 9 nm, and 10 nm. For example, the pore size can refer to the longest dimension of a pore, e.g., a diameter or a pore having a circular cross section, or the length of the longest cross-sectional chord that can be constructed across a pore having non-circular cross-section.

In some embodiments, the surface area of the particles 102 ranges from 50 m²/g to 1200 m²/g. For example, in some embodiments, the surface area of the particles 102 is at least 100 m²/g, or is at least 150 m²/g, or is at least 200 m²/g, or is at least 250 m²/g, or is at least 300 m²/g, or is at least 350 m²/g, or is at least 400 m²/g, or is at least 450 m²/g, or is at least 500 m²/g, or is at least 550 m²/g, or is at least 600 m²/g, or is at least 650 m²/g, or is at least 700 m²/g. In some embodiments, the particles 102 have a surface area of less than 750 m²/g, or less than 800 m²/g, or less than 850 m²/g, or less than 900 m²/g, or less than 950 m²/g, or less than 1000 m²/g, or less than 1050 m²/g, or less than 1100 m²/g, or less than 1150 m²/g, or less than 1200 m²/g. The surface area values can be quantified using conventional methods and models known to those of skill in the art. For example, the surface area can be measured by standardized techniques, such as adsorption techniques (Brunauer-Emmett-Teller adsorption method), or methods described in the Examples.

In some embodiments, the pores 106 may be sized to receive one or more active agent therein. The active agent may be mixed, dispersed, or suspended in the stimuli-responsive composition such that it becomes distributed or embedded in the pores of the particles. The term “active agent” as used herein may refer to a chemical moiety or compound that belongs to a chemical class including, but not limited to, polypeptides, nucleic acids, saccharides, lipids, small molecules, biocompatible metals, biocompatible metal oxides nanoparticles, chelates, and combinations thereof.

The term “biocompatible” as used herein may refer to a composition that does not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death (e.g., less than 10% or 5%), and/or their administration in vivo does not induce significant inflammation or other such adverse effects.

In certain embodiments, the active agent comprises a therapeutic agent. The term “therapeutic agent” is an art recognized term and refers to any chemical moiety or compound that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

In other embodiments, the active agent may comprise a contrast agent. The term “contrast agent” is an art recognized term that refers to a compound or chemical moiety that improves the visibility of an internal body structure during an imaging procedure, such as an x-ray imaging procedure (e.g., high-energy radiation), an ultrasound imaging procedure (e.g., high-energy sound waves), or a radio wave imaging procedure. Contrast agents suitable for use in the present disclosure include chemical moieties or compounds that improve the visibility of internal body structures during imaging procedures relating to, without limitation, magnetic resonance imaging (MRI), computed tomography (CT), projection radiography, fluoroscopy, X-ray imaging, and ultrasound imaging. Exemplary contrast agents include gadolinium-based MRI agents, iodinated radiocontrast agents, iron oxide contrast agents, and fluorescent compounds or moieties. In some embodiments, the contrast agent comprises a compound or chemical moiety that alters the relaxivity (e.g., shorten or lengthen relaxation times) of nuclei within the region of interest during magnetic resonance imaging (MRI). Non-limiting examples of gadolinium-based MRI agents include, but are not limited to, gadopentetate (Magnevist), gadoterate (Dotarem, Clariscan), gadodiamide (Omniscan), gadobenate (MultiHance), gadoteridol (ProHance), gadoversetamide (OptiMARK), gadobutrol (Gadavist), gadopentetic acid dimeglumine (Magnetol), gadofosveset (Ablavar), gadocoletic acid, gadomelitol, gadomer 17, gadoxetic acid (Eovist).

### II. Capping Agents for the Stimuli-Responsive Compositions

In some embodiments, the stimuli-responsive composition 100 includes one or more capping agent 104 coupled to a surface on the particle 102. In some embodiments, the capping agents 104 are arranged on the external surface of the particle 102 to regulate the release of active agents 108 from the pores 106, for example, by covering a sufficient fraction of an opening to the pores 106 to prevent the release of the active agents 108 from within the pore 106. The capping agents 104 may be coupled to inner surfaces of the pores 106 and outer surfaces of the particles 102.

In some embodiments, the capping agents 104 comprise a structure that is transformable from a first physical-chemical state 110 to a second physical-chemical state 112, where the first physical-chemical state 110 prevents the active agents 108 from being released from the pores 106, and the second physical-chemical state 112 allows the passage of the active agents 108 from the pores 106 to a volume outside of the particle 102.

As indicated by process block 114, the capping agents 104 may be transformed from the first physical-chemical state 110 to the second physical-chemical state 112 in response to an external stimulus or trigger applied in an effective amount.

As used herein, applying an “effective amount” may refer to exposing the capping agents 104 to the external stimuli for a duration or dosage that is sufficient to elicit or influence the capping agents 104 to transform from the first physical-chemical state 110 to the second physical-chemical state 112. Without being bound to any particular theory, it is contemplated that the release of the active agents 108 from the pores 106 may be the result of two mechanisms, which may be controlled based on the amount of external stimuli applied to the capping agents 104.

It is contemplated that the first mechanism, as shown through process block 118, includes applying the external stimuli to the capping agents 104 in an amount sufficient to irreversibly transform the capping agents 104 from the first physical-chemical state 110 to the second physical-chemical state 112. Irreversible transformation may be induced by applying the external stimuli in an amount sufficient to rupture at least a portion of the bonds (e.g., covalent or non-covalent) in capping agents 104. Rupturing the bonds may be induced, for example, by applying ultrasound in an amount sufficient to induce cavitation or shock waves in a region around the capping agents 104. Cavitation may cause a rapid compression with subsequent expansion of the liquid, where on a molecular level, implies a rapid motion of small molecules (e.g., solvent molecules, active agents) to which the polymer in the solvent cannot follow. Thus, friction is generated, strain is increased, and eventually, bond rupture may occur at a point along the capping agents length, (e.g., the midpoint).

It is contemplated that the second mechanism, as shown through process block 116, includes applying the external stimuli to the capping agents 104 in an amount sufficient to reversibly transform the capping agents 104 from the first physical-chemical state 110 to the second physical-chemical state 112. Reversible transformation may be induced by applying the external stimuli in an amount sufficient to elicit mechanical motion of the capping agent 104 such that the active agents 108 are released from the pores 106, but such that the capping agent 104 does not rupture or disassociate from the external surface of the particle 102. In some embodiments, the capping agents 104 may be reversibly transformed from the first physical-chemical state 110 to the second physical-chemical state 112 in response to an increase of temperature in the region of interest (e.g., temperature of the bulk environment surrounding the capping agents 104). In some embodiments, the increase in temperature to effectuate the change from the first physical-chemical state 110 to the second physical-chemical state 112 may be 1° C., or 2° C., or 3° C., or 4° C., or 5° C., or 6° C., or 7° C., or 8° C., or 9° C., or 10° C., or more. The change in temperature may be relative to room temperature (e.g., about 20° C., or 21° C., or 22° C., or 23° C., or 24° C., or 25° C.).

In some embodiments, the capping agents 104 comprise a polymeric matrix. As used herein, the term “polymeric matrix” may refer to a composition (e.g., a matrix) comprising at least one polymer that may be coupled to the particle 102, and be configured to cover a sufficient portion of the pores 106 to prevent the release of active agents 108 received therein. The term “polymer” may refer to a macromolecule having repeating units connected by covalent bonds. In some embodiments, the capping agents 104 comprise hydrophilic polymers. Suitable polymers may comprise a polyether backbone, such as a polyalkylene glycol and, more particularly, polyethylene glycol (PEG). In some embodiments, the polyether backbone (e.g., PEG) may be transformed from the first physical-chemical state to the second physical-chemical state in response to a bulk temperature increase that ranges between 1° C. to 5° C. in the region surrounding the polyether backbone relative to room temperature.

In some embodiments, the capping agents 104 comprise polyethylene glycol. The polyethylene glycol may have a number average molar mass (Mₙ) that can range from 400 Da to 25,000 Da. In some embodiments, the number average molar mass is at least 400 Da, is at least 600 Da, is at least 800 Da, or is at least 1000 Da, or is at least 1200 Da, or is at least 1300 Da, or is at least 1400 Da, or is at least 1500 Da, or is at least 1600 Da, or is at least 1700 Da, or is at least 1800 Da, or is at least 1900 Da, or is at least 2000 Da. In some embodiments, the number average molar mass is less than 2200 Da, or less than 2300 Da, or less than 2400 Da, or less than 2500 Da, or less than 2600 Da, or less than 2700 Da, or less than 2800 Da, or less than 2900 Da, or less than 3000 Da. In some embodiments, the number average molar mass is less than 4000 Da, or less than 5000 Da, or less than 6000 Da, or less than 7000 Da, or less than 8000 Da, or less than 9000 Da, or less than 10,000 Da, or less than 11,000 Da, or less than 12,000 Da, or less than 13,000 Da, or less than 14,000 Da, or less than 15,000 Da, or less than 16,000 Da, or less than 17,000 Da, or less than 18,000 Da, or less than 19,000 Da, or less than 20,000 Da, or less than 21,000 Da, or less than 22,000 Da, or less than 23,000 Da, or less than 24,000 Da, or less than 25,000 Da. The number average molecular mass (Mₙ) may be defined as the arithmetic mean having a formula of:

$$Mn = \frac{Σ {N}_{i} {M}_{i}}{Σ {N}_{i}}$$

where Nᵢ is the number of polymer molecules and Mᵢ is the molecular weight. The number average molecular mass may be determined by methods known to the skilled artisan, such as gel permeation chromatography, viscometry (Mark-Houwink equation), colligative methods, end-group determination by nuclear magnetic resonance (NMR), among others.

In some embodiments, the weight fraction (w/w) of capping agents 104 in the stimuli-responsive composition range from 5% to 35%, based on the total weight of the composition. In some embodiments, the weight fraction of the capping agents 104 in the stimuli-responsive composition is at least 5%, or at least 6%, or at least 7%, 8%, is at least 9%, is at least 10%, is at least 11%, is at least 12%, is at least 13%, is at least 14%, is at least 15%, is at least 16%, is at least 17%, is at least 18%, is at least 19%, is at least 20%. In some embodiments, the weight fraction (w/w) of the capping agents 104 in the stimuli-responsive composition is less than 21%, or is less than 22%, or is less than 23%, or is less than 24%, or is less than 25%, or is less than 26%, or is less than 27%, or is less than 28%, or is less than 29%, or is less than 30%, or is less than 31%, is at least 32%, or is less than 33%, or is less than 34%, or is less than 35%. In some embodiments, the capping agents 104 comprise polyethylene glycol.

In some embodiments, the capping agents 104 are coupled to a surface on the particle 102 through a linking agent. The term “linking agent” as used herein may refer to a compound or moiety that couples the capping agents 104 to the surface of the particle 102. In some embodiments the linking agent covalently couples the capping agents 104 to the surface of the particle 102. In some embodiments, suitable linking agents may be from 2 to 30 carbon atoms in length, can include alkyl and heteroalkyl chains, cycloalkyls, heterocycloalkyls, aryls and heteroaryls, and combinations thereof. The types of bonds used to link the two components include, but are not limited to, carbon-carbon bonds, amides, amines, esters, carbamates, ureas, thioethers, thiocarbamates, thiocarbonate and thioureas. Exemplary linking agents include alkyl-amine compounds and amino-alkyl-silane compounds. Non-limiting examples include (3-Aminopropyl)triethoxysilane (APTES).

In some embodiments, the term “alkyl” as used herein may refer to a straight or branched, saturated, aliphatic radical having the number of carbon atoms indicated. For example, C₁-C₆ alkyl includes, but is not limited to, methyl, ethyl, propyl, isopropyl, butyl, isobutyl, sec-butyl, tert-butyl, pentyl, isopentyl, hexyl, etc. Other alkyl groups include, but are not limited to heptyl, octyl, nonyl, decyl, etc. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. The alkyl group is typically monovalent, but can be divalent, such as when the alkyl group links two moieties together.

Referring to FIG. 2, the stimuli-responsive compositions 100 may include capping agents 204 that comprise a macrocyclic molecule. As used herein, the term “macrocycle” may refer to a molecule or ion containing twelve or more membered ring. In some embodiments, the macrocyclic molecule includes glycouril monomers linked by methylene bridges. Exemplary macrocyclic molecules may include, but are not limited to, cucurbit[5]uril, cucurbit[6]uril, cucurbit[7]uril, cucurbit[8]uril, cucurbit[9]uril, and cucurbit[10]uril.

When in the first physical-chemical state 110, the macrocyclic molecule may be bound to the linking agent 220 such that the macromolecule covers a sufficient fraction of an opening to the pores 106 to prevent the release of the active agents 108 stored therein. The macromolecule may be bound to the linking agent 220 through non-covalent interactions, such as electrostatic interactions, van der Waals forces, hydrophobic effects, and π-effects.

In some embodiments, the macrocyclic molecules may have a diameter of at least 4 angstroms, at least 5 angstroms, at least 6 angstroms, at least 7 angstroms, at least 8 angstroms, at least 9 angstroms, at least 10 angstroms, at least 11 angstroms, at least 12 angstroms, at least 13 angstroms, at least 14 angstroms, at least 15 angstroms. In some embodiments, the macrocyclic molecules may have a diameter of less than 16 angstroms, less than 17 angstroms, less than 18 angstroms, less than 19 angstroms, or less than 20 angstroms

Referring back to FIG. 2, the capping agents 204 may be transformed from the first physical-chemical state 110 to the second physical-chemical state 112 in response to an external stimuli or trigger applied in an effective amount. For example, the capping agents 204 may be bound to the linking agent 220 through a binding constant that is temperature dependent. When the external stimulus 114, such as ultrasound or high-intensity focused ultrasound, is applied to the capping agents 204 in an effective amount the binding constant of the capping agents 204 and the linking agent 220 is decreased, and the capping agents 204 may be detached from the linking agent 220. Thus, the active agents 108 may then be selectively released from the pores 106 of the particles 102.

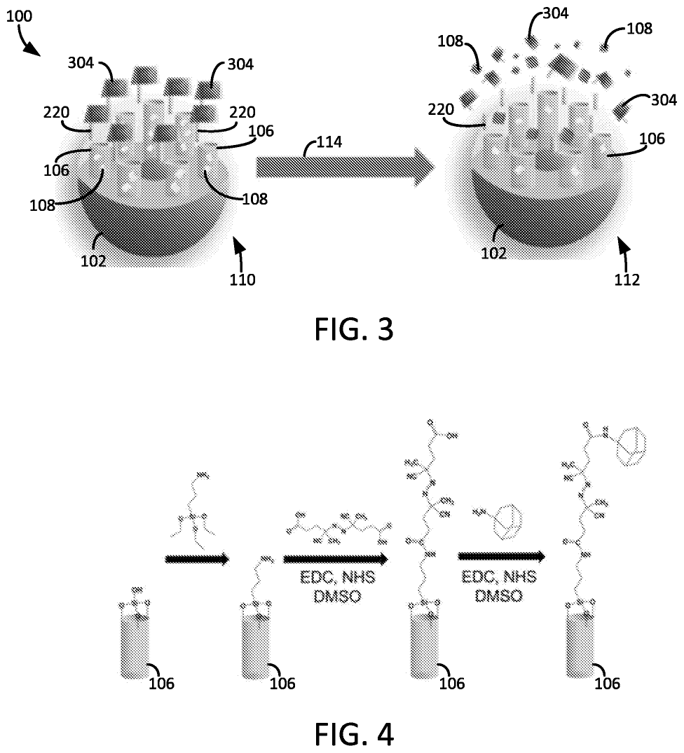

Referring to FIGS. 3-4, the stimuli-responsive compositions 100 may include capping agents 304 that comprise one or more ultrasound labile compound or species. In some embodiments, the ultrasound labile species or compounds may comprise one or more azo bond (N═N) along the length of the capping agent 304. As indicated by process block 114, the capping agents 304 may be transformed from the first physical-chemical state 110 to the second physical-chemical state 112 in response to an external stimuli applied to the ultrasound labile species or compound. When applied in an effective amount, the azo molecule may be cleaved and decompose into nitrogen. Thus, the capping agents 304 may be removed, and the active agents 108 stored within the pores 106 may be released.

In some embodiments, the capping agents 304 include an alkyl-azo compound or aliphatic azo compounds. In some embodiments, the capping agents 304 may further include a nano-valve covalently or non-covalently bound along the length of the aliphatic azo molecule. Suitable nano-valves include those having a diameter that covers a sufficient fraction of an opening to the pores 106 to prevent the release of active agents 108 stored therein. Exemplary nano-valves that could be bound to the aliphatic azo molecule include those formed from three or more cycloalkyl rings, such as adamantane (C₁₀H₁₆), and derivatives thereof. Additionally or alternatively, the nano-valves may include cyclodextrin complexes, and derivatives thereof. In some embodiments, the alkyl moiety in the alkyl-azo compound is sized such that the distance between the opening to the pores 106 and the nano-valves is sufficient to prevent the release of active agents 108 from within the pores. A linking agent 220 may bind the capping agent 304 to a surface of the particle 102. In some embodiments, the capping agent 304 includes 4,4′-azobis(4-cyanovaleric acid).

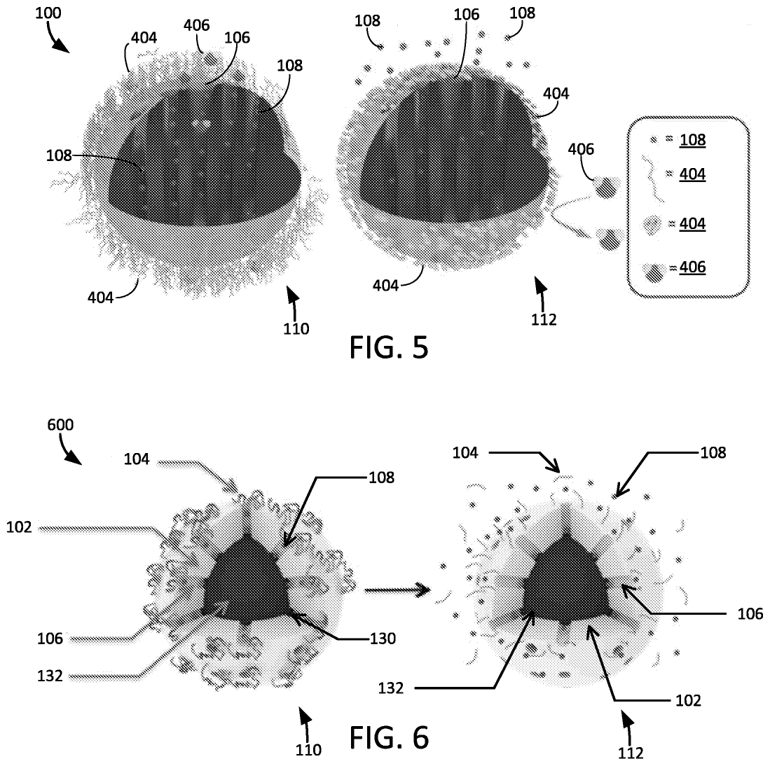

Referring to FIG. 5, the stimuli-responsive compositions 100 may include capping agents 404 that comprise a thermo-responsive polymer. In some embodiments, the thermo-responsive polymer comprises a reversible hydrophilicity. For example, the capping agents 404 may comprise one or more polymer that has a lower critical solution temperature (LCST) where the polymer is hydrophilic under the LCST and hydrophobic above the LCST, or vis versa. When in the first physical-chemical state 110, the thermo-responsive polymer may be configured to cover a sufficient fraction of an opening to the pores 106 to prevent the release of active agents 108 stored therein. The thermo-sensitive polymer may be transformed from the first physical-chemical state 110 to the second physical-chemical state 112 in response to an external stimuli applied to the compound or moiety in an effective amount.

Exemplary thermo-responsive polymers include, but are not limited to, poly(N-isopropylacrylamide) (PNIPAm). Poly(N-isopropylacrylamide) (PNIPAm) is a thermo-responsive polymer with a lower critical solution temperature (LCST) of 32° C. PNIPAm changes its hydrophilicity reversibly. For example, PNIPAm is hydrophilic under LCST and hydrophobic over LCST. Thus, PNIPAm may be transformed from a first physical-chemical state 110 (e.g., hydrophilic state) to a second physical-chemical state 112 (e.g., hydrophobic) in response to an external simtuli applied in an effective amount to increase PNIPAm above its LCST. If placed in an aqueous environment 406, the bulk size distribution of PNIPAm is controllable using an external stimuli, such as ultrasound. For example, the external stimuli may be applied to exceed the LCST and, in response, the size of capping agents 404 will decrease thereby allowing active agents 108 to be released from the pores 106. In some embodiments, PNIPAm may decrease in size by 90% when heated above the LCST due to the repulsion from surrounding aqueous environment (e.g., water).

In some embodiments, the stimuli-responsive compositions 100 include capping agents that comprise a light-sensitive compound or species. The light-sensitive compound or species may be coupled to the particle 102 through a linking agent, as described above. When in the first physical-chemical state 110, the light sensitive compound or moiety may be bound to the linking agent such that the compound or moiety covers a sufficient fraction of an opening to the pores 106 to prevent the release of active agents 108 stored therein. The light-sensitive may be transformed from the first physical-chemical state 110 to the second physical-chemical state 112 in response to an external stimuli applied to the compound or moiety in an effective amount.

In some embodiments, the light-sensitive compound is light-labile. Exemplary light-labile compounds include, but are not limited to, cyclodextrins. The cyclodextrins may be bound to the linking agent to block the pore 106 entrance. On light excitation the cyclodextrin may move a sufficient distance to allow the passage of the active agents 108 from the pore 106. In some embodiments, the cyclodextrin may dissociate from the linking agent. In some embodiments, a bulky molecule, such as adamantine, may be coupled to the end of the linking agent to prevent dissociation of the cyclodextrin compound.

In some embodiments, the geometric orientation or geometric isomerism of the light-sensitive compound may be altered in response to light stimulation. For example, the light-sensitive compound may be reversibly altered between isomeric states, such as cis and trans orientations, via photoisomerization. Exemplary light-sensitive compounds may include, but are not limited to, azoaryl compounds, such as azobenzene and derivatives thereof. Prior to light stimulation, azobenzene molecules in the trans isomer are hydrophobic, immobile, and hinder the release of the active agents from the pores 106 of the particles 102. On light stimulation, the azobenzenes switch to a cis configuration

Referring to FIG. 6, a stimuli-responsive composition 600 is illustrated according to some embodiments of the present disclosure. The stimuli-responsive composition 600 includes a particle 102 that forms a shell having a hollow chamber 130 disposed therein. In some embodiments, the hollow chamber 130 is in fluid communication with the pores 106 such that active agents 108 may enter and exit the hollow chamber 130 through the pores 106. The hollow chamber 130 may be configured to include one or more additional particle 132 disposed therein. In some embodiments, the one or more additional particle 132 comprises a paramagnetic particle and, more particularly, a superparamagnetic particle. The particle 102 may include one or more capping agent (e.g., 104-504) coupled to a surface of the particle 102 as described in any of the preceding embodiments.

Exemplary additional particles 132 include, but are not limited to, iron oxide particles. In some embodiments, the iron oxide particle may include a dopant, such as manganese (Mn), zinc (Zn), cobalt (Co), among others. Non-limiting example additional particles 132 include MnFe₂O₄, Zn_{0.4}Fe_{2.6}O₄, or CoFe₂O₄.

In some embodiments, the particle 102 is directly coupled to the outer surface of the additional particle 132. Alternatively, the additional particle 132 may have a dimension (e.g.,) that is less than the internal diameter of the hollow chamber 130.

In some embodiments, the particle 102 may have a shell thickness of 5 nm to 100 nm. In some embodiments, the particle 102 may have a shell thickness of at least 5 nm, at least 10 nm, at least 15 nm, at least 20 nm, at least 25 nm, at least 30 nm, at least 35 nm, at least 40 nm, at least 45 nm, or to at least 50 nm. In some embodiments, the particle 102 has a shell thickness less than 55 nm, less than 60 nm, less than 65 nm, less than 70 nm, less than 75 nm, less than 80 nm, less than 85 nm, less than 90 nm, less than 95, or less than 100 nm, or less than 200 nm, or less than 300 nm, or less than 400 nm, or less than 500 nm, or less than 1000 nm, or less than 2000 nm.

In some embodiments, one or more additional particle 132 may have a dimension on the nanometer scale that is less than the internal diameter of the particle 102. In some embodiments, the particle 102 contains at least one additional particle 132, or at least two, or at least three, or at least four, or at least five, or less than 6, or less than 7, or less than 8, or less than 9, or less than 10, or less than 50, or less than 100 additional particles 132.

In some embodiments, the dimension of the one or more additional particle 132 ranges from 1 nm to 50 nm. In some embodiments, the dimension of the one or more additional particle 132 is at least 0.1 nm, or at least 1 nm, or at least 5 nm, or at least 10 nm, or at least 15 nm, or at least 20 nm. In some embodiments, the one or more additional particle 132 has a dimension of less than 25 nm, or less than 30 nm, or less than 35 nm, or less than 40 nm, or less than 45 nm, or less than 50 nm.

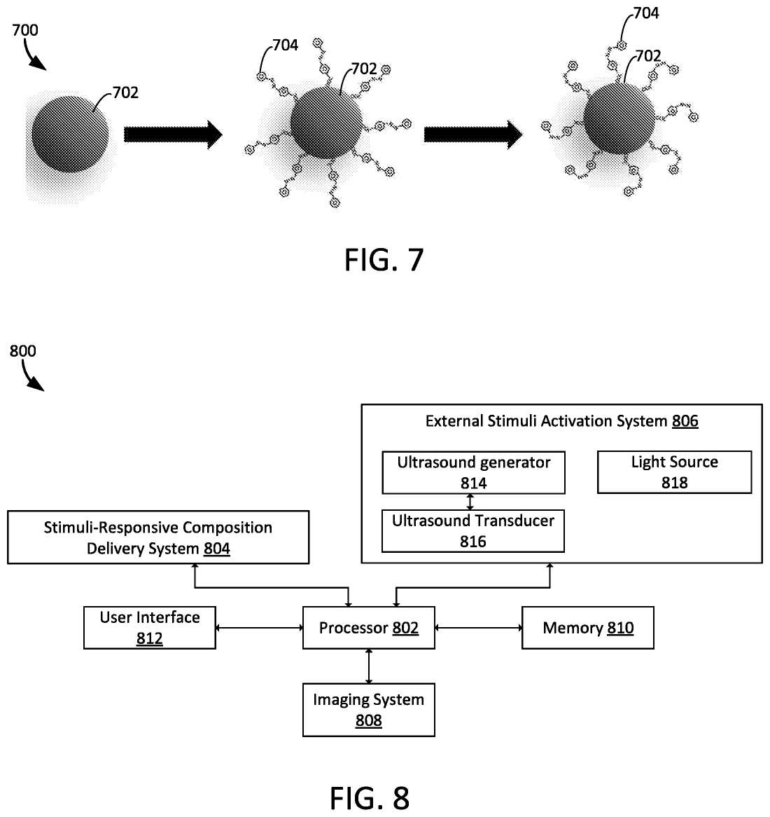

Referring to FIG. 7, a stimuli-responsive composition 700 is illustrated in accordance with one embodiment of the present disclosure. The stimuli-responsive composition 700 includes one or more particle 702 formed from one or more biocompatible material, such biocompatible metal complexes, biocompatible metal oxides, biocompatible mixed metal oxides, biocompatible semi-metal oxides, biocompatible mixed semi-metal oxides, and combinations thereof. Suitable biocompatible materials include those that may transform from a first physical state 110 to a second physical state 112 in response to an external stimuli, such as ultrasound or light.

In some embodiments, the particles 702 include materials that undergo spin-crossover from a diamagnetic state, S=0 (e.g., first physical-chemical state 110), to a paramagnetic state, S=2 (second physical-chemical state 112), in response to the application of an external stimuli applied in an effective amount. Exemplary particles 702 include, but are not limited to, iron complexes and iron particles. Suitable iron particles include iron oxide particles and, more particularly, ultrasmall iron oxide nanoparticles (USIONs). In some embodiments, the iron oxide particles include a dimension on the nanometer scale that can range from 1 to 10 nm.

In some embodiments, the iron oxide particles have a diameter of at least 1 nm, of at least 2 nm, or at least 3 nm, of at least 4 nm, of at least 5 nm, of at least 6 nm, of at least 7 nm, of at least 8 nm, of at least 9 nm, or of at least 10 nm.

Alternatively or additionally, the particles 702 may comprise iron(II) complexes. Non-limiting examples include six coordinate iron(II) complexes, such as Fe(Me-bik)₃](BF₄)₂.25H₂O. Fe(Me-bik)₃](BF₄)₂.25H₂O comprises a an octahedral structure with an octahedral {N6} coordination polyhedron. At low temperature the compound is in its diamagnetic low-spin (LS) configuration (t₂ g⁶, S=0) and is converted into a paramagnetic high-spin (HS) electron configuration (t₂ g⁴ e_{g}², S=2) at higher temperatures. An external stimuli, such as ultrasound or light, may be used to transform the iron(II) complex from the first physical-chemical state 110 to the second physical-chemical state 112.

In some embodiments, the particles 702 include capping agents 704 coupled to a surface of the particle 702. Exemplary capping agents 704 for the particles 702 include light-sensitive capping agents. For example, the light-sensitive compound may be reversibly altered between isomeric states, such as cis- and trans-orientations, via photoisomerization. Exemplary light-sensitive compounds may include, but are not limited to, azoaryl compounds, such as carboxyazobenzene, aminoazobenzene, azobenzene, and derivatives thereof.

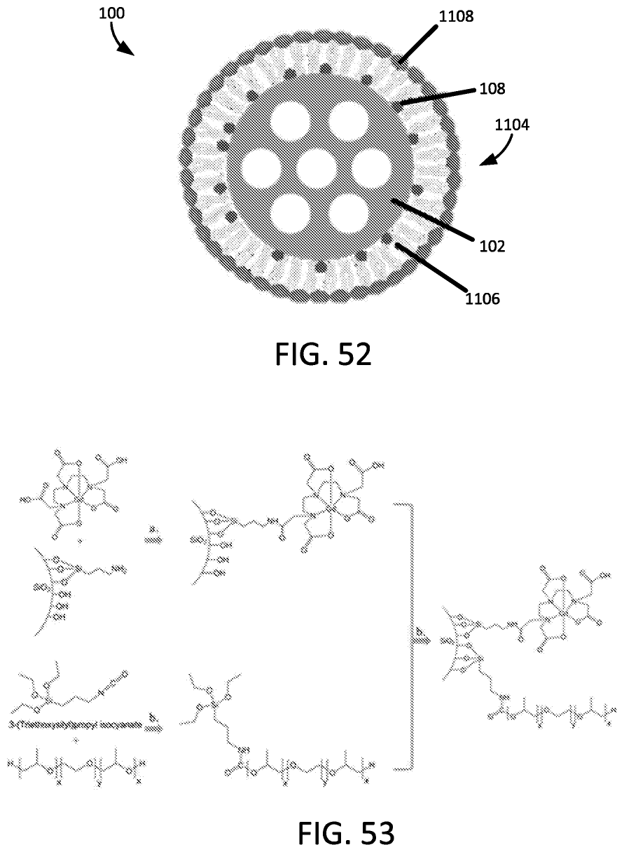

Referring to FIGS. 52-53, in some embodiments, the stimuli-responsive composition 100 includes one or more capping agent 1104 coupled to a surface of the particle 102. In some embodiments, the capping agent 1104 comprises a mechano-responsive polymer that is configured to vibrate or move in response to an external stimulus. In some embodiments, the stimuli-responsive composition 100 includes an active agent 108 coupled to the surface of the particle 102 (e.g., through a covalent bond). In some embodiments, a fraction of the active agents 108 may be coupled to the surface of the particle 102 and another fraction may be configured in the pores of the particle. In some embodiments, all the active agents 108 are coupled to the surface of the particle 102.

In some embodiments, the mechano-responsive capping agent 1104 comprises a poloxamer. Suitable poloxamers include multiblock polymers comprising hydrophobic regions 1108 and hydrophilic regions 1106. In some embodiments, the capping agent 1104 is a poloxamer comprising poly(propylene oxide) (PO) and poly(ethylene oxide) (EO). In some embodiments, the capping agent 1104 is a nonionic triblock polymer composed of a central hydrophobic chain flanked by two hydrophilic chains, such as poly(ethylene oxide)/poly(propylene oxide)/poly(ethylene oxide) (i.e., EO/PO/EO). Alternatively, in some embodiments, the hydrophilic chain is in the middle and hydrophobic chain at two ends, such as poly(propylene oxide)/poly(ethylene oxide)/poly(propylene oxide) (i.e., PO/EO/PO).

In some embodiments, the active agent 108 and the poloxamer capping agent 1104 are covalently bonded to the surface of the particle 102. When external stimulus (e.g., HIFU) is not applied, the active agent is surrounded by a hydrophobic region of the poloxamer, and therefore solvent access to the active agent is reduced. In the instance where the active agent 108 is a contrast agent (e.g., Gd-DTPA), there is less solvent access to the contrast agent resulting in longer T₁ relaxation times. During external stimulus, vibration or motion of the poloxamer is induced, which generates cavities. At this time, the solvent permeability of the poloxamer is increased, and the solvent can contact the active agent 108. In the instance where the active agent 108 is a contrast agent, this results in shorter T₁ relaxation time. If the poloxamer is covalently bonded to the particle 102, the poloxamer structure will recover when the external stimulus is turned off, and the solvent will be pushed away from the active agent 108, resulting in the T₁ relaxation time returning to its initial value. In this way, periodic external stimulus (e.g., HIFU) may generate reversible T₁ contrast change.

Referring to FIG. 53, the active agent 108 may be covalently bonded to the surface of the particle 102, for example, though an EDC/NHS reaction. The poloxamer may be covalently bonded to the surface of the particle 102, for example, by using (triethoxysilyl)propyl isocyanate and then condensing (e.g., refluxing in toluene) the poloxamer to the surface of the particle 102.

In some embodiments, the capping agent 1104 has a molecular weight (Mn) from 2000 to 6500. In some embodiments, the molecular weight of the capping agent 1104 is greater than 2000, greater than 2200, greater than 2400, greater than 2600, greater than 2800, greater than 3000, greater than 3200, greater than 3400, greater than 3600, greater than 3800, or greater than 4000. In some embodiments, the molecular weight is less than 4200, or less than 4400, or less than 4600, or less than 4800, or less than 5000, or less than 5200, or less than 5400, or less than 5600, or less than 5800, or less than 6000, or less than 6200, or less than 6500.

In some embodiments, the EO content in the poloxamer is from 5% (w/w) to 45% (w/w), based on the total weight of the poloxamer. In some embodiments, the EO content is greater than 5% (w/w), or greater than 10%, or greater than 15%, or greater than 20%, or greater than 25% (w/w), based on total weight of the poloxamer. In some embodiments, the EO content is less than 30% (w/w), or less than 35%, or less than 45% (w/w), based on the total weight of the poloxamer. In some embodiments, the PO content balances out the composition to 100% (w/w) and may range from 55% to 95% (w/w) based on total weight of the poloxamer.

In some embodiments, the capping agent 1104 and the particle 102 have a hydrodynamic diameter from 100 nm to 400 nm. In some embodiments, the hydrodynamic diameter is greater than 100 nm, or greater than 150 nm, or greater than 200 nm. In some embodiments, the hydrodynamic diameter is less than 250 nm, or less than 300 nm, or less than 400 nm.

Referring particularly now to FIG. 8, an example of an apparatus 800 that can implement the methods of the present disclosure is illustrated. In general, the apparatus 800 includes a processor 802 that is configured to be in electrical communication with a variety of components. In some embodiments, the processor 802 may communicate with an external stimuli activation system 806. Additionally, the processor 802 may optionally communicate with a stimuli-responsive composition delivery system 804 and an imaging system 808. The stimuli-responsive composition delivery system 804 may be configured to administer one or more stimuli-responsive composition to a target region of a subject. The imaging system 808 may be configured to acquire one or more image of the target region of the subject.

The processor 802 includes a commercially available programmable machine running on a commercially available operating system. That is, the processor 802 is configured with a memory 810 having stored programmable instructions therein. The processor 802 may be capable of communicating with the external stimuli activation system 806, the optional stimuli-responsive composition delivery system 804, and the optional imaging system 808 to process data based on programmable instructions stored in the memory 810, and generate instructions therefrom.

The processor 802 may be coupled to a user interface 812 that allows input parameters (e.g., operational parameters) to be entered into the external stimuli activation system 806, the optional imaging system 808, and the optional stimuli-responsive composition delivery system 804. The user interface 812 may be a switch or button or collection of switches or buttons. Also, the user interface 812 may include other interface components, such as displays or touch screens. To that end, the user interface 812 may also display the results. The processor 802 may communicate with each of these systems, or components thereof, through any suitable network connection, whether wired, wireless, or a combination of both.

In some embodiments, the external stimuli activation system 806 functions in response to the processor 802 to apply external stimuli to a target region of a subject. The external stimuli activation system 806 may optionally include a commercially available ultrasound system having an ultrasound generator 814 and transducer 816 that are configured to apply ultrasound to the target region of the subject. In some non-limiting examples, the ultrasound system comprises a commercially available high-intensity focused ultrasound (HIFU) system.

Additionally or alternatively, the external stimuli activation system 806 may include a light source 818 configured to apply light (e.g., electromagnetic radiation) to the target region on the subject. Suitable light sources 816 include commercially available lasers, lamps, light emitting diodes, or other sources of electromagnetic radiation. Light radiation can be supplied in the form of a monochromatic laser beam, e.g., an argon laser beam or diode-pumped solid state laser beam. Light can also be supplied to a non-external surface tissue through an optical fiber device, e.g., the light can be delivered by optical fibers threaded through a small gauge hypodermic needle or an arthroscope. Light can also be transmitted by percutaneous instrumentation using optical fibers or cannulated waveguides.

In some embodiments, the external stimuli activation system 806 may apply ultrasound to the target region of the subject at an output power from 1 W to 1000 W. In some embodiments, the output power is greater than 1 W, or greater than 5 W, or greater than 10 W, or greater than 20 W, or greater than 30 W, or greater than 40 W, or greater than 50 W, or greater than 100 W, or greater than 200 W, or greater than 250 W, or greater than 300 W. In some embodiments, the power output is less than 400 W, or less than 500 W, or less than 600 W, or less than 800 W, or less than 1000 W.

In some embodiments, the external stimuli activation system 806 may apply the ultrasound to the target region for a duration. In some embodiments, the duration is from 0.01 seconds to 30 minutes. In some embodiments, the duration is greater than 0.01 s, or greater than 0.1 s, or greater than 1 s, or greater than 5 s, or greater than 10 s, or greater than 20 s, or greater than 30 s, or greater than 1 min, or greater than 5 min, or greater than 10 min. In some embodiments, the duration is less than 15 min, or less than 20 min, or less than 25 min, or less than 30 min. In some embodiments, the ultrasound is applied periodically.

In some embodiments, the external stimuli activation system 806 may apply ultrasound to the target region at a frequency. In some embodiments, the frequency ranges from 100 mHz to 20 kHz. In some embodiments, the frequency is greater than 20 kHz, or greater than 30 kHz, or greater than 40 kHz, or greater than 50 kHz, or greater than 60 kHz, or greater than 70 kHz, or greater than 100 kHz, or greater than 500 kHz, or greater than 1 MHz, or greater than 5 MHz, or greater than 10 MHz, or greater than 15 MHz, or greater than 20 MHz. In some embodiments, the frequency is less than 25 MHz, or less than 30 MHz, or less than 40 MHz, or less than 50 MHz, or less than 60 MHz, or less than 70 MHz, or less than 80 MHz, or less than 90 MHz, or less than 100 MHz.

In some embodiments, the optional stimuli-response composition delivery system 804 is configured to administer the one or more stimuli-responsive composition to a target region of a subject. As used herein, the term “administer,” “administering,” or “administration” may refer to the delivering the stimuli-responsive composition to a subject. Administration may be by any appropriate route. For example, in some embodiments, administration may be interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, bronchial (including by bronchial instillation), buccal, enteral, vaginal and vitreal. The external stimuli-response composition delivery system 804 may include any commercially available equipment to accomplish the aforementioned administration techniques. In some embodiments, the optional stimuli-responsive composition delivery system 804 functions in response to the processor 802 to administer the stimuli-responsive composition to the subject (e.g., operate a pump to transfer the composition from a vessel to the subject).

In some embodiments, the imaging system 808 functions in response to instructions received from the processor 802 to acquire an image of the target region during and/or after application of the external stimuli. Suitable imaging systems 808 include any commercial system configured to create visual representations of the interior of a body for clinical analysis and medical intervention, as well as visual representation of the function of some organs or tissues. Exemplary systems include medical imaging devices, such as but not limited to: X-ray radiography, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography (SPECT).

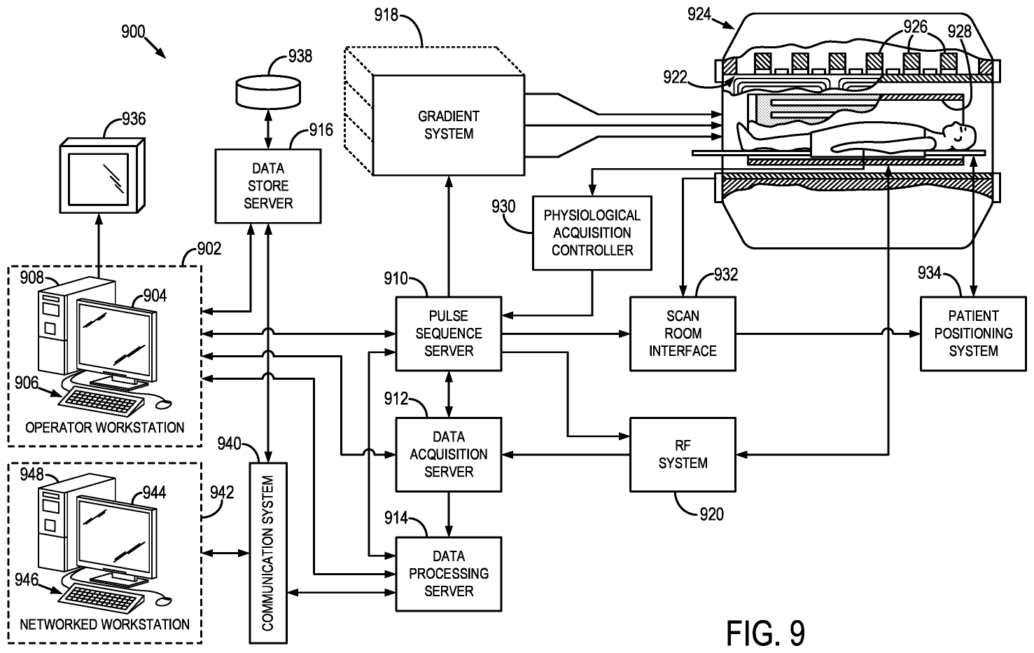

Referring to FIG. 9, an MRI system 900 is illustrated that that can implement the MRI acquisition methods described herein. The MRI system 900 includes an operator workstation 902 that may include a display 904, one or more input devices 906 (e.g., a keyboard, a mouse), and a processor 908. The processor 908 may include a commercially available programmable machine running a commercially available operating system. The operator workstation 902 provides an operator interface that facilitates entering scan parameters into the MRI system 900. The operator workstation 902 may be coupled to different servers, including, for example, a pulse sequence server 910, a data acquisition server 912, a data processing server 914, and a data storage server 916. The operator workstation 902 and the servers 910, 912, 914, and 916 may be connected via a communication system 940, which may include wired or wireless network connections.

The pulse sequence server 910 functions in response to instructions provided by the operator workstation 902 to operate a gradient system 918 and a radiofrequency (“RF”) system 920. Gradient waveforms for performing a prescribed scan are produced and applied to the gradient system 918, which then excites gradient coils in an assembly 922 to produce the magnetic field gradients Gₓ, G_{y}, and G_{z} that are used for spatially encoding magnetic resonance signals. The gradient coil assembly 922 forms part of a magnet assembly 924 that includes a polarizing magnet 926 and a whole-body RF coil 928.

RF waveforms are applied by the RF system 920 to the RF coil 928, or a separate local coil to perform the prescribed magnetic resonance pulse sequence. Responsive magnetic resonance signals detected by the RF coil 928, or a separate local coil, are received by the RF system 920. The responsive magnetic resonance signals may be amplified, demodulated, filtered, and digitized under direction of commands produced by the pulse sequence server 910. The RF system 920 includes an RF transmitter for producing a wide variety of RF pulses used in MRI pulse sequences. The RF transmitter is responsive to the prescribed scan and direction from the pulse sequence server 910 to produce RF pulses of the desired frequency, phase, and pulse amplitude waveform. The generated RF pulses may be applied to the whole-body RF coil 928 or to one or more local coils or coil arrays.

The RF system 920 also includes one or more RF receiver channels. An RF receiver channel includes an RF preamplifier that amplifies the magnetic resonance signal received by the coil 928 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

M=\sqrt{I²+Q²}

and the phase of the received magnetic resonance signal may also be determined according to the following relationship:

$$φ = {tan}^{- 1} ( \frac{Q}{I} )$$

The pulse sequence server 910 may receive patient data from a physiological acquisition controller 930. By way of example, the physiological acquisition controller 930 may receive signals from a number of different sensors connected to the patient, including electrocardiograph (“ECG”) signals from electrodes, or respiratory signals from a respiratory bellows or other respiratory monitoring devices. These signals may be used by the pulse sequence server 910 to synchronize, or “gate,” the performance of the scan with the subject's heart beat or respiration.

The pulse sequence server 910 may also connect to a scan room interface circuit 932 that receives signals from various sensors associated with the condition of the patient and the magnet system. Through the scan room interface circuit 932, a patient positioning system 934 can receive commands to move the patient to desired positions during the scan.

The digitized magnetic resonance signal samples produced by the RF system 920 are received by the data acquisition server 912. The data acquisition server 912 operates in response to instructions downloaded from the operator workstation 902 to receive the real-time magnetic resonance data and provide buffer storage, so that data is not lost by data overrun. In some scans, the data acquisition server 912 passes the acquired magnetic resonance data to the data processor server 914. In scans that require information derived from acquired magnetic resonance data to control the further performance of the scan, the data acquisition server 912 may be programmed to produce such information and convey it to the pulse sequence server 910. For example, during pre-scans, magnetic resonance data may be acquired and used to calibrate the pulse sequence performed by the pulse sequence server 910. As another example, navigator signals may be acquired and used to adjust the operating parameters of the RF system 920 or the gradient system 918, or to control the view order in which k-space is sampled. In still another example, the data acquisition server 912 may also process magnetic resonance signals used to detect the arrival of a contrast agent in a magnetic resonance angiography (“MRA”) scan. For example, the data acquisition server 912 may acquire magnetic resonance data and processes it in real-time to produce information that is used to control the scan.

The data processing server 914 receives magnetic resonance data from the data acquisition server 912 and processes the magnetic resonance data in accordance with instructions provided by the operator workstation 902. Such processing may include, for example, reconstructing two-dimensional or three-dimensional images by performing a Fourier transformation to raw k-space data, performing other image reconstruction algorithms (e.g., iterative or back-projection reconstruction algorithms), applying filters to raw k-space data or to reconstructed images, generating functional magnetic resonance images, or calculating motion or flow images.

Images reconstructed by the data processing server 914 are conveyed back to the operator workstation 902 for storage. Real-time images may be stored in a data base memory cache, from which they may be output to operator display 902 or a display 936. Batch mode images or selected real time images may be stored in a host database on disc storage 938. When such images have been reconstructed and transferred to storage, the data processing server 914 may notify the data store server 916 on the operator workstation 902. The operator workstation 902 may be used by an operator to archive the images, produce films, or send the images via a network to other facilities.

The MRI system 900 may also include one or more networked workstations 942. For example, a networked workstation 942 may include a display 944, one or more input devices 946 (e.g., a keyboard, a mouse), and a processor 948. The networked workstation 942 may be located within the same facility as the operator workstation 902, or in a different facility, such as a different healthcare institution or clinic.

The networked workstation 942 may gain remote access to the data processing server 914 or data store server 916 via the communication system 940. Accordingly, multiple networked workstations 942 may have access to the data processing server 914 and the data storage server 916. In this manner, magnetic resonance data, reconstructed images, or other data may be exchanged between the data processing server 914 or the data storage server 916 and the networked workstations 942, such that the data or images may be remotely processed by a networked workstation 942.

In some embodiments, an MRI acquisition and post-processing method is provided that takes advantage of periodic modulation of the stimuli-responsive compositions by external stimuli, such as ultrasound (e.g., HIFU) or light, to generate on-command contrast enhancement from the stimuli-responsive compositions while suppressing background signal.

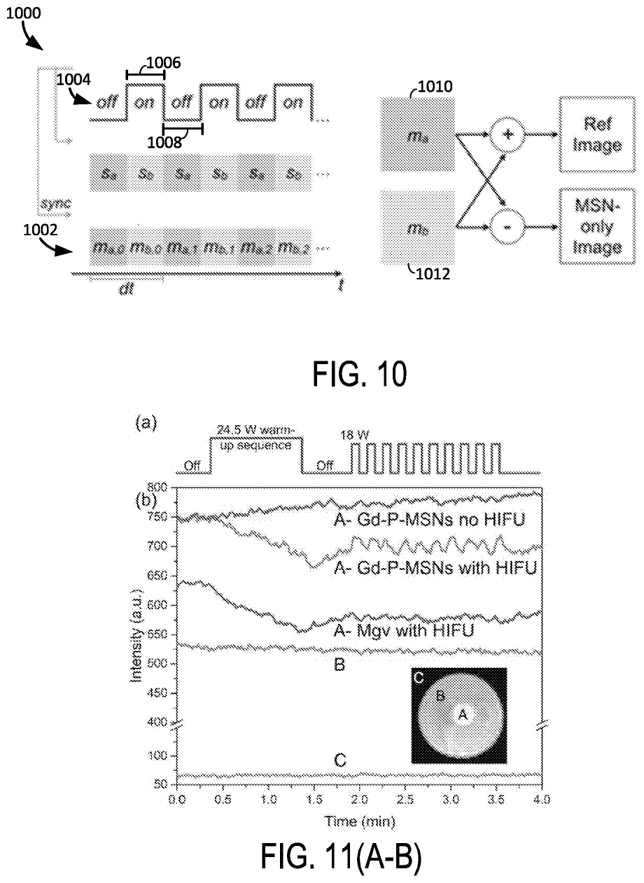

Referring now to FIG. 10, a flowchart is illustrated as setting forth the steps of an example MRI acquisition technique 1000 in accordance with the present disclosure. In some embodiments, the method includes acquiring data through a synchronized MRI acquisition block 1002 that is implemented as a stimulus-triggered pulse sequence. That is, an external stimulus may be applied to the target region of the subject in an acquisition block 1004, where the external stimulus is be applied for a first duration 1006 and turned off for a second duration 1008.

In some embodiments, a stimulus modulation time scale (dt) may be defined as the summed total of the first duration 1006 and the second duration 1008 (e.g., total time between respective on-off cycles of external stimulus). In some embodiments, the external stimulus is applied to the target region during the first duration for a time or dosage sufficient to transform the stimuli-responsive composition from the first state Sₐ to the second state S_{b}. In some embodiments, the first duration 1006 and the second duration 1008 may be controlled to be the same throughout the acquisition block 1004. Alternatively or additionally, the first duration 1006 and the second duration 1008 may be varied in a regular manner, random manner, or a pseudo-random manner throughout the acquisition block 1004. In some embodiments, the stimulus modulation time scale may be on the order of milliseconds (ms) to minutes. The stimulus modulation time may be adapted based on the stimuli-responsive compositions.

In some embodiments, MRI T₁ data are acquired in each acquisition block m_{a,i} or m_{b,i} (i=0 . . . N) using a rapid T₁-weighted sequence. Exemplary T₁-weighted sequences include, but are not limited to, inversion-recovery pulses (IR), saturation-recovery (SR) prepared turbo spin echo (TSE), steady-state free precession (SSFP), or variable flip-angle gradient echo (GRE) pulses. In some embodiments, MRI T₂ data are acquired in each acquisition block m_{a,i} or m_{b,i} (i=0 . . . N) using T₂-weighted sequences, such as TSE, T₂-prepared GRE, or SSFP. T₂* data can be acquired by T₂*-weighted GRE with different echo time (TE) values.

In some aspects, echo-planar imaging (EPI) or fast sampling trajectories (e.g., radial, spiral) may be used to accelerate MRI acquisition speed for fast stimulus modulation time scales (e.g., dt=100 ms to 1 sec). Additionally or alternatively, undersampled subsets of MRI data may be acquired for each m_{a,i} or m_{b,i} (i=0 . . . N) (e.g., dt=10-100 ms), and a full MR image may be completely acquired over one or more cycles of external stimulation.

In some embodiments, rapid dynamic sequences, such as golden-angle-ordered 2D radial or 3D stack-of-radial, may be used to continually acquire data (e.g., free-running acquisition without synchronization or triggering) throughout multiple cycles of periodic modulation by external stimuli. The sequence imaging parameters can be specified to have sensitivity to one or more MRI contrast parameters (e.g., T₁ or T₂). The temporal frame rate of the MRI acquisition can be made high enough to capture multiple image frames within each modulation period (e.g., 10 frames during dt=30 sec).

In some embodiments, repeated synchronized acquisitions of m_{a,i} or m_{b,i} (i=0 . . . N) with the same parameters over one or multiple stimulation cycles (e.g., N=4, 5, 6, 7, 8, 9, 10, etc.) may be used to enhance detection sensitivity and specificity. In addition, several m_{a,i} or m_{b,i} (i=0 . . . N) with different contrast weighting parameters (e.g., inversion recovery or saturation recovery timing for T₁ weighting, TE for T₂ or T₂* weighting) can be obtained to calculate quantitative relaxation time maps (e.g., T₁ or T₂) for each state of the stimuli-responsive composition (e.g., first physical-chemical state 110 and second physical-chemical state 112). The difference between the acquired MR images mₐ and m_{b} (or relaxation time maps) at different states creates images (or ΔT₁ maps) with suppressed background and signal specific to the stimuli-responsive compositions. At a minimum, one MR image without stimulation 1010 and one MR image during stimulation 1012 should be acquired and the difference can be used to capture the contrast enhancement.

In some embodiments, both the synchronized acquisition data and free-running acquisition data can be analyzed using spectral analysis of the signal changes over time (e.g., Fourier transform), or by correlating the time domain signal to a known modulation function (e.g., sinusoidal function with the applied modulation period). This approach may have improved results when multiple MR images are acquired for each period of simulation (increases spectral bandwidth) and throughout multiple cycles of stimulation (increases spectral resolution). One example is to create a spatial map of the integrated area under a specific spectral peak (i.e., frequency). The spatial map can be overlaid on a reference MR image to highlight areas with stimuli-modulated contrast enhancement, as illustrated in FIG. 11. The temporal resolution (or update rate of the contrast enhancement images) can be improved by using sliding-window reconstruction and spectrogram analysis.

In some embodiments, several parameters can be adjusted to improve the contrast enhancement performance (e.g., to optimize contrast-to-noise ratio per unit acquisition time), for example, based on the stimuli-responsive composition, type of external stimulation, energy level of the external stimulation (e.g., HIFU power), period and cycles of the modulated stimulation, type of MRI sequence, MRI sequence parameters, and image reconstruction/processing method.

When more than one MRI parameter (e.g., T₁, T₂, T₂*) is changing during the periodic modulation of the stimuli-responsive composition by external stimulation, the changes may be combined by image processing (e.g., linear combinations, multiplication, exponentiation) to further enhance the contrast. The on-command contrast enhancement generated herein significantly suppresses background signal and achieves specific highlighting of the stimuli-responsive composition at tissues of interest. The systems and methods described herein allow for repeated probing of the stimuli-responsive compositions, and tissues of interest for diagnosis, and/or therapeutic monitoring (as opposed to other existing systems where interrogation of the system for contrast enhancement irreversibly disrupts the system). For certain types of stimuli-responsive compositions, techniques provided herein further enables monitoring of titrated release and dose control of encapsulated agents.

### EXAMPLES

The following examples set forth, in detail, ways in which the stimuli-response compositions 100 may be synthesized, methods of using the stimuli-responsive compositions 100, and methods of acquiring image data using the imaging systems (e.g., 800). The following examples will enable one of skill in the art to more readily understand the principles thereof. The following examples are presented by way of illustration and are not meant to be limiting in any way.

### Example 1: Exemplary Materials, Methods of Fabrication, and Characterization of Silica Particles Configured with Capping Molecules Having a Polyether Backbone as Described Herein

### Synthesis of Silica Particles:

Mesoporous silica nanoparticles (MSNs) were synthesized using a sol-gel reaction in the presence of cationic surfactant templates. Hexadecyltrimethylammonium bromide CTAB (250 mg) was dissolved in a mixture of 120 mL of deionized H₂O and 875 μL of NaOH solution (2 M) in a 250 mL of round bottom flask with vigorous stirring. The solution was heated to 80° C. in an oil bath and kept at this temperature for 30 min followed by the dropwise addition of 1.25 mL of tetraethyl orthosilicate (TEOS). The reaction was stirred for another 2 h for the formation of MSNs. Afterwards, the solution was cooled to room temperature, centrifuged, and washed with ethanol three times to remove unreacted precursors, and free surfactants.

### Synthesis of Linking Agent Functionalized Silica Particles:

To functionalize a linking agent to the surface of the silica particle, an amine group was coupled to the surface of MSNs. For example, MSNs (180 mg) were dispersed in the mixture 50 mL of anhydrous toluene and 150 μL of APTS. The reaction was heated to 110° C. and refluxed for 12 h with vigorous stirring. After the reaction, APTS modified MSNs (MSNs-APTS) were washed with ethanol for 2 times. To remove the surfactant template, MSNs-APT S were dispersed in the mixture of 100 mL of ethanol and 2 g of ammonium nitrate (NH₄NO₃) in a 250 mL round bottom flask. The reaction was heated to 78° C. and refluxed for 1 h. The surfactant removal process by extraction was repeated twice. After the extraction, MSNs-APTS were centrifuged and further washed with deionized H₂O and ethanol twice, respectively. Finally, MSNs-APTS were stored in ethanol for further usage.

Loading Silica Particles with an Active Agent and PEG Capping:

Before loading Gd(DTPA)²⁻ in the mesopores of MSNs-APTS, MSNs-APTS were first washed with deionized H₂O. The loading of Gd(DTPA)²⁻ was carried out by soaking MSNs-APTS (20 mg) in 1 mL of gadopentetate dimeglumine solution (500 mM Gd(DTPA)²⁻) The solution was stirred overnight to let Gd(DTPA)²⁻ diffuse into the pores of MSNs-APTS. The Gd(DTPA)²⁻-loaded MSNs-APTS were then conjugated with HOOC-PEG-COOH (Mw=2000 Da) to seal the pores. EDC-HCl (15.3 mg) and sulfo-NHS (8.7 mg) coupling agents were dissolved in 240 μL of MES solution (100 mM, pH=6.0). So that both carboxylic acids on COOH-PEG-COOH could be activated, the PEG polymer (20 mg) dissolved in 200 μL of MES buffer solution was stepwise added into the solution of coupling agents every 10 min. Afterward, the activated PEG was stepwise added into the Gd(DTPA)²⁻-loaded MSNs-APTS solution every 10 min to enable both ends of the activated PEG to react with the primary amines on the surface of MSNs-APTS. The mixture was reacted overnight and then washed thoroughly with deionized H₂O to remove the unloaded Gd(DTPA)²⁻, the excess EDC-HCl and sulfo-NHS, and PEG. The resulting PEG-capped nanoparticles are denoted as MSNs-PEG. Finally, Gd(DTPA)²⁻-loaded MSNs-PEG were stored in 10 mL of deionized H₂O for further ultrasound-stimulated release studies.

### Characterization:

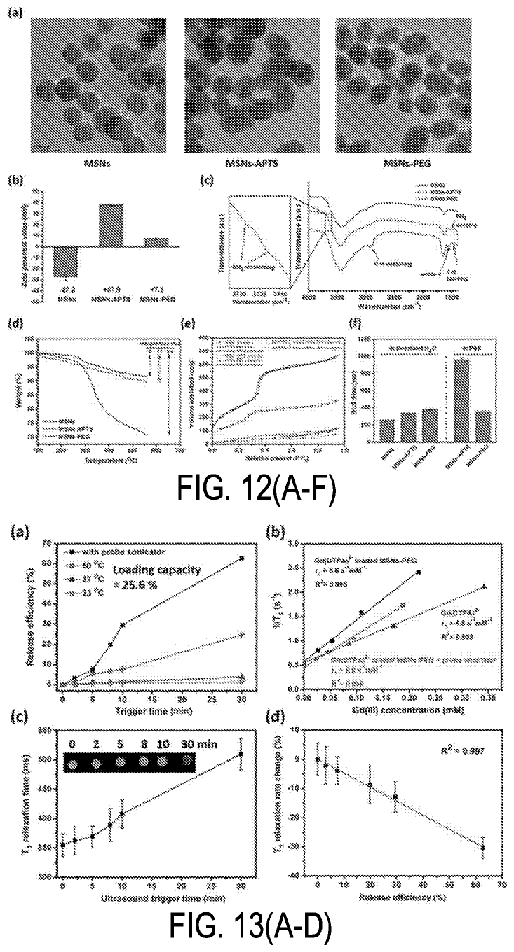

The morphology and diameters of nano-particles were characterized by transmission electron microscopy (TEM, Tecnai T12) with an operating voltage of 120 kV. MSNs or MSNs-APTS were dispersed in ethanol at a low concentration (0.1 mg/mL). The suspension (10 μL) of the nanoparticles was dropped onto a 200 mesh carbon-coated copper grid and dried at room temperature. The dynamic light-scattering (DLS) size and zeta potential values of nanoparticles were determined by a laser particle analyzer LPA-3100 at room temperature (23° C.). MSNs showed the characteristic zeta potential of −27.2 mV in deionized H₂O at pH 7, which was shifted to +37.9 mV after APTS modification (FIG. 12B). After PEGylation, the zeta potential dropped to +7.3 mV, which was the result of charge screening by the formed amide bonds. The functional groups on the surface of MSNs were characterized by Fourier transform infrared spectroscopy (FT-IR, JASCO FT/IR-420) spectrometer in the range of 4000-400 cm⁻¹. The successful modification was supported by the new absorption peaks at v=1512 cm⁻¹ (N—H bending) and the two bands at v=3717 cm⁻¹ and v=3727 cm⁻¹ (N—H stretching) in the FT-IR spectrum. The successful APTS modification was supported by the new absorption peaks at v=1512 cm⁻¹ (N—H bending) and the two bands 3717 cm⁻¹ and v=3727 cm⁻¹ (N—H stretching) in the FT-IR spectrum.

PEGylation was confirmed by the new absorption peak at v=1555 cm⁻¹ (amide II), supporting the formation of secondary amide bond. Stronger absorption peaks at v=1464 cm⁻¹ (C—H bending), and v=2880 cm⁻¹ and v=2927 cm⁻¹ (C—H stretching) of MSNs-PEG compared with MSNs-APTS also supported the successful PEGylation. Thermogravimetric analysis (TGA) was carried out using a PerkinElmer Pyris Diamond TG/DTA under air flow (200 mL/min). MSNs, MSNs-APTS, and MSNs-PEG (5-10 mg) were loaded in aluminum pans, and the data were recorded during a temperature scan from 30 to 550° C. at a scan rate of 10° C./min. The plotted values are normalized to the weight at 100° C. An empty aluminum pan was used as a reference. The weight loss of MSNs, MSNs-APTS, and MSNs-PEG were 8%, 11%, and 29%, respectively, confirming the presence of organic matter in MSNs-APTS and MSNs-PEG (FIG. 12D). The surface area, pore diameter, and pore volume of MSNs, MSNs-APTS, MSNs-PEG, or Gd(DTPA)²⁻-loaded MSNs-PEG were determined by N₂ adsorption-desorption isotherm measurement at 77 K (Autosorb-iQ, Quantachrome Instruments). Nanoparticles were degassed at 120° C. for 20 h before the measurement. The surface area and pore diameter distribution of the nanoparticles were determined by Brunauer-Emmett-Teller (BET) and Barrett-Joyner-Halenda (BJH) methods. The loading capacity and release efficiency of Gd(DTPA)²⁻ from MSNs-PEG were quantified by inductively coupled plasma optical emission spectrometry (ICP-OES) using a Shimadzu ICPE-9000. The calibration curve was created from 0 to 10 ppm of gadolinium in 2% HNO₃.

### Ultrasound-Stimulated Release of Gd(DTPA)²⁻ by a Probe Sonicator

Gd(DTPA)²⁻-loaded MSNs-PEG solution (3 mg/mL) was prepared in deionized H₂O in an Eppendorf tube. The tip of the probe sonicator (VCX 130, Sonics & Materials, Inc., Newtown, Conn.) was placed in the center of the solution. The ultrasound parameter was set with the frequency of 20 kHz and output power of 21 W (power density: 75 W/cm²). After various ultrasound stimulation time durations (2, 5, 8, 10, or 30 min) with the probe sonicator, the solution was centrifuged (14000 rpm, 10 min) to separate the particles and the supernatant containing released Gd(DTPA)²⁻. The supernatant and pellet were collected for further quantification of Gd(DTPA)²⁻ loading capacity and release efficiency by ICP-OES.

Quantification of Loading Capacity of Gd(DTPA)²⁻ in MSNs-PEG and Release Efficiency of Gd(DTPA)²⁻ after Ultrasound or HIFU Stimulation

Gd(DTPA)²⁻-loaded MSNs-PEG solution stimulated by ultrasound or HIFU was centrifuged (7830 rpm, 15 min) to separate the pellet and the supernatant. The particle-containing pellet was dispersed in 10 mL of aqua regia at 95° C. for 12 h to be fully digested into powder. The resulting powder was then dissolved and diluted by 2% HNO₃. The supernatant containing the released Gd(DTPA)²⁻ was diluted by 2% HNO₃. The concentration of Gd ions was measured by ICP-OES and quantified based on the Gd ion calibration curve (0, 0.01, 0.05, 0.1, 0.5, 1, 5, and 10 ppm). The definition of loading capacity is (mass of Gd(DTPA)²⁻ loaded in pores/mass of MSNs-PEG)×100%. The definition of release efficiency is (mass of released Gd(DTPA)²⁻/mass of Gd(DTPA)²⁻ loaded in pores)×100%.

### The Preparation of Agarose Phantom

An agarose gel “phantom” (i.e., test object) was prepared and used as the sample holder for Gd(DTPA)²⁻-loaded MSNs-PEG during MRgHIFU experiments. The concentration of the agarose used was 3.5 wt %. First, deionized H₂O (500 mL) was added into a 1000 mL flask. Agarose powder (17.5 g) was then added slowly to the flask during vigorous stirring. The solution was heated to boiling and maintained at that temperature for 5 min. Subsequently, the hot solution was poured into a plastic container with a diameter of 10 cm and the sample wells were molded by glass test tubes with a diameter of 1.3 cm. Finally, the solution was cooled to 4° C. for the gel formation.

### The Preparation of the Mixture of Methyl Cellulose and Concentrated Milk Gel

To prepare a mixture of methylcellulose gel (2.5 wt %) and milk (v/v=1/1), methylcellulose (1.25 g) was slowly added to 15 mL of boiling water in a flask under vigorous stirring to dissolve the powder. After the mixture was stirred for 3 min, 10 mL of room-temperature water and 25 mL of concentrated milk were rapidly added to the solution and mixed until the mixture was homogeneous. The solution was then cooled at 4° C. overnight to complete the gelation process.

### MRI-Guided High-Intensity Focused Ultrasound (MRgHIFU) Simulated Release of Gd(DTPA)²⁻

All MRgHIFU experiments were conducted using a research HIFU system (Image Guided Therapy, Bordeaux, France) integrated with a whole-body 3 T MRI scanner (Prisma, Siemens Healthineers, Erlangen, Germany). The HIFU system had a 128-element transducer array with a diameter of 9 cm, frequency of 1 MHz, a focal point of 1×1×7 mm³ in size, and a peak electrical power output of 1200 W. The electrical power output used ranged from 9 to 290 W. Gd(DTPA)²⁻-loaded MSNs-PEG dispersed in deionized H₂O (1 mg/mL, 3 mL), methylcellulose gel (2.5 wt %, 3 mL), or methylcellulose gel (2.5 wt %)/milk mixture (v/v=1/1) (3 mL) were placed in sample wells (1.3×1.3×5 cm³) in the agarose phantom (10×10×11.5 cm³). The agarose phantom was placed on top of the HIFU transducer, which was secured on the patient table of the 3 T MRI scanner. Through both mechanical and electronic steering of the HIFU transducer, the focal point was placed at the center of the sample well.

The samples were stimulated by HIFU at a fixed electrical power level (74 W, power density: 7400 W/cm²) with different durations (3, 5, or 10 cycles of 1 min), or at different electrical power levels (9, 74, or 290 W, power density: 900, 7400, or 29000 W/cm²) for three cycles of 1 min. The cooling period between each cycle was 10 s. T₁-weighted MR images were acquired before and after the HIFU stimulation using a 3D turbo-spin-echo protocol (see section T₁-weighted and T₂-weighted Images and T₁ and T₂ Mapping below) to compare the image intensity. The ΔT₁-weighted images were obtained by subtracting post-T₁-weighted images from pre-T₁-weighted images. The temperature of the solution during the HIFU stimulation was measured by a 2D gradient-echo MRI temperature mapping sequence every 1.8 s. To quantify the released amount of Gd(DTPA)²⁻, the HIFU-stimulated water-suspended samples were removed from the phantom and spun down to separate the pellet and supernatant. The particle-containing pellet was dispersed in 10 mL of aqua regia at 95° C. for 12 h to be fully digested into powder. The powder was then dissolved and diluted by 2% HNO₃. The supernatant containing the released Gd(DTPA)²⁻ was diluted by 2% HNO₃. The concentration of Gd ions was measured by ICP-OES.

T₁-Weighted and T₂-Weighted Images and T₁ and T₂ Mapping

T₁-weighted and T₂-weighted images of water-suspended Gd(DTPA)²⁻-loaded MSNs-PEG before and after the stimulation by a probe sonicator or MRgHIFU were acquired using a 3 T MRI scanner (Prisma, Siemens Healthineers, Erlangen, Germany) with gradient-echo or turbo-spin-echo (TSE) protocols respectively. The 1 mL samples with and without HIFU stimulation were mixed with 3 mL of methylcellulose (2.5 wt %) in 15 mL Falcon plastic tubes placed in a water bath. Acquisition parameters for gradient-echo T₁-weighted images were as follows: field of view (FOV)=350×350×60 mm³; matrix size=256×256×20; echo time (TE)=1.89 ms; repetition time (TR)=20 ms; flip angle=30°. Acquisition parameters for TSE T₂-weighted images were the following: FOV=350×350×60 mm³; matrix size=256×256×20; TE=118 ms; TR=8 s; flip angle=90°.

Parameters for the T₁ mapping protocol were as follows: inversion-recovery TSE sequence; FOV=350×350×60 mm³; matrix size=256×256×20; TE=13 ms; TR=8 s; inversion times (TIs)=50, 100, 200, 300, 500, 750, 1000, 1500, 2500 ms; excitation pulse flip angle=90°; inversion pulse flip angle=180°. Parameters for the T₂ mapping protocol were the following: multiple-TE TSE sequence; FOV=350×350×60 mm³; matrix size=256×256×20; TEs=12, 24, 35, 47, 59, 83, 94, 118 ms; TR=8 s; excitation pulse flip angle=90°; refocusing pulse flip angle=180°. T₁ and T₂ were calculated using a monoexponential fitting algorithm.

T₁ and T₂ Relaxivity (r₁ and r₂) Measurement

Different concentrations of Gd(DTPA)²⁻, Gd(DTPA)²⁻-loaded MSNs-PEG, or ultrasound or HIFU-stimulated Gd(DTPA)²⁻-loaded MSNs-PEG were mixed with 2.5 wt % methylcellulose. T₁ and T₂ relaxation times were acquired by the 3 T MRI scanner using the above inversion-recovery TSE sequence and multiple-TE TSE sequence, respectively. r₁ or r₂ were calculated as the ratio of 1/T₁ or 1/T₂ to the concentration of Gd(III) determined by ICP-OES.

### Molecular Weight Measurement of PEG by Matrix-Assisted Laser Desorption/Ionization Time-of-Flight Mass Spectrometry (MALDI-TOF MS)

HOOC-PEG-COOH (average Mn=2000 Da) dissolved in 3 mL of deionized H₂O (3 mg/mL) was added to the sample well in the agarose phantom. HIFU stimulation was focused at the center of the sample solution. After being stimulated for three cycles of 1 min or two cycles of 5 min (1 MHz, 74 W), the samples were collected and diluted (1.8 mg/mL) for the MALDI-TOF measurement. Sodium trifluoroacetate in deionized H₂O (10 mg/mL) was prepared as a cationizing agent. Dithranol dissolved in THF (20 mg/mL) was used as a matrix. PEG sample solution (5 μL), matrix solution (15 μL), and the cationizing agent (0.5 μL) were mixed well in an Eppendorf tube. A 1 μL portion of the mixture was spotted on a stainless steel target plate. Data were acquired in a reflection mode using a Bruker Ultraflex MALDI TOF/TOF mass spectrometer with the accelerating voltage at 25 kV.

### Ex Vivo MRgHIFU-Stimulated Gd(DTPA)²⁻ Release and MRI Contrast Change

The ex vivo experiments were conducted using the research HIFU system (Image Guided Therapy, Bordeaux, France) integrated with the whole-body 3 T MRI scanner (Prisma, Siemens Healthineers, Erlangen, Germany). The same HIFU system also had an 8-element annular transducer array with a diameter of 25 mm, frequency of 2.5 MHz, a focal point 0.7×0.7×3 mm³ in size, and a peak electrical power output of 200 W. Gd(DTPA)²⁻-loaded MSNs-PEG (0.5 mg) dispersed in 2.5% methylcellulose gel (0.5 mL) were injected in a sample of boneless chicken breast tissue (3×6 cm²) that was about 1 cm thick. The HIFU transducer was placed under the chicken breast tissue sample and secured on the patient table of the 3 T MRI scanner. The HIFU focal point was positioned in the chicken breast tissue close to the sample injection site, and the sample was stimulated with HIFU for 3 cycles of 3 min (8 W). Control groups including the samples (0.5 mg of Gd(DTPA)²⁻-loaded MSNs-PEG in 0.5 mL of 2.5% methylcellulose gel) injected into chicken breast tissue without HIFU stimulation and 2.5% methylcellulose gel (0.5 mL) injected into chicken breast tissue with HIFU stimulation (2 cycles of 3 min) were also included. T₁-weighted MR images were acquired before and after each HIFU stimulation cycle using the turbo-spin-echo protocol. The difference in T₁-weighted image intensity was obtained by subtracting post-T₁-weighted images from pre-T₁-weighted images, resulting in ΔT₁-weighted images. The temperatures of the chicken breast tissue during the HIFU stimulation were measured by a 2D gradient-echo MRI temperature mapping protocol before and right after the stimulation.

### Design of Mechanically Sensitive PEG-Functionalized Mesoporous Silica Nanoparticles

The nanoparticles may release cargo molecules by HIFU stimulation under MRI guidance. The FDA-approved contrast agent gadolinium-diethylenetriamine pentaacetic acid (Gd(DTPA)²⁻) and its counterions meglumines (i.e., gadopentetate dimeglumine: Magnevist) were chosen as the cargo for this example. The PEG in this example is an FDA-approved polymer that has been clinically used for pharmaceutical formulations. PEGylated nanoparticles exhibit the stealth effect because their interactions with the reticular-endothelial system are reduced, thus prolonging their circulation time and enhancing their uptake in tumor tissues via the enhanced permeability and retention (EPR) effect.

PEG also improves the colloidal stability of the nanoparticles. In this design, conjugated PEG seals the pores of the MSNs and exposes them only when stimulated by HIFU, where vigorous vibration and/or cleavage of PEG are induced. The entrapped Gd(DTPA)²⁻ that is released by externally controlled HIFU stimulation causes T₁-weighted MRI contrast changes. Therefore, MRI can be used to characterize the amount of HIFU-stimulated cargo release from the MSNs. Most importantly, this HIFU-stimulated cargo release does not require heating, and may be utilized with no heating or low levels of heating, opening up an opportunity for drug delivery when temperature increase is not practical or not desired.

The MSN nanocarriers were synthesized by a sol-gel reaction in the presence of cationic surfactant templates. The obtained nano-particles were 91.6±15.1 nm in diameter, and possessed an MCM-41 type structure. The inner and outer surfaces of MSNs were first functionalized with amine groups using (3-aminopropyl)triethoxysilane (APTS) for further coupling (designed as MSNs-APTS). Then, the outer surface of MSNs-APTS was coated with a dicarboxylic acid-terminated polyethylene glycol (HOOC-PEG-COOH, average Mn=2000 Da), which is designated as MSNs-PEG. On the outer surface, HOOC-PEG-COOH was conjugated to those amine groups by a standard 1-ethyl-3-(3-diethylaminopropyl) carbodiimide hydrochloride (EDC-HCl) and N-hydroxysulfosuccinimide sodium salt (sulfo-NHS) coupling reaction through amide bond formation (designated as MSNs-PEG). From transmission electron microscopy (TEM) images, the amine functionalization and PEGylation did not change the morphology or mesoporous structures of the nanoparticles (FIG. 12A).

The PEG coating covering the pores of MSNs-APTS can be clearly observed on the outer surface of the nanoparticles (FIG. 12A). PEG is an FDA approved polymer that is widely used in the field of biomedical research, especially used as excipients or as a carrier in different pharmaceutical formulations. However, to the best of our knowledge, this is the first work to apply PEGs themselves as the gatekeepers of MSNs that act on command by an external stimulus, ultrasound. Only when ultrasound is applied which induces the cleavage of PEG chains can the pores of MSNs be opened and, most importantly, the entrapped cargo molecules be released (FIG. 1). This strategy demonstrated that a cargo can be controlled released from MSNs-PEG without heating bulk environment, and thus the occurrence of hyperthermia can be avoided.

Dicarboxylic acid-terminated PEG (average Mₙ 2000 Da) was covalently conjugated to the surface of the MSNs-APTS by a standard coupling reaction between the carboxylic groups of the polymers and the amines at the surface of the MSNs-APTS. The nanoparticles grafted with PEG are designed as MSNs-PEG. Specifically, the carboxylic acids of PEG were activated by 1-ethyl-3-(3-diethylaminopropyl) carbodiimide hydrochloride (EDC-HCl) and N-hydroxysulfosuccinimide sodium salt (sulfo-NHS). To well cover the pore, the activated PEGs were added dropwise to the MSNs-APTS, allowing both ends of the activated PEG chains link to the amines on the surface of MSNs-APTS though amide bond formation and create a U-shaped gatekeeper.

The successful amine functionalization and PEG conjugation to MSNs was confirmed by zeta potential, Fourier transform-infrared (FT-IR), thermogravimetric analysis (TGA), N₂ adsorption/desorption isotherm, and dynamic light scattering (DLS) measurement after each step. MSNs showed the characteristic zeta potential of −27.2 mV in deionized H₂O at pH 7, which was shifted to +37.9 mV after APTS modification (FIG. 12B). After PEGs grafting, the zeta potential further dropped to +7.3 mV, which is the result of charge screening by the amide bonds formed between the PEGs and the MSNs-APTS. After modifying with APTS, the FT-IR spectrum shows new absorption peaks at ν=1512 cm⁻¹ (N—H bending) from APTS (FIG. 12C). Two bands at ν=3717 cm⁻¹ and ν=3727 cm⁻¹ arise from N—H stretching vibrations also support the presence of primary amines. The conjugation of PEG to the surface of MSNs-APTS through secondary amide bond formation (amide II) was confirmed by a new characteristic amide absorption at ν=1555 cm⁻¹. Stronger C—H bending at ν=1464 cm⁻¹ of MSNs-PEG than those of MSNs-APTS, and the characteristic peaks of C—H stretching at ν=2880 cm⁻¹ and ν=2927 cm⁻¹, also support the successful grafting of PEG to MSNs-APTS.

The grafting weight of APTS and PEG were determined by TGA. After heated to 550° C. in air, the weight loss of MSNs, MSNs-APTS, and MSNs-PEG was 8, 11, and 29%, respectively, confirming the presence of organic matter in MSNs-APTS and MSNs-PEG, from where the grafting weight of APTS and PEG to the surface of MSNs are 3 and 18%, respectively (FIG. 12D). Brunauer-Emmett-Teller (BET) surface area, total pore volume, and average pore diameter of MSNs, MSNs-APTS and MSNs-PEG were analyzed from the N₂ adsorption/desorption isotherms (FIG. 12E). Compare to MSNs who has 2.9 nm in average pore diameter, 1045 m²/g in BET surface area, and 1.04 cc/g in pore volume, MSNs-APTS and MSNs-PEG show smaller BET surface area (595 and 108 m²/g, respectively), total pore volume (0.52 and 0.19 cc/g, respectively), and average pore diameter (2.4 and 2.0 nm, respectively) (Table 1).

The decrease in BET surface area, total pore volume, and average pore diameter can be explained by the coverage to the surface of nanoparticles and the blockage to the pore openings after the sequential grafting of APTS and PEG to MSNs. Another support for successful grafting of APTS and PEG was acquired from the increased DLS size of MSNs (282.1±8.6 nm), MSNs-APTS (337.5±5.7 nm), and MSNs-PEG (384.2±4.1 nm) in deionized H₂O (FIG. 12F).

The DLS sizes of both MSNs-APTS and MSNs-PEG in phosphate-buffered saline (PBS) were also evaluated. Without PEGs, MSNs-APTS in PBS showed a significantly increased DLS size (959.2±27.9 nm) compared to that of in H₂O, indicating the severe aggregation of nanoparticles due to high ionic strength of PBS. On the other hand, MSNs-PEG dispersed in PBS (356.6±1.4 nm) has a well similar size compared to that of in H₂O. MSNs-PEG and MSNs-APTS in PBS were kept undisturbed for 30 min, 1 day, and 3 days at room temperature. Substantial aggregation of MSNs-APTS was observed within 30 min, whereas MSNs-PEG remained well-suspended even after 3 days. The improved colloidal stability of PEGylated nanoparticles can be explained by the blocking of surface contact between nanoparticles by surface PEG and preventing aggregation of the nanoparticles by such steric repulsion.

### The Loading of Gd-DTPA²⁻ in MSNs-PEG and Thermal Stability

The loading of Gd-(DTPA)²⁻ was carried out by soaking MSNs-APTS in a Gd(DTPA)²⁻ solution (500 mM) and stirring overnight. The Gd(DTPA)²⁻-loaded MSNs-APTS were then conjugated with HOOC-PEG-COOH (Mw=2000 Da) to seal the pores. The mixture reacted overnight and was washed thoroughly with deionized H₂O to remove the unloaded or surface Gd-(DTPA)²⁻. To evaluate the loading capacity of Gd(DTPA)²⁻ in MSNs-PEG, Gd(DTPA)²⁻-loaded MSNs-PEG was digested by aqua regia at 95° C. The amount of Gd ions in the resulting powder was then dissolved by 2% HNO₃ and measured by inductively coupled plasma optical emission spectrometry (ICP-OES). The loading capacity of Gd(DTPA)²⁻, defined as (mass of Gd(DTPA)²⁻ loaded in pores/mass of MSNs)×100%, was calculated to be 24.1±1.2%. This high loading of Gd(DTPA)²⁻ in the pores resulted in the decreased BET surface area and the decreased total pore volume (FIG. 13A).

Because of both the small size of Gd(DTPA)²⁻ molecules (ca. 0.8 nm) and the electrostatic interaction between the negatively charged Gd(DTPA)²⁻ and the positively charged pore walls, the Gd(DTPA)²⁻ may easily diffuse into the pores and stick to the pore wall. Another type of positively charged amine, N-trimethoxysilylpropyl-N,N,N-trimethylammonium (TA), was attached to the pore walls of MSNs (designated as MSNs-TA) to compare the loading of Gd(DTPA)²⁻ with that of MSNs-APTS. MSNs-TA showed lower loading capacity of Gd(DTPA)²⁻ (11.0±0.5%), implying that the steric hindrance of the bulky trimethyl groups in TA weakened the electrostatic interaction between the quaternary amine and Gd(DTPA)²⁻.

To cover multiple pores, both carboxylic acids of HOOC-PEG-COOH were conjugated to amines on the surface of MSNs-APTS through stepwise conjugation method and formed U-shaped PEG caps. To confirm the formation of U-shaped PEG, its Gd(DTPA)²⁻ loading capacity after thorough washing was compared to that of the control group capped with monocarboxylic acid-terminated PEG, where the PEG can be attached to MSNs-APTS only at one end, leaving the other end extended into the solvent. As expected, the stretched form PEG cap showed lower Gd(DTPA)²⁻ loading capacity (18.4%), confirming that the stepwise conjugation method that attaches both ends of HOOC-PEG-COOH to MSNs-APTS improves the pore-capping capability. In addition to the geometry of PEG caps, the distance between PEG caps and the surface of MSNs-APTS was also another factor to optimize.

Here, the capping capabilities of HOOC-PEG-COOH with higher molecular weight (Mn=6500 Da and 25000 Da) were explored, with which the Gd(DTPA)²⁻ loading capacities were 19.0% and 19.1%, respectively. The lower Gd(DTPA)²⁻ loading capacities using these longer PEG caps compared to that of the shorter PEG (Mn=2000 Da) may be attributed to the larger gap space between the U-shaped cap chain and the pore opening, allowing more Gd(DTPA)²⁻ molecules to diffuse out during the washing steps.

The stability of the PEG cap in aqueous solution at both room temperature (23° C.) and body temperature (37° C.) was examined. Gd(DTPA)²⁻-loaded MSNs-PEG (3 mg/mL) was suspended in deionized H₂O in Eppendorf tubes. Each tube was immersed in a water bath at room temperature (23° C.) or 37° C. for 2, 5, 8, 10, or 30 min. Negligible leakage of Gd(DTPA)²⁻ molecules was observed at room temperature. At 37° C., only 3.8% of Gd(DTPA)²⁻ was released over a period of 30 min, showing that PEG was tight enough to cap the pores and remained stable on the MSN's surface even at a physiological temperature.

Controlled Release of Gd(DTPA)²⁻ from Mechanically Sensitive MSNs-PEG by a Probe Sonicator

To examine the ultrasound responsiveness of the PEG cap, a probe sonicator (VCX 130, Sonics & Materials, Inc., Newtown, Conn.) was used as an ultrasound source for a proof-of-concept experiment. Gd(DTPA)²⁻-loaded MSNs-PEG suspended in deionized H₂O was stimulated with the probe sonicator (20 kHz, power density: 75 W/cm²) for 2, 5, 8, 10, or 30 min. (FIG. 13A). The Gd(DTPA)²⁻ release efficiency was defined as (mass of released Gd(DTPA)²⁻/mass of Gd-(DTPA)²⁻ loaded in pores)×100%, where the mass of released Gd(DTPA)²⁻ was quantified by ICP-OES. The amount of released Gd(DTPA)²⁻ increased with the sonication time, where 30 min of sonication led to a release efficiency of 62%. This result confirms that ultrasonication uncapped the PEG-covered pores of MSNs and that the amount of released cargo was controllable by the sonication time.

The temperatures of the samples measured with a thermometer immediately after sonication were 31, 42, 47, 49, and 51° C. after the 2, 5, 8, 10, and 30 min stimulation, respectively, by the probe sonicator. To measure the effect of only heat on the release, a control experiment was carried out using a water bath at 50° C. Over a period of 30 min 24% of the Gd(DTPA)²⁻ was released, which is less than half of that (62%) triggered with sonication and heating to a similar temperature (FIG. 13A). This result shows that although heat can cause partial release of Gd(DTPA)²⁻ molecules, the released amount was relatively small because PEG is not degradable at 50° C. Mechanical effects of ultrasound, such as shock waves created during cavitation, induce PEG rupture and thus efficiently release the entrapped cargo. Cavitation causes a rapid compression and rarefaction of the liquid. On a molecular level it causes a rapid motion of small molecules (e.g., solvent molecules and Gd(DTPA)²⁻) that the polymer in the solvent cannot follow, thereby generating friction, increasing strain, and eventually leading to bond rupture. The TEM image of the nanoparticles after being exposed to ultrasound and high temperature (50° C.) for 30 min shows that the mesoporous structure was intact. The release of Gd(DTPA)²⁻ was primarily through opening of the pores stimulated by the rupture of the mechanically sensitive PEG gatekeeper rather than by the destruction of the mesoporous silica structure.

Although temperature itself may accelerate molecular diffusion, ultrasound with the additional mechanical effects such as shock waves created during cavitation, is thought to induce the polymer rupture which in turn may be the predominant factor involved in the release of Gd(DTPA)²⁻. Indeed, cavitation may cause a rapid compression with subsequent expansion of the liquid, where on a molecular level, implies a rapid motion of small molecules (e.g., solvent molecules, Gd(DTPA)²⁻) to which the polymer in the solvent cannot follow. Thus, friction is generated, strain is increased, and eventually, bond rupture mostly at the midpoint of the polymer chains occurs.

To exclude the possibility that ultrasound and high temperature may destruct the mesoporous structure of nanoparticles and thus release Gd(DTPA)²⁻, the structure of MSNs-PEG after 30 min of sonication was analyzed by TEM. The TEM image shows that the mesoporous silica structure was intact without any damage and the diameters of the particles didn't change after exposing to ultrasound reaching the temperature around 50° C. The result also confirms that the release of Gd(DTPA)²⁻ is through the removal of the PEG gatekeepers rather than the destruction of nanoparticles.

To investigate if the Gd(DTPA)²⁻ loaded MSNs-PEG can serve as a good T₁-weighted MRI contrast agent, different concentrations of Gd(DTPA)²⁻ loaded MSNs-PEG were dispersed in the deionized H₂O and their T₁ relaxation times were measured with a 3 T MRI instrument. T₁ relaxivity (r₁) of the Gd(DTPA)²⁻ loaded MSNs-PEG was calculated through the ratio of 1/T₁ to the concentration of gadolinium (Gd (III)), where the concentration of Gd (III) in the solution was determined by ICP-OES. r₁ of the Gd(DTPA)²⁻ loaded MSNs-PEG (8.6 s⁻¹mM⁻¹) was found to be 1.9 times higher than the r₁ of free Gd(DTPA)²⁻ (4.5 s⁻¹ mM⁻¹) (FIG. 13B).

The increased relaxivity of the nanoparticulate Gd(DTPA)²⁻ when they were loaded inside the pores of MSNs-PEG can be explained by their reduced tumbling rate. And the different relaxivities of the Gd(DTPA)²⁻ between when it was loaded in MSNs-PEG and it is free in the solution generates the T₁-weighted image contrast difference which could be used to easily follow the release of DTPA²⁻ from MSNs-PEG. This also supports that Gd(DTPA)²⁻-loaded MSNs-PEG itself can be a good candidate for T₁ contrast enhancement, generating stronger signal than an equal concentration of Gd(DTPA)²⁻. This may be beneficial for further cancer diagnostic as some of gadolinium-based contrast agents are retained in the body, and FDA have just announced this year that the use of them requires new class warnings. By using Gd(DTPA)²⁻-loaded MSNs-PEG as a new type of contrast agent, we may have a chance to reduce the total amount of Gd(DTPA)²⁻ needed for MRI diagnostic.

We attempted to examine if the MR imaging intensity change with the help of contrast agents could be a checkpoint of the cargo released from the nanoparticles qualitatively and/or quantitatively. According to FIG. 13A, the amount of released Gd(DTPA)²⁻ increased with the sonication time. Thus, we first investigated the change in T₁ relaxation time as a function of sonication time. The Gd(DTPA)²⁻ loaded MSNs-PEG were triggered with the probe sonicator for 2, 5, 8, 10, or 30 min, then the MRI measurement was carried out to get T₁ relaxation times of each sample. Before sonication, T₁ relaxation time is 353 ms; after 30 min of sonication, T₁ relaxation time significantly increased to 500 ms (43% change) (FIG. 13C). This positive correlation between T₁ relaxation times and the sonication time can be explained as follows: when applying ultrasound, the pores were permanently opened through PEG degradation or alternatingly opened between each PEG vibration, resulting in the release of Gd(DTPA)²⁻.

Compared to the loaded Gd(DTPA)²⁻, the released Gd(DTPA)²⁻ have faster tumbling rate and a longer T₁ relaxation time. That is, the more released Gd(DTPA)²⁻ will lead to the longer T₁ relaxation time. The significant change in T₁ relaxation time was clearly observed from T₁-weighted images: the images became dimmer under longer sonication time, suggesting more released Gd(DTPA)²⁻ with the enhanced tumbling rate appeared in the system (FIG. 13C). This finding implies that by examining the T₁ relaxation time and/or the intensity of T₁-weighted images acquired from MRI, we can potentially determine qualitatively and/or quantitatively: (1) if the PEG caps are opened, (2) the amount of Gd(DTPA)²⁻ released from the MSNs-PEG, and (3) the amount of therapeutics released from the MSNs-PEG if they are co-loaded with Gd(DTPA)²⁻.

The fact that ultrasound can trigger the release of Gd(DTPA)²⁻ are also supported by the reduced relaxivity (6.6 s⁻¹mM⁻¹) for Gd(DTPA)²⁻ loaded MSNs-PEG after 30 min of sonication by the probe sonicator (FIG. 13B). The reduced relaxivity suggested some of the loaded Gd-DTPA²⁻ were released so that their average tumbling rate became closer to the free Gd(DTPA)²⁻. T₁-weighted images corresponding to each sample were also presented, where higher Gd (III) concentration led to brighter image.

In addition to T₁, T₂ relaxation times also correlated well with sonication time: T₂ relaxation time increased with sonication time, which is also attributed to the faster tumbling rate of released Gd(DTPA)²⁻. Longer sonication time resulting in more Gd(DTPA)²⁻ released gave rise to less dark T₂-weighted images. This implies the potential of using this platform to conduct the dual-module imaging on the basis of both T₁ and T₂, which could further improve the diagnostic efficacy.

MRI-Guided HIFU-Stimulated Release of Gd(DTPA)²⁻ and MRI Contrast Change

The stimulated release experiments were carried out using a research HIFU system (Image Guided Therapy, Bordeaux, France) integrated with a whole-body 3 T MRI scanner (Prisma, Siemens Healthineers, Erlangen, Germany). MRI was used to guide the HIFU stimulation, monitor the temperature during the stimulation, and pinpoint the release of Gd(DTPA)²⁻ in real time. The HIFU transducer has a 128-element array with a frequency of 1 MHz and a peak electrical power output of 1200 W. The mechanical and electronic steering capabilities of the HIFU system can precisely steer the 1×1×7 mm3 cigar-shaped HIFU focal point in three dimensions.

In the fields of MRI and ultrasound (including HIFU), it is customary to first use tissue-mimicking “phantoms” to evaluate feasibility, train operators, optimize protocols, and characterize technical performance of MRgHIFU technology before studying animal or human subjects. Tissue-mimicking phantoms use materials designed to mimic pertinent properties of biological tissues and thus are used in preclinical research as an alternative to ex vivo tissues and organs. The advantages of using tissue-mimicking phantoms include superior availability and shelf life, high structural uniformity, and customizability. Several HIFU tissue-mimicking phantom formulations such as agar, gelatin, and polyacrylamide with additives to adjust their thermal and acoustic properties to be comparable to human soft tissues have been reported.

For example, the acoustic attenuation and speed of sound of tissue-mimicking phantoms may be adjusted via the addition of silicon dioxide particles, concentrated milk, bovine serum albumin, corn syrup, glass beads, intralipid, graphite, or n-propanol. Because of the similar convection and diffusion properties to that of extracellular space around tumor tissues, agarose phantoms are commonly used for ultrasound-stimulated drug delivery studies. For example, other researchers have used an agarose-based phantom with a thermosensitive indicator to study the spatial drug delivery profile using ultrasound-induced mild hyperthermia.

In addition to the above-mentioned phantoms, researchers in the community of MRI or ultrasound also commonly study nanoparticle samples in water or gel for initial demonstration of their new techniques. In this example, we followed the methodology in the community of MRI or ultrasound by using an agarose phantom as the sample holder to mimic aqueous tissues and by placing nanoparticle samples in water for sample recycling and further quantification or in methylcellulose gel to mimic a tissue scaffold.

Concentrated milk was selected as the primary attenuation component in this work for its widespread availability, a high attenuation coefficient (˜0.8 dB/cm/MHz) with a speed of sound that is typical of biological fluids (1547 m/s), and its previous uses in water-based phantoms. The agarose phantom used in this work was 10 cm in diameter and contained sample wells that were 1.3 cm in diameter and 5 cm in height. Agarose is sturdy and does not liquefy below 65° C. The ultrasound waves penetrate through agarose without being absorbed because of its low acoustic attenuation coefficient. The agarose phantom was placed on the HIFU transducer on the patient bed in the MRI scanner. Two samples of water-suspended Gd(DTPA)²⁻-loaded MSNs-PEG, three samples of Gd(DTPA)²⁻-loaded MSNs-PEG mixed in methylcellulose (2.5 wt %) and concentrated milk (v/v=1/1)(designated as gel/milk), and gel/milk itself were placed in the six sample wells in the agarose phantom. The phantom on the transducer was moved into the MRI scanner for further MRgHIFU experiments.

The aqueous suspension of Gd(DTPA)²⁻-loaded MSNs-PEG (sample 1) was stimulated with HIFU (3 cycles of 1 min, 74 W). T₁-weighted turbo-spin-echo images of the entire agarose phantom were acquired before and after the HIFU stimulation, termed pre- and post-T₁-weighted images, respectively. The post-T₁-weighted image of sample 1 was darker than the pre-T₁-weighted image. To clearly depict the change in MR image intensity, T₁-weighted images were processed by subtracting the post-T₁-weighted image from the pre-T₁-weighted image to generate the ΔT₁-weighted image. The HIFU-stimulated sample 1 exhibited a bright signal in the ΔT₁-weighted image. The homogeneous brightness in the well was the result of diffusion of the released Gd(DTPA)²⁻ in the well. The unstimulated control groups, including Gd(DTPA)²⁻-loaded MSNs-PEG in water (control 2), Gd(DTPA)²⁻-loaded MSNs-PEG in gel/milk (controls 3-5), and gel/milk itself (control 6), showed negligible changes between pre- and post-T₁-weighted images as expected. The agarose phantom background (control 7) also showed no image change. In the entire 3-D ΔT₁-weighted image of the agarose phantom containing the sample wells, only the HIFU-stimulated sample 1 strikingly showed an intense signal.

The amount of Gd(DTPA)²⁻ released by HIFU stimulation was quantified by ICP-OES. HIFU-stimulated sample 1 had the released amount of Gd(DTPA)²⁻ of 0.47 μmole of Gd(DTPA)²⁻/mg of MSNs-PEG. The change in T₁-weighted image intensity, evaluated as [(pre-T₁-weighted image intensity−post-T₁-weighted image intensity)/pre-T₁-weighted image intensity]×100%, was calculated to be 39% for the HIFU-stimulated sample 1. This result confirmed that HIFU-stimulated Gd(DTPA)²⁻ release occurred primarily in the well into which the HIFU was focused, with negligible effect on the other wells.

The above discussion emphasized the magnitude of the change in the T₁-weighted image intensity. Initially it seemed surprising that Gd(DTPA)²⁻ release caused a decrease in the intensity. However, the decrease in T₁-weighted image intensity when the encapsulated Gd(DTPA)²⁻ was released from the pores of the nanoparticles can be explained by the Solomon-Bloembergen-Morgan theory that describes the parameters affecting T₁ caused by a given Gd(III)-based contrast agent.

According to the theory, T₁ decreases when the rotational correlation time (τᵣ) increases (i.e., decreased rotation or tumbling rate of the Gd(III) contrast agent). Numerous studies have shown that Gd(III)-based contrast agents bonded to bulky proteins, nanoparticles, or peptides tumble more slowly and thus increase the contrast. In the case of Gd(DTPA)2-loaded in the MSN's pores, electrostatic interactions with the positively charged pore wall of the bulky MSNs-PEG decreased the tumbling rate and thus enhanced r₁ to 8.6 s⁻¹mM⁻¹. After the HIFU stimulation (3 cycles of 1 min, 74 W), where 26% of the loaded Gd(DTPA)²⁻ was released, r₁ of Gd(DTPA)²⁻-loaded MSNs-PEG was reduced (6.9 s⁻¹mM⁻¹) compared to that before HIFU stimulation, similar to what was found by ultrasound stimulation with a probe sonicator.

No changes in either r₁ or r₂ of Gd(DTPA)²⁻ itself were observed after HIFU or the probe sonicator stimulation under the same conditions, confirming that the structure of the Gd(DTPA)²⁻ molecule was not changed by HIFU stimulation. By using this nanoparticulated Gd(DTPA)²⁻, the total amount of Gd(DTPA)²⁻ required to enhance T₁-weighted MR image contrast to the same level as that of free Gd(DTPA)²⁻ can be reduced. This may be beneficial for avoiding accumulation of gadolinium-based contrast agents in the body over repeated contrast-enhanced examinations, which was described in a warning by the FDA in 2018.

### Mechanisms of HIFU-Stimulated Gd(DTPA)²⁻ Release

To study if the activation of the PEG cap by HIFU and the ensuing release of Gd(DTPA)²⁻ was caused by heat, mechanical forces, or both, the temperature change caused by HIFU stimulation was measured. The increase in temperature was monitored by dynamic MRI temperature mapping acquired using a 2D gradient-echo protocol (TE=20 ms, TR=30 ms, spatial resolution of 1×1×3 mm³, and temporal resolution of 2.8 s). The maximum temperature rise of water-suspended Gd(DTPA)²⁻-loaded MSNs-PEG was only 4° C. during the 1 min HIFU stimulation period, and the temperature returned to the starting temperature (20° C. room temperature) shortly after HIFU stopped. The minimal temperature rise can be explained by the low sound attenuation coefficient of water; negligible sound energy was absorbed, and thus very little heat was generated during HIFU stimulation.

Even at this low temperature (less than 24° C.), HIFU can stimulate 0.47 μmole of Gd(DTPA)²⁻/mg of MSNs-PEG release from the pores of MSNs-PEG. In contrast, the temperature rose by 11° C. after 1 min of HIFU stimulation of the sample mixed in the gel/milk mixture that has a higher sound attenuation coefficient. Water therefore served as an ideal medium to demonstrate the mechanical sensitivity of PEG in the absence of appreciable temperature changes. The mechanical effects of HIFU allowed cargos to be released with minimal temperature increase, unlike the case where probe sonication was used.

To confirm the mechanical sensitivity of the polymer cap, PEG cleavage by HIFU at room temperature was examined by matrix-assisted laser desorption ionization-time-of-flight mass spectrometry (MALDI-TOF MS). The peaks from intact PEG were located around m/z=2000. After HIFU stimulation (3 cycles of 1 min, 1 MHz, 74 W), small peaks around m/z=1000 appeared. The signal intensity of those peaks increased with longer HIFU stimulation duration (2 cycles of 5 min). The intensity ratios of the fragment peaks around m/z=1000 to m/z=2000 (defined as [the highest intensity of the peaks around m/z=1000/the highest intensity of the peaks around m/z=2000]) were 0.11 (without HIFU), 0.20 (after 3 cycles of 1 min HIFU), and 0.71 (after 2 cycles of 5 min HIFU).

The PEG cleavage was likely caused by HIFU-induced cavitation and is consistent with previous studies showing that ultrasonic chain scission occurred around the center of the polymer chain. PEG itself may also accelerate polymer degradation because hydrophilic PEG enhances the penetration of water and thus the rate of hydrolysis. Further evidence of PEG degradation by HIFU was the decrease in colloidal stability of MSNs-PEG. Aggregation of the HIFU-stimulated nanoparticles implies that the PEG on the surface was degraded.

MRI-Guided Controlled Release of Gd(DTPA)²⁻ from Mechanically Sensitive MSNs-PEG by HIFU

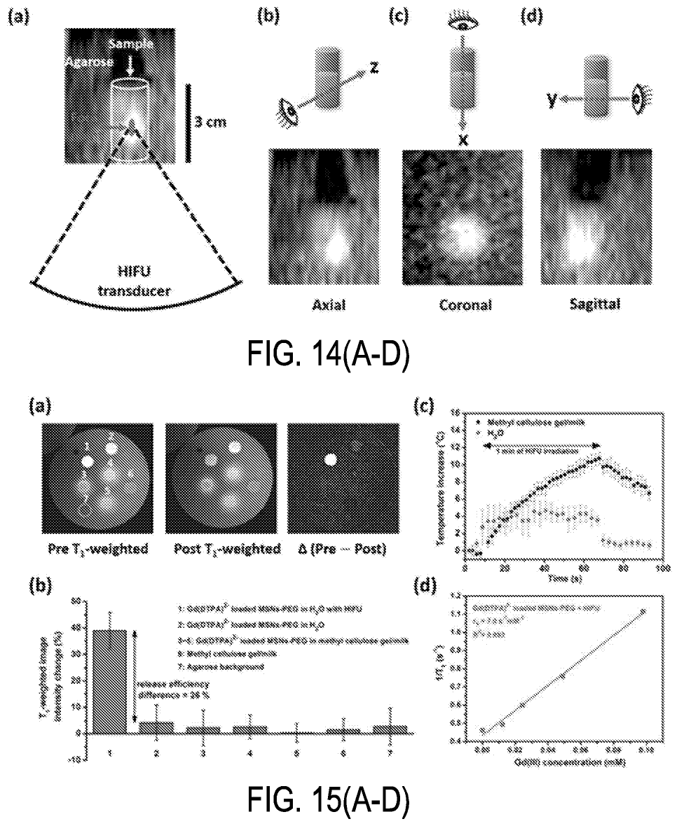

The basic principle of high-intensity focused ultrasound (HIFU) can be illustrated as using a magnifier to collect sunlight and burn a small hole in a leaf, where HIFU instead utilizes a transducer (like the magnifier) to direct and focus sound energy (like sunlight) to a millimeter-sized area. In this case, the substance in that small focal spot have a much more intense interaction with HIFU than that away from the focal spot, and thus changes physically or chemically. On the basis of this, we explored if we could combine magnetic resonance guided HIFU (MRgHIFU) with the developed MSNs-PEG to achieve the controlled release of Gd(DTPA)²⁻. Tissue mimicking the water-cooled HIFU transducer was placed under an agarose phantom located on a patient bed of the MRI scanner. Gd(DTPA)²⁻ loaded MSNs-PEG were dispersed in the mixture of 5 wt % of methyl cellulose and concentrated milk (v/v=1/1) and placed in a well created in the agarose phantom as shown in FIG. 14A.

The samples were irradiated with a HIFU beam focused to a cigar-shaped region with dimensions of 1×1×7 mm³ and irradiated for 3 cycles of 1 min. To monitor the MR contrast change, T₁-weighted images of the samples were acquired before (pre) and after (post) the HIFU stimulation. A more conspicuous way to illustrate T₁ MR contrast change was subtracting the T₁-weighted images acquired after HIFU stimulation from those acquired before HIFU stimulation (Δ (pre−post)). Three orientations (axial, coronal, and sagittal) of those Δ (pre−post) T₁-weighted images were showed in FIG. 14B-14D, where the bright white region was located surrounding the focal point in the three orientations. The bright white region appearing after the image subtraction indicates that the T₁-weighted image intensity (contrast) of the sample before HIFU stimulation was stronger than that after HIFU stimulation.

In T₁-weighted images, dimmer image is resulted from longer T1 relaxation time, and in this case, from the Gd(DTPA)²⁻ exiting from MSNs-PEG after HIFU stimulation. More specifically, the released Gd(DTPA)²⁻ has a faster tumbling rate in the absence of the electrostatic interaction between the positively-charged pore wall of MSNs-PEG and the negatively-charged Gd(DTPA)²⁻ molecules; the faster tumbling Gd(DTPA)²⁻ molecules then lead to a longer T₁ relaxation time of protons compared to the Gd(DTPA)²⁻ loaded in the MSNs-PEG. Because of the highly-focused nature of HIFU, the triggered release of Gd(DTPA)²⁻ only occurred around the focal point, confining the image-changing area to a clear-cut region on the order of dozens of mm³. In view of this, we could qualitatively reveal the time window and the three-dimensional location of the release of Gd(DTPA)²⁻ through simply monitoring the change in T₁-weighted image intensity.

The next step was to both qualitatively and quantitatively determine the amount of released Gd(DTPA)²⁻ from MSNs-PEG after HIFU stimulation. Gd(DTPA)²⁻ loaded MSNs-PEG was dispersed in deionized H₂O so that the released Gd(DTPA)²⁻ can be easily separated from nanoparticles and quantified with ICP-OES. FIG. 15A shows the T₁-weighted images of the water-suspended Gd(DTPA)²⁻ loaded MSNs-PEG (sample 1) before and after HIFU stimulation (3 cycles of 1 min) and their Δ (pre−post) T₁-weighted image. Similar to the change observed from the sample mixed in the gel/milk mixture, the contrast of T₁-weighted image of the water-suspended sample became weaker after irradiated with HIFU. The control samples showing negligible change in T₁-weighted image contrast included: water-suspended Gd(DTPA)²⁻ loaded MSNs-PEG without HIFU stimulation (control 2), Gd(DTPA)²⁻ loaded MSNs-PEG in the gel/milk mixture without HIFU stimulation (controls 3 to 5), and the gel/milk itself (control 6).

The agarose phantom background (control 7) also didn't obviously change its contrast after HIFU stimulation. Amazingly, the HIFU irradiated sample exhibited significant brightness in the Δ (pre−post) T₁-weighted image that is distinct from other control samples. This notable change in the contrast can also be clearly observed from the three-dimensional illustration of the Δ (pre−post) T₁-weighted image. Rather than forming a clear-cut bright region close to the focal point like the sample mixed in gel/milk showed in FIG. 14B to 14D, the whole sample well filled with the water-suspended sample turned intensely white. This nearly homogeneous contrast change in water after HIFU stimulation can be explained by the higher diffusivity of the released Gd(DTPA)²⁻ in water compared to that in the mixture of gel/milk.

These results provided the qualitative evidence to support that: (1) MSNs-PEG was able to prevent small Gd(DTPA)²⁻ molecules from leaking in both aqueous solution and gel/milk, (2) Gd(DTPA)²⁻ was successfully released from MSNs-PEG into both aqueous solution and gel/milk under HIFU stimulation, and (3) HIFU would only trigger the release of Gd(DTPA)²⁻ from the sample located close to its focal point, with little interference to other surrounding samples. The percentage change of T₁-weighted image intensity of the sample and controls in FIG. 15A were calculated as [(pre T₁-weighted image intensity−post T₁-weighted image intensity)/pre T₁-weighted image intensity]×100%, and the release efficiency of the sample after HIFU was found to be 26% by ICP-OES (FIG. 15B).

This quantitative evidence confirms the amount of Gd(DTPA)²⁻ released from the nanoparticles after HIFU stimulation. A positive correlation between the released amount of Gd(DTPA)²⁻ and the T₁-weighted image intensity change—26% of Gd(DTPA)²⁻ amount to 35% of change in T₁-weighted image contrast—suggests that by examining the change in T₁-weighted images acquired from MRI, we can potentially determine qualitatively and/or quantitatively: (1) if the PEG caps are opened and (2) the amount of Gd(DTPA)²⁻ released from MSNs-PEG. If we further co-load therapeutics with Gd(DTPA)²⁻ in MSNs-PEG, the amount of released therapeutics could be calibrated and derived from the release amount of Gd(DTPA)²⁻, holding great promise for meeting criterion (3).

As previously mentioned, MSNs-PEG is mainly sensitive to mechanical forces instead of heat, and thus is a good candidate for realizing drug delivery without hyperthermia. Water-suspended Gd(DTPA)²⁻-loaded MSNs-PEG (sample 1 in FIG. 15A) was used to prove this concept as water has a very low sound attenuation coefficient. Negligible sound energy will be absorbed by water, and thus less temperature rise will occur. Near real-time temperature during the HIFU stimulation of 1 min was acquired from MRI using a 2D phase imaging protocol. As expected, the maximal temperature rise (4° C.) of sample 1 dispersed in water during the HIFU stimulation was much less compared to that of the sample mixed in the methyl cellulose gel and milk mixture (11° C.) (FIG. 15C).

The highest temperature of the whole system in water only reached 27° C. during HIFU stimulation, even lower than 37° C. physiological temperature, demonstrating the cargo release capability of MSNs-PEG under such low bulk temperature. Indeed, we can also induce the release of Gd(DTPA)²⁻ by bulk heating as discussed in FIG. 13A; however, 37° C. bulk heating only contributed 3.8% of the Gd(DTPA)²⁻ release, and even under 50° C., it still requires 30 min to release a certain amount of Gd(DTPA)²⁻. The release of Gd(DTPA)²⁻ after HIFU stimulation was also supported by its reduced r₁ value (7.0 s⁻¹mM⁻¹) compared to that of Gd(DTPA)²⁻ loaded MSNs-PEG as showed in FIG. 13B (FIG. 15D). T₁-weighted images corresponding to the sample after HIFU were also presented, where higher Gd (III) concentration gave brighter image.

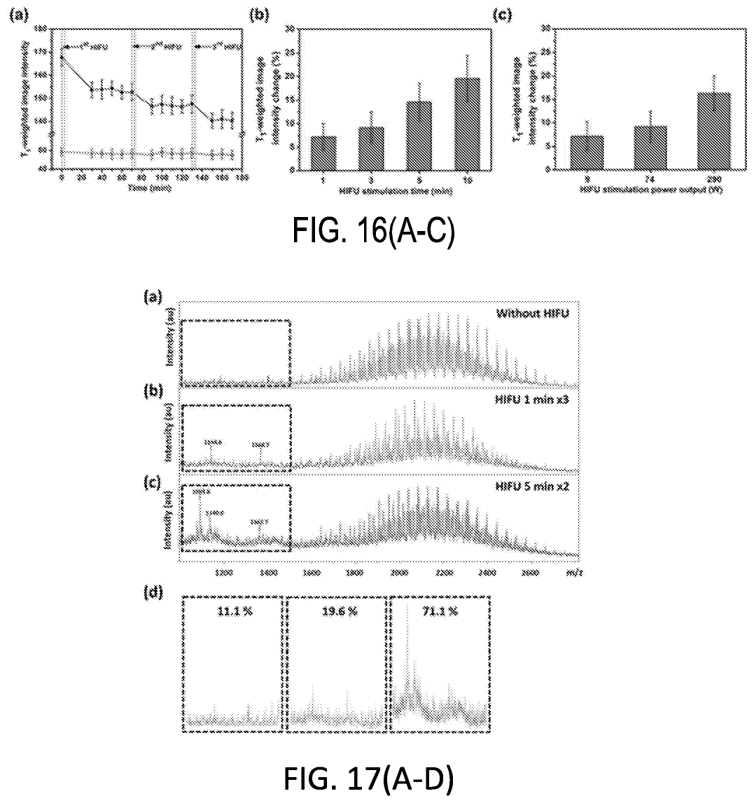

To demonstrate the potential of adjusting the dose of released cargo, three types of release studies were carried out as followed: (i) multiple HIFU stimulation each with the same length of time, (ii) a single exposure to HIFU with different stimulation times, and (iii) a single exposure to HIFU at different power outputs. FIG. 16A shows the schematic representation of study (i). Gd(DTPA)²⁻ loaded MSNs-PEG were dispersed in 2 mL of methyl cellulose/milk (3 mg/mL) and filled in the sample wells in an agarose phantom. After the first cycle of HIFU stimulation, the T₁-weighted image intensity kept decreasing over a period of 30 min, suggesting that the release of Gd(DTPA)²⁻ mostly occurred within this 30 min, and then it reached equilibrium until the next HIFU stimulation.

Similarly, after the second cycle and the third cycle of HIFU stimulation, the T₁-weighted image intensity decreased within a certain period of time and reached equilibrium. Study (i) implies that the cargo release from MSNs-PEG could be controlled temporally to achieve the desired amount of released cargo within the specific time window. This may be especially beneficial for treating diseases such as type 1 diabetes that requires multiple daily dosing. With the drug delivery strategy developed in this study, only one injection to patient may be needed. By simply applying multiple HIFU stimulation, the therapeutics could be released in situ stepwise and finally reach the amount in its therapeutic window

In study (ii), Gd(DTPA)²⁻ loaded MSNs-PEG dispersed in methyl cellulose gel/milk were irradiated with HIFU at a power output of 74 W for 1, 3, 5, or 10 min. Tt-weighted images of the samples were acquired before and after the stimulation. FIG. 16B shows the change in T₁-weighted image intensity (Δ (pre−post)) of the samples irradiated with those time lengths. 1 min of stimulation resulted in 9% decrease of the T₁-weighted image intensity, and 10%, 13%, and 16% decrease of the T₁-weighted image intensity was achieved after 3, 5, and 10 min of HIFU stimulation, respectively. The change in T₁-weighted image intensity grew with HIFU stimulation time, which is reasonable because the more decrease in T₁-weighted image intensity indicates more Gd(DTPA)²⁻ being released by longer HIFU stimulation time.

In study (iii), Gd(DTPA)²⁻ loaded MSNs-PEG dispersed in methyl cellulose gel/milk were irradiated with HIFU for a fixed length of 3 min at different power outputs: 9 W, 74 W, and 290 W. FIG. 16C shows that the T₁-weighted image intensity decreased 5%, 10%, and 13% respectively with 9 W, 74 W, and 290 W HIFU power output. With increasing HIFU power, the change in T₁-weighted intensity became more pronounced, indicating more Gd(DTPA)²⁻ was released. This can be explained by the fact that a stronger cavitation is generated at a higher acoustic intensity. Studies (ii) and (iii) imply that the amount of Gd(DTPA)²⁻ released from MSNs-PEG can be controlled by adjusting HIFU parameters such as stimulation length and power output.

To justify the release mechanism discussed above, degradation of PEG after stimulation with HIFU was confirmed with matrix assisted laser desorption ionization-time of flight mass spectrometry (MALDI-TOF MS) at room temperature. Without HIFU stimulation, the only peak showed up is located around m/z=2000, which is the peak of PEG itself. After stimulation with HIFU (3 cycles of 1 min, 1 MHz, 74 W), some peaks around m/z=1000 appeared. The signal intensity of those peaks significantly increased after even harsher HIFU treatment (2 cycles of 5 min). The intensity ratios of the peaks around m/z=1000 to m/z=2000 defined as (the highest intensity of the peaks around m/z=1000/the highest intensity of the peaks around m/z=2000) were determined to be 11%, 20%, and 71% for the samples without HIFU, after 3 cycles of 1 min HIFU and 2 cycles of 5 min HIFU. Interestingly, the intensity ratio of the peaks around m/z=2000 to m/z=1000 rose with the extent of HIFU stimulation, which is consistent with the previous studies suggesting that ultrasonic chain scission usually occurs around the center of the polymer chain.

The cleavage of PEG, especially at a focused spot, could be explained by the cavitation effect induced by HIFU stimulation. In addition to the previous discussion, the bond rupture in PEG may also be caused by the localized high temperature and high pressure created when bubbles rapidly collapse during rarefaction. This polymer degradation may be accelerated by PEG itself, because hydrophilic PEG can enhance the penetration of water and thus the rate of hydrolysis.

Controlled Release of Model Drug from Mechanically Sensitive MSNs-PEG by Probe Sonicator

To demonstrate that MSNs-PEG can be a potential theranostic nanoplatform for future cell and animal studies, rhodamine 6G (R6G) was used as a model drug molecule based on its similar size (c.a. 1.4 nm) to some of anticancer drugs. Also, since R6G possesses positive charge opposite to Gd(DTPA)²⁻, the release study of R6G helps us to investigate the applicability of MSNs-PEG to cargos with different charges. The loading of R6G was carried out by soaking MSNs-APTS in 1 mM of the R6G solution for 24 h. After this, the pores of MSNs-APTS were capped with PEG for another 24 h. Then, the solution was thoroughly washed with H₂O to remove the unloaded R6G and the excess EDC/NHS. R6G loaded MSNs-PEG was dispersed in 1 mL of deionized H₂O in each Eppendorf tube. The controlled release of R6G was then demonstrated using the probe sonicator as a trigger. The probe sonicator was inserted into each tube and the particle solutions were sonicated (20 kHz and 52 mW) for 2, 5, 8, 10, 30, or 60 min.

Without sonication, negligible R6G leakage was found over a period of 1 hour, showing successful capping of PEG for R6G molecules. When triggered by the probe sonicator, R6G started to release after 10 min of sonication, resulting in 4 folds and 9 folds release amount of R6G after 20 min and 60 min of sonication, respectively. On the contrary, the MSNs without PEG caps showed significant R6G leakage, suggesting the necessity of capping the pores of MSNs with PEGs.

The release of R6G was also studied in PBS solution to simulate physiological environment. Similarly to in H₂O, negligible R6G leakage was found in PBS over a period of 1 hour without sonication and the R6G were successfully triggered released by the probe sonicator. The MSNs without PEG caps also showed significant R6G leakage in 20 min, again proving an evidence of the good capping capability of PEGs even in PBS solution.

The ultimate goal for drug delivery is to reduce the side-effects of drugs. Although many drug delivery have been developed, a majority of them are responsive to internal stimuli such as pH, redox, or enzymes, and it is almost impossible to precisely control the location and time the therapeutics are released from the drug carriers, needless to say the desired dosage of therapeutics within its therapeutic window.

If the temperature increases too high and leads to hyperthermia, other concerns may need to be considered including the possibility of tumor metastasis and the challenges in keeping the area of interested at a stable temperature that would not cause detrimental effect to nearby tissues. To meet this unmet need, some embodiments of the present disclosure provide advantages of mechanized silica nanoparticles (MSNs with caps) with MRgHIFU technique (e.g., FIGS. 1 and 10) to create stimuli-responsive compositions that respond, for example, to HIFU and do not increase the temperature of the bulk environment. To this effect, a capping agent, such as PEG, was chosen that exhibits stability at physiological temperature, and can only be operated under HIFU, even at lower temperature. For biomedical application, different from the ablation therapy widely-used with HIFU, this minimal or no-heating cargo release strategy will not induce hyperthermia, providing alternative approach for chemotherapy in cancer treatment without the risk of metastasis.

In clinical cancer treatment, to make decisions of the next treatment in time, an opportunely assessment of therapeutic response is critical. However, there is still limited work directly applying their ultrasound-responsive drug delivery to MRgHIFU. In this work, the combined application of MRgHIFU with MSNs-PEG can potentially report the therapeutics level in tumors in real time. For example, co-encapsulation of Gd(DTPA)²⁻ and anticancer drugs within MSNs-PEG may facilitate analysis of release kinetics of drugs. A good correlation observed between the release amount of Gd(DTPA)²⁻ with the change in T₁-weighted image intensity and/or T₁ relaxation time implies that the amount of drugs co-released with Gd(DTPA)²⁻ from MSNs-PEG can also be probed and monitored in situ, opening up the opportunity of dose painting for imageable nanoplatform in combination with MRI. Most importantly, T₁-maps can reveal strong intratumoral inhomogeneities and intertumoral variations, providing the drug delivery developed in this work another tool for personalized tumor therapy.

To confirm the PEG cleavage occurred in response to HIFU, PEG (average Mₙ 2000 Da) dissolved in deionized H₂O after HIFU stimulation was examined by matrix assisted laser desorption ionization-time of flight mass spectrometry (MALDI-TOF MS) at room temperature. Without HIFU, the only peak located around m/z=2000 was the peak of PEG itself (FIGS. 17A-D). After stimulated with HIFU (3 cycles of 1 min, 1 MHz, 74 W), small peaks around m/z=1000 showed up. The signal intensity of those peaks increased with harsher HIFU treatment (2 cycles of 5 min).

This polymer degradation may also be accelerated by PEG itself because hydrophilic PEG can enhance the penetration of water and thus the rate of hydrolysis. The degradation of PEG after the HIFU treatment was also evidenced by the colloidal stability of MSN-PEG. It was found that HIFU-treated MSNs-PEG was less stable than that of untreated MSNs-PEG in DI water, as the aggregation of the nanoparticles was observed 30 min after the treatment. The result implies that the PEG on the surface may be degraded after the HIFU treatment and the colloidal stability was thus decreased.

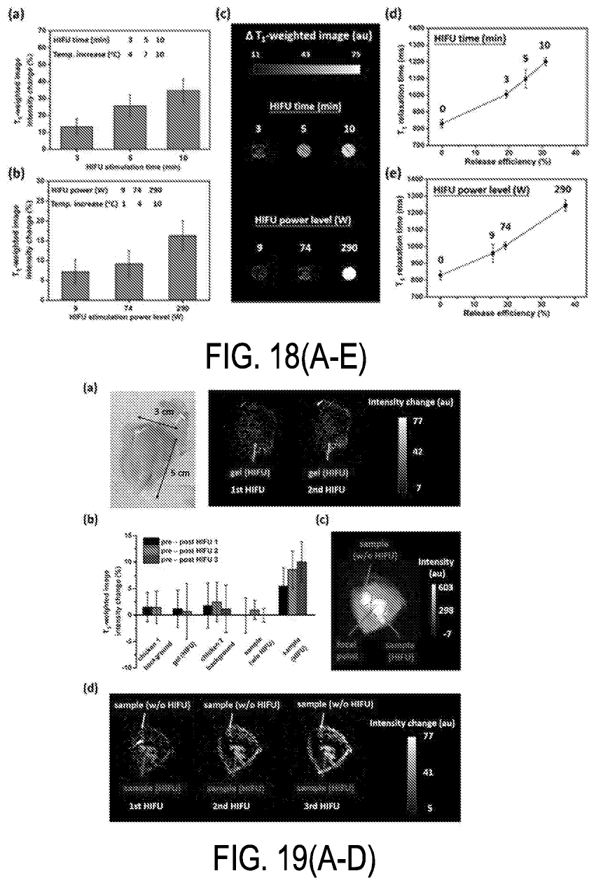

Next, we explored different HIFU stimulation times and power levels to operate the T₁-weighted image intensity and T₁ relaxation time. A total of three Gd(DTPA)²⁻-loaded MSNs-PEG samples dispersed in deionized H₂O were stimulated with HIFU at a power level of 74 W for 3, 5, or 10 min, respectively. The change in T₁-weighted image intensity were acquired before and after HIFU stimulations. 3 min of HIFU stimulation resulted in a 13% change in T₁-weighted image intensity (FIG. 18A). 26% and 35% change were achieved after 5 and 10 min of HIFU stimulations, respectively. Greater decrease in T₁-weighted image intensity with longer HIFU stimulation time implied more released Gd(DTPA)²⁻. Their Δ T₁-weighted images also showed that the stronger Δ T₁-weighted image intensity came from the longer HIFU stimulation time (FIG. 18C).

The other three Gd(DTPA)²⁻-loaded MSNs-PEG samples dispersed in deionized H₂O were stimulated with HIFU for a fixed duration (3 min) but at different power levels (9, 74, and 290 W). At 9 W and 74 W, the change in T₁-weighted image intensity was 5% and 13%, respectively (FIG. 18B). Amazingly, 60% change in T₁-weighted image intensity was achieved at 290 W HIFU power level. This can be explained by the strong cavitation generated at such high acoustic intensity, leading to much more released Gd(DTPA)²⁻. Their Δ T₁-weighted images also showed the same trend as discussed above and the pronounced intensity at 290 W HIFU power level was observed (FIG. 18C). The temperature increase during those HIFU stimulations was monitored by the near real-time temperature mapping. During 3 min of HIFU stimulation at 74 W, the temperature only increased 4° C. Even during 5 min or 10 min HIFU stimulation, the temperature only went up 7° C. or 10° C. (FIG. 18A). Stimulated with HIFU power at 9 W or 290 W for 3 min, the temperature increased 1° C. or 10° C., respectively (FIG. 18C). We further examine the structure of MSNs-PEG after the treatment by HIFU. The TEM image shows that the mesoporous structure was intact after being exposed to HIFU for 3 min.

This demonstrates that the release of Gd(DTPA)²⁻ was through opening of the pores that are controlled by the mechanically sensitive PEG gatekeeper rather than the destruction of mesoporous silica structure, similar to that of triggered-release by an ultrasound probe. Similar release experiments were also done using gel/milk mixed Gd(DTPA)²⁻-loaded MSNs-PEG. Additionally, multiple HIFU stimulations (3 cycles of 1 min) was done. After the first cycle of HIFU, the T₁-weighted image intensity kept decreasing over a period of 30 min, suggesting the release of Gd(DTPA)²⁻ mostly occurred within this 30 min, and then it reached equilibrium until the next HIFU stimulation. Similarly, after the second and the third cycles of HIFU stimulation, the T₁-weighted image intensity decreased within a certain period of time and then reached equilibrium. This implied that the release of Gd(DTPA)²⁻ could be controlled temporally to achieve the desired release amount within a specific time window.

One of the main goal in this work is to find the correlation of T₁ relaxation time with the release efficiency of Gd(DTPA)²⁻. Thus, we further analyzed those HIFU-stimulated samples in FIGS. 18A and 18B by ICP-OES to quantify the release amount of Gd(DTPA)²⁻ Excitingly a positive correlation between the T₁ relaxation time and the release efficiency of Gd(DTPA)²⁻ was found (FIG. 18D and FIG. 18E), which was well-linked to the Solomon-Bloembergen-Morgan theory. This finding implied that (1) the release amount of Gd(DTPA)²⁻ from MSNs-PEG can be well controlled by adjusting HIFU stimulation time or power level and (2) the release amount of Gd(DTPA)²⁻ can be directly derived from the T₁ relaxation time.

### Dose and Temporal Control of Released Gd(DTPA)²⁻ by MRgHIFU Stimulation and Power Level

The effects of exposure time and HIFU power levels on the amount of Gd(DTPA)²⁻ released were measured based on the change of the T₁-weighted image intensity. Gd(DTPA)²⁻-loaded MSNs-PEG samples were dispersed in deionized H₂O and stimulated with HIFU at a fixed power level (74 W) for 3, 5, or 10 min. The T₁-weighted intensities decreased by 13%, 26%, and 35%, respectively, and the corresponding ΔT₁-weighted images increased in brightness with increasing HIFU stimulation time. Similarly, the dependence of the Gd(DTPA)²⁻ release on HIFU power levels was also measured. Three samples of water-suspended Gd(DTPA)²⁻-loaded MSNs-PEG were stimulated with HIFU for 3 min at power levels of 9, 74, and 290 W. The changes in T₁-weighted image intensity were 5%, 13%, and 60%, respectively. The Δ T₁-weighted image of the sample stimulated at 290 W showed strong signal changes which can be explained by strong cavitation and PEG fragmentation caused by such high acoustic intensity. The temperature increases during these HIFU stimulations were monitored by dynamic MRI temperature mapping. During 3 min of HIFU stimulation at 74 W, the temperature increased by only 4° C. The temperature increased by 7° C. during a 5 min exposure and by 10° C. during 10 min of exposure. For the 3 min stimulations at different power levels, the temperature increases were 1° C. at 9 W and 10° C. at 290 W.

The correlation between the amount of Gd(DTPA)²⁻ released and T₁ was quantified. The T₁ relaxation times from the HIFU-stimulated samples were measured by MRI T₁ mapping, and the amount of Gd(DTPA)²⁻ released was measured by ICP-OES. Increased Gd(DTPA)²⁻ release from the pores of MSNs-PEG resulted in longer T₁ relaxation times. By adjusting the HIFU stimulation time or power level, the released amount of Gd(DTPA)²⁻ could be controlled and monitored from the T₁ relaxation time. Table 1a summarizes the Gd(DTPA)²⁻ release efficiency and temperature increase under various HIFU parameters (power levels and stimulation time). Similar results were found using probe sonication: T₁ relaxation times increased with increasing duration of ultrasonication and thus higher release efficiency of Gd(DTPA)²⁻.

TABLE 1a
Summary of Gd(DTPA)²⁻
release efficiency and
temperature increase using
various HIFU parameters.

| HUT-parameters | | |
|---|---|---|
| (power level, | Release | Temperature |
| stimulation time) | efficiency | increase |
| (9 W, 3 min) | 15.61% | 1° C. |
| (74 W, 3 min) | 19.29% | 4° C. |
| (74 W, 5 min) | 25.05% | 7° C. |
| (74 W, 10 min) | 31.01% | 10° C. |
| (290 W, 3 min) | 37.22% | 10° C. |

To demonstrate that HIFU can control the released dose of Gd(DTPA)²⁻ over time, Gd(DTPA)²⁻-loaded MSNs-PEG samples were mixed in gel/milk and stimulated with HIFU for various durations and at different power levels. Again, the greater the HIFU stimulation times and power levels, the greater the decrease in T₁-weighted image intensity. To control the release of Gd(DTPA)²⁻ over time, multiple cycles of HIFU stimulations were performed. After the first cycle of HIFU stimulation, the T₁-weighted image intensity decreased over a period of 30 min and leveled off. The second and third cycles of HIFU stimulation resulted in similar profiles. This sequence shows that the desired dose of released Gd(DTPA)²⁻ can be achieved within a specific time window by adjusting the ON and OFF times of HIFU stimulation. The structure of the MSNs-PEG was intact after HIFU stimulation for 3 min, suggesting that the release of Gd(DTPA)²⁻ was due to the opening of the pores gated by the mechanically sensitive PEG rather than by the destruction of the nanoparticle.

### Three-Dimensional Spatial Control of Released Gd(DTPA)²⁻ at the Focal Point of MRgHIFU Stimulation

To visualize the 3-D precision with which the Gd(DTPA)²⁻ cargo is released from the nanoparticles, Gd(DTPA)²⁻-loaded MSNs-PEG particles were mixed in a viscous gel to minimize diffusion of the Gd(DTPA)²⁻ molecules after release. The particles were homogeneously mixed in 2.5 wt % methyl-cellulose, and the sample was transferred into sample wells (1.3×1.3×5 cm³) molded in the agarose phantom. The well dimensions were large enough to allow the cigar-shaped HIFU focal point (1×1×7 mm³) to be positioned well within the well's interior.

Considering the release studies in the previous sections, three cycles of 1 min HIFU stimulation (1 MHz, power level: 74 W) were chosen to activate the release of Gd(DTPA)²⁻. The MRI was acquired with a turbo-spin-echo inversion-recovery protocol shortly after the HIFU stimulation, and cross sections of the Δ T₁-weighted images are shown in three orientations (FIG. 14B-D). A clearly defined region of image signal change appeared. The cigar-shaped red spot, the position with maximal image intensity change, pinpoints the HIFU focal point which was near the center of the sample well. The change of image intensity away from the focal point sharply decreased, showing that the majority of the Gd(DTPA)²⁻ was released at the focal point. The temperature increased 4° C. after the HIFU stimulation, again confirming that the release was primarily due to the mechanical responsiveness of the PEG cap.

### Ex Vivo HIFU-Stimulated Gd(DTPA)²⁻ Release and MR Image Change:

To demonstrate the potential of transferring our technology to preclinical and clinical in vivo studies in the future, 3-D spatial and dose-controlled cargo release in ex vivo chicken breast tissue samples was investigated. A 3×6 cm² piece of chicken breast was injected with methylcellulose gel containing Gd(DTPA)²⁻-loaded MSNs-PEG at two different locations and imaged before HIFU stimulation. The two injection sites were observable as bright regions in T₁-weighted images (chicken 1, FIG. 19A).

A HIFU transducer with an 8-element annular array and similar size (25 mm in diameter) as the chicken breast was used for MRgHIFU. The HIFU focal point was 0.7×0.7×3 mm³ in size, and the peak electrical power output of the HIFU transducer was 200 W. One of the injection sites was stimulated with HIFU for 3 cycles of 3 min (2.5 MHz, 8 W).

After 3 cycles of HIFU stimulation, a clearly confined region of intensity change close to the HIFU focal point was observed in 3-D space. Negligible changes in T₁-weighted image intensity were observed at the sample injection site without HIFU stimulation or in the background. These results confirm that MRgHIFU can achieve spatially selective stimulation for cargo release close to the HIFU focal point in ex vivo tissue. By using this technique, we can first visualize the presence of Gd(DTPA)²⁻-nanoparticles with MRI, stimulate with MRgHIFU at the focal point, and characterize the cargo release from the MRI contrast change.

A potentially challenging property of tissue is its acoustic energy-absorbing ability due to the abundance of proteins that may generate significant heat during HIFU stimulation and produce overall image contrast changes caused by the temperature increase. To examine if such temperature interference occurred, a control experiment was done by injecting methylcellulose gel into a 3×5 cm² sample of chicken breast. The gel injection site was stimulated by HIFU for 2 cycles of 3 min (2.5 MHz, 8 W). The temperature increase at the focal point measured by a 2D gradient-echo protocol was 10° C. A negligible change in T₁-weighted image intensity was observed showing that the temperature interference was not a confounder.

### Potential Biomedical Applications

It is contemplated that certain embodiments of the present disclosure may facilitate the transfer of cargo delivery techniques to solving real clinical problems such as cancer staging and treatment planning. In clinical cancer treatment, to set up the treatment plans in time, a timely assessment of therapeutic response is important. Another key challenge in cancer treatment is to examine if the drugs are delivered to the tumor tissue and not the healthy tissue. Those two challenges could be overcome by co-encapsulating therapeutics and imageable cargos such as Magnevist in MSNs-PEG. By monitoring the drug carriers under the guidance of MRI, the drug release will only be activated by HIFU when the drug carriers arrive at the tumor site and the drug release behavior can be observed in situ. The release amount of drugs may be acquired potentially from the good correlation between the release amount of Magnevist with the change in T₁-weighted image intensity and T₁ relaxation time found in certain embodiments of the present disclosure. In certain aspects, the release amount of therapeutics can be tuned by simply adjusting the HIFU parameters. In this case, the goal of precision medicine, defined as giving a precise dosage of drugs in a specific location at a controlled time, can be achieved. The HIFU-triggered PEG cleavage may be alternative strategy for overcoming the “PEG dilemma” in addition to pH-, redox-, or enzyme-triggered PEG cleavage.

Another advantage of certain aspects of the present disclosure is that no heat or minimal heat induces release of drugs to the target region. Thus, certain aspects of the present disclosure may (1) be more beneficial for treating diseases such as pancreatic cancer or liver cancer that need to avoid heat in the region (e.g., increases of greater than 5 degrees Celsius), and (2) will not induce hyperthermia which may reduce the risk of tumor metastasis.

To develop a theranostics nanoparticle on the basis of MRI, three general criteria were desired: (1) an enhanced contrast effect, (2) a large payload of therapeutics and contrast agents, and (3) a good correlation between the therapeutics and the imaging agents delivered. Indeed, most contrast agents are small and non-targeted compounds which tend to passively distribute into the interstitial space of tissues, resulting in insufficient contrast enhancement. Many studies thus took advantage of the EPR effect, conjugating the contrast agents to materials including nanoparticles, liposomes, polymers, and microbubbles, to passively target the tumor site.

Also, the resulting particulate contrast agents have the enhanced relaxivity as a result of reduced tumbling rates of the contrast agents, thereby improving the contrast signal. However, there exists two main challenges to meet criterion (3) when MRI contrast agents are directly conjugated to nanoplatforms. First, it would be difficult to have a direct correlation of the contrast change in response to the release of therapeutics. Second, the released therapeutics may stand a good chance to have a very different kinetics and biodistribution to that of the nanoparticulate MRI contrast agents, making it complicated to track the released therapeutics by MRI. In this study, MSNs especially provide an advantageous platform for MRI theranostics nanoparticle due to their high surface areas that can interact with contrast agents, allowing a large payload of contrast agents and therapeutics to be carried (achieving criterion (2)). Indeed, the negatively-charged Gd(DTPA)²⁻ loaded inside the MSNs-PEG with the positively-charged pore wall can have a strong electrostatic interaction resulting in a reduced tumbling rate, a short longitudinal relaxation time (T₁), and an enhanced contrast effect (achieving criterion (1)).

Some embodiments of the present disclosure provide minimal or no-heating cargo delivery strategies of which the cargo release can be triggered on command upon exposure to ultrasound waves. Two types of model cargos—Gd(DTPA)²⁻ (FDA approved MRI T₁ contrast agent, commercially known as Magnevist®) and R6G (similar size to some anticancer drugs)—was explored. A large amount of Gd(DTPA)²⁻ (26% of loading) loaded in polyethylene glycol (PEG) capped MSNs (MSNs-PEG) showed a better T₁ contrast enhancement compared to the one with the equal amount of free Gd(DTPA)²⁻ as the loaded Gd(DTPA)²⁻ has slower tumbling rate: the r₁ value of the loaded Gd(DTPA)²⁻ (8.6 s⁻¹mM⁻¹) is 1.9 times higher than that of free Gd(DTPA)²⁻ (4.5 s⁻¹mM⁻¹).

In view of this difference in magnetic resonance image (MRI) contrast enhancement, Gd(DTPA)²⁻ release from MSNs-PEG triggered by a probe sonicator and high intensity focused ultrasound (HIFU) was monitored based on the image intensity change. For example, the release efficiency of Gd(DTPA)²⁻ was 62% after 30 min of the probe sonicator trigger, corresponding to 31% of relaxation rate change. 26% of Gd(DTPA)²⁻ was released from MSNs-PEG in water after only 3 min of HIFU stimulation, corresponding to 39% of T₁-weighted image intensity change.

This approach is advantageous in several aspects. First, the PEG cap regulating the cargo release is itself biocompatible and effectively stabilizes the entire particles in physiological environment. Second, the release of Gd(DTPA)²⁻ from MSNs-PEG could be spatially and temporally controlled by a probe sonicator and HIFU. Third, the PEG cap is mainly responsive to mechanical force, and thus Gd(DTPA)²⁻ could be efficiently released from MSNs-PEG with the temperature rise in the bulk solution by no more than 5° C. after HIFU stimulation. Finally, the amount of Gd(DTPA)²⁻ released from MSNs-PEG could be regulated by: multiple sequential HIFU stimulations, HIFU stimulation time, and power output, and monitored by the intensity change in T₁-weighted image between pre- and post-HIFU stimulation. R6G was showed to be controlled released from MSNs-PEG after the treatment with probe sonicator, implying this cargo delivery system could be applied to the delivery of cargos with different sizes and charges. In view of those features, the present disclosure may be applicable to alternative personalized cancer therapy that allows the avoidance of hyperthermia thus preventing tumor metastasis, and quantitatively assess the drug release from T₁ contrast change.

### Example 2: Exemplary Materials, Methods of Fabrication, and Characterization of Silica Particles Configured with Thermo-Responsive Capping Agents as Described Herein

### Synthesis of Mesoporous Silica Nanoparticles (MSN)

0.25 g of CTAB and 875 μL of sodium hydroxide solution (2 M) were dissolved in 120 mL of water under stirring. The solution was heated at 80° C. for 30 minutes, followed by the addition of 1.2 mL of TEOS and 0.79 mL of ethyl acetate under vigorous stirring. Stirring was continued for 2 h at 80° C., and then the solution was allowed to cool to room temperature. The nanoparticles were collected by centrifugation (15 min at 7830 rpm), washed 3× with ethanol (3×30 mL) and dispersed in 20 mL ethanol for further use. Approximately 200 mg of MSN will be obtain in each batch. MSN was around 120 nm, and modified with amine group on the surface.

### Synthesis of MSN-Linking Agent Particles:

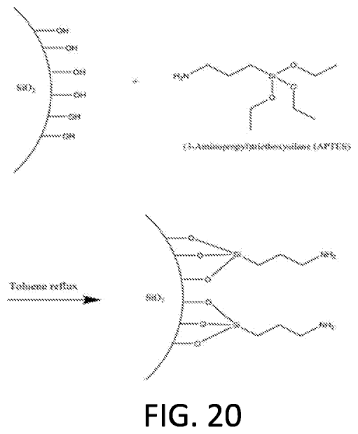

FIG. 20 is a schematic illustration of an example method for producing an amine-modified MSN. Around 200 mg of unfunctionalized MSNs is washed 2× with toluene (2×30 mL), and redispersed in 30 mL of dry toluene stirring in a flame-dried 50 mL round bottom flask under nitrogen. Then 120 μL of (3-Aminopropyl)triethoxysilane is added drop by drop and resulting mixture is refluxed in 110° C. oil bath under nitrogen overnight. The amine-modified MSNs is collected by centrifugation (10 min at 7830 rpm) and washed 3× with ethanol (3×30 mL). Product is redispersed in 20 mL of ethanol for further use.

APTES-functionalized MSNs dispersed in toluene were washed 2× with ethanol (2×30 mL). To extract the organic template from the pores, the nanoparticles were dispersed in 80 mL of an acidic ethanolic solution (EtOH:HCl(conc.)=90/10 (v/v)), refluxed for 1 hr, collected by centrifugation (10 min at 7830 rpm), and repeated above procedure one more time. The product was washed 2× with ethanol (2×30 mL) and stored in ethanol.

Amine modification was tried in two methods: co-condensation, which means condensing (3-Aminopropyl)triethoxysilane (APTES) in silica framework, and post-grafting, which is shown in FIG. 20. Delivery performance was tested separately.

In some embodiments, Gd(DTPA)²⁻ was attached. To attach Gd(DTPA)²⁻, three synthesis methods were tried: electrostatic attraction between negative charge of Gd(DTPA)²⁻ and positive charge amine on MSN, DTPA coupling by refluxing in DMSO followed by Gd³⁺ chelate and EDC/NHS reaction. EDC/NHS reaction had fewer steps and the highest yield. The Gd(DTPA)²⁻ amount on Gd(DTPA)²⁻ modified MSN (Gd-MSN) was measured by ICP-OES.

In one example, NH₂-MSN dispersed in ethanol was washed 3× with DI water (3×30 mL), then dispersed in HEPES buffer (pH=7.4) for future use. 1.2 mL of Gd(DTPA)²⁻ water solution (0.10 g/mL, pH=6.7) was mixed with 4.8 mL of MES buffer (100 mM, pH=6.0), 17.2 mg of EDC.HCl and 19 mg of sulfo-NHS, and the mixture was stirred for 20 min. 0.3, 0.6, 2.1, 3 mL of mixture were added to 5 mL of HEPES buffer with 60 mg NH₂-MSN dispersed, and stirred in room temperature for 24 h. Gd(DTPA)²⁻ modified MSNs (Gd-MSNs) products were washed 3× with DI water (3×30 mL) and labeled as sample 1 to sample 4 (S1, S2, S3, S4).

### Synthesis of MSN-PNIPAm Particles:

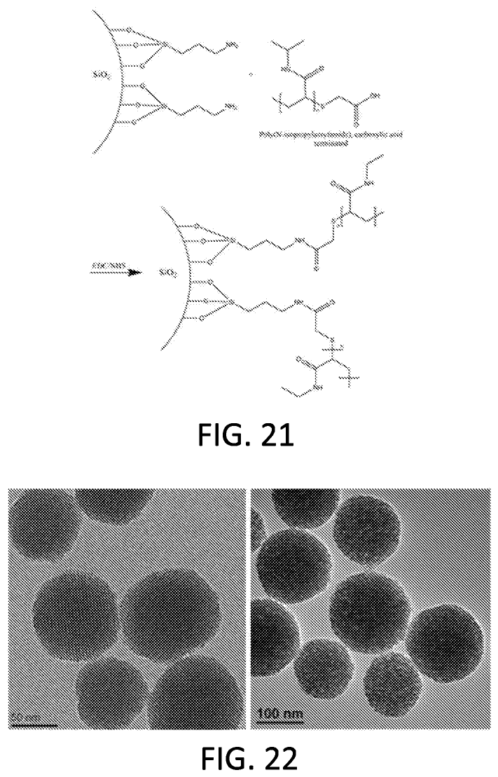

FIG. 21 is a schematic illustration of an example method for producing a PNIPAm capped MSN. In one example, PNIPAm is synthesized by washing 1-S4 with HEPES buffer (20 mL) and redisperse it in 20 mL HEPES buffer for further use. 30 mg of PNIPAm was dissolved in cold MES buffer and stirred for 15 min, then 8.25 mg of EDC.HCl and 9.3 mg of sulfo-NHS were added. The mixture was stirred for one hour in room temperature, then add to 5 mL HEPES buffer with 50 mg of dispersed S1-S4. The mixture was stirred for 24 h, and products were collected by centrifugation (15 min at 7830 rpm) at 20° C. followed by washing 8× with cold DI water (8×20 mL). S1-S4 were redispersed in 20 mL DI water for further use.

In another example, 160 mg of amine-modified MSNs is washed in DI water for 2 times (2×20 mL) and one time in 1×PBS buffer (20 mL). Redisperse it in 5 mL PBS buffer for further use. Dissolve 80 mg PNIPAm in cold MES buffer and stir for 15 min, then add 27.4 mg of EDC and 40.9 mg of NHS. Stir for one hour, then add dispersed MSNs. Keep stirring for 24 h, and collect product by centrifugation (15 min at 7830 rpm). Wash 8x with cold DI water and centrifuge at 20° C. Redisperse in 20 mL of DI water for further use.

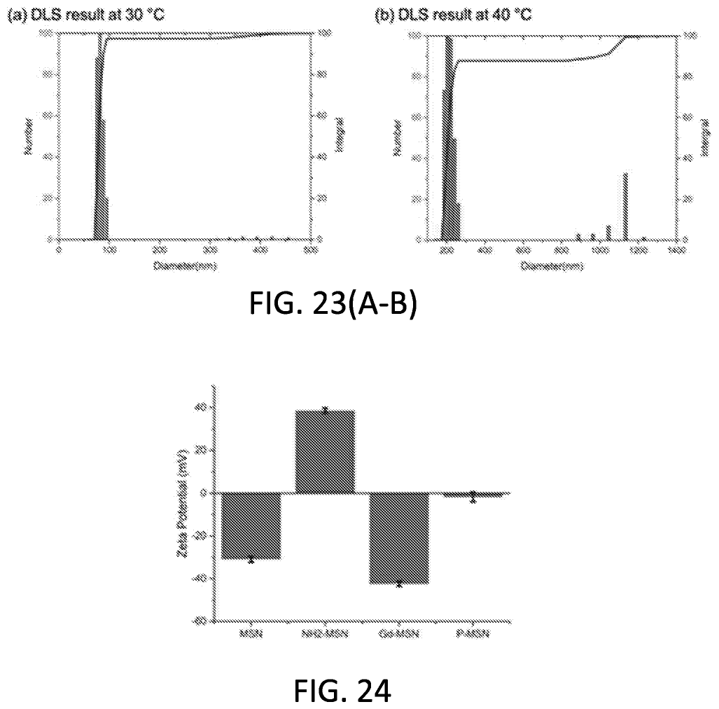

PNIPAm was attached to the amine group by EDC/NHS reaction, which is shown in FIG. 2. TEM images (FIG. 22) showed the PNIPAm-MSN stay intact but looked blur because of the PNIPAm. DLS results (FIG. 23) shows increasing diameter indicating aggregation due to the hydrophobicity of PNIPAm above LCST. Zeta-potential of MSN changed from negative to positive (amine modification), then to neutral (PNIPAm modification), as shown in FIG. 24. Weight loss could be observed from TGA results after each modification step.

### Tissue Mimicking Gel and MRI-Guided HIFU Sample Preparation

One gram of methyl cellulose was slowly added to 15 mL boiled water and stirred for 3 min. Then 25 mL condensed milk was added followed by 10 mL cold water. The mixture was stored in refrigerator overnight to eliminate air bubble. 3 mg of Gd-P-MSNs were dispersed in 0.5 mL water and then mixed with 1 mL gel/milk mixture, resulting a 2 mg/mL Gd-P-MSNs gel/milk mixture. The Magnevist (Mgv) control was made by similar method. Mgv was first diluted to 0.5 mL water, then was mixed with 1 mL gel/milk mixture.

### Agarose Phantom

17.5 g of agarose was slowly added to hot water with stirring. Then the solution was heated up to boiling, and then poured to sample holder model. After agarose was solidified under room temperature, it was stored in refrigerator for further use.

### Characterization

Transmission electron microscopy (TEM) images were recorded on a Tecnai T12 Quick CryoEM at an accelerating voltage of 120 kV. A suspension (8 μL) of nanoparticles in ethanol was dropped on a 200 mesh carbon coated copper grid and the solvent was allowed to evaporate at room temperature.

Zeta-potential analysis and DLS were carried out on a ZetaSizer Nano (Malvern Instruments Ltd., Worcestershire, U.K.) in DI water.

TGA was performed using a Perkin-Elmer Pyris Diamond TG/DTA under air (200 mL/min). Approximately 5-10 mg of sample was loaded into aluminum pans. The sample was held at 100° C. for 30 minutes, and then the data were recorded during a temperature scan from 100 to 600° C. at a scan rate of 10° C./min and an isothermal process of 600° C. for 80 min. The plotted values are normalized to the weight at 100° C. An empty aluminum pan was used as a reference.

ICP-OES measurements were made using ICPE-9000 Shimadzu. 0.1 mL of sodium hydroxide solution (2 M) was added to approximately 0.5-1 mg sample dispersed in 0.05 mL of Milli-Q water, and the mixture was sonicated for 1 h. Then 0.05 mL of nitric acid was added, and the mixture was sonicated for 1 h. The solution was then diluted to 10 mL with 2% nitric acid for measurement.

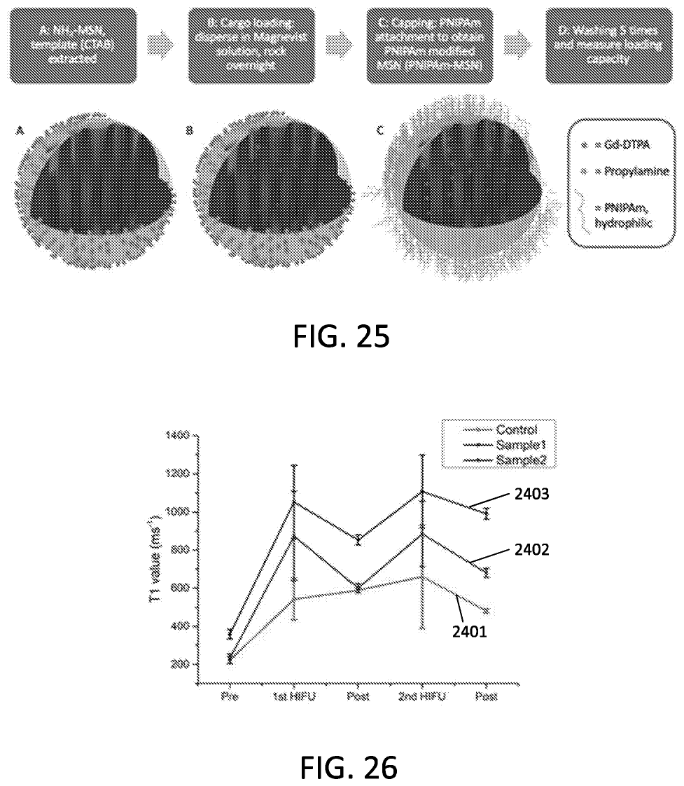

Referring to FIG. 25, an example synthetic route is illustrated for loading active agents into a PNIPAm capped MSN. As shown in step A, a co-condensation method for amine modification was tried but produced less than ideal coverage, so post-grafting method was adapted to synthesize NH₂-MSN. After MSN was synthesized, it was washed and refluxed in dry toluene with (3-Aminopropyl)triethoxysilane (APTES). The coverage of post-grafted APTES was also optimized according to delivery performance. After post-grafting, CTAB was extracted by refluxing in NH₄NO₃ ethanol solution. As shown in Step B, MSN was dispersed in Magnevist solution and rocked overnight. Loading solution with various cargo concentration was tested and turned out the highest concentration gave highest loading capacity. As shown in Step C, the capping sequence was also optimized based on loading capacity. Since PNIPAm is a reversible cap, cargo was loaded with PNIPAm attached first in high temperature, but the loading capacity was relatively low. But if Magnevist was loaded before PNIPAm attachment, the loading capacity will be much higher although during the attachment step the loading concentration was diluted.

### Active Agent Loading and Release:

The delivery performance experiments focus on uptake, loading and release capacity. The protocol is described as following:

Loading: 10-15 mg washed MSN was dispersed in 1-2 mL drug solution, stirred under desired temperature for certain amount of time. Then “capped” the delivery system by changing temperature, and trapped the active agent inside the MSN. Centrifuge MSN down and save the supernatant for concentration measurement. Uptake capacity was obtained from the formula below:

$$Uptake capacity = \frac{( {c}_{0} - {c}_{1} ) \times {V}_{0}}{{m}_{MSN}} \times 100 \%$$

where c₀ is the drug concentration before loading (mg/mL), c₁ is the drug concentration after loading (mg/mL), V₀ is the volume of active agent (e.g., drug) solution (mL), and m_{MSN} is the weight of the MSNs (mg).

Washing: dispersed MSNs in wash solution (e.g., DI water or PBS buffer). Then spun down and saved the supernatant for concentration measurement. Repeated 3˜4 times or until the drug concentration in last wash supernatant reached zero. Loading capacity, which showed the amount of drug actually trapped in MSN, was calculated as:

$$Loading capacity = uptake capacity - \frac{Σ {c}_{i} {V}_{i}}{{m}_{MSN}} \times 100 \%$$

where cᵢ is the drug concentration in the wash solution (mg/mL), and Vᵢ is the volume of the wash solution (mL).

Releasing: dispersed the washed MSNs in release solution (e.g., DI water or PBS buffer) and stirred for a duration. Then spun down MSNs and collected supernatant. One or two times of washes were usually applied after to ensure all released drug was collected. Release capacity was calculated as shown below:

$$Release capacity = \frac{{c}_{2} {V}_{2} + Σ {c}_{i} {V}_{i}}{{m}_{MSN}} \times 100 \%$$

where c₂ is the drug concentration after release (mg/mL), and V₂ is the volume of release solution (mL). The concentration of [Ru(bpy)₃]Cl₂ and Magnevist was measured quantitatively by UV-Vis or ICP-OES.

### Chemicals:

Tetraethyl orthosilicate (TEOS; 99%, Aldrich), cetyltrimethylammonium bromide (CTAB; 98%, Aldrich), sodium hydroxide (99%, Fisher Scientific), absolute ethanol (EtOH; Aldrich), Anhydrous toluene was obtained by distillation from CaH₂ under dry nitrogen.

Table 2 shows a sample experimental matrix to test which method produced the highest loading capacity of [Ru(bpy)₃]Cl₂.

TABLE 2
Sample matrix in delivery experiment

| | Sample name | Sample description |
|---|---|---|
| Hypothesis | RT loading, hot capping | Load in room temperature |
| one | (RLHC) | (RT), cap in hot water |
| | | bath, release in RT |
| | RT loading, no capping | In RT all the time, |
| | (RLNC) | control sample for capping |
| | RT blank (RB) | In RT all the time, control |
| | | sample for polymer |
| Hypothesis | Hot loading, RT capping | Load in hot water bath, |
| two | (HLRC) | cap by cooling down to |
| | | RT, release in hot water bath |
| | Hot loading, no capping | In hot water bath all the time, |
| | (HLNC) | control sample for |
| | | capping |
| | Hot blank (HB) | In hot water bath all the time, |
| | | control sample for |
| | | polymer |

According to the Table 2, the HLRC sample had the highest loading capacity. Also, higher temperature lead to “cap open” because hot cap causes more leakage during wash, and hot release leads to higher release percentage. Diffusion rate under different temperature can be calibrated by 2 samples without polymer, and from comparing the HB and RB samples, it can be observed that HB has lower uptake and release, which means high temperature did not lead to much higher loading and release capacity as expected. All samples with PNIPAm have similar uptake, and their uptake is higher than samples without PNIPAm, which means the PNIPAm did have an impact on delivery performance.

TABLE 3
Result of [Ru(bpy)₃]Cl₂ loading and release experiments of PNIPAm-MSN

| PNIPAm-MSN | HLRC | HLNC | HB | RLHC | RLNC | RB |
|---|---|---|---|---|---|---|
| Uptake | 2.85% | 2.73% | 1.59% | 2.66% | 2.85% | 1.85% |
| Wash | 1.34% | 2.01% | 0.60% | 2.33% | 1.59% | 1.47% |
| Loading | 1.51% | 0.72% | 0.99% | 0.33% | 1.26% | 0.38% |
| Release | 0.66% | 0.29% | 0.38% | 0.07% | 0.34% | 0.46% |
| (release %) | (43%) | (40%) | (38%) | 21%) | 27%) | (NA) |

Stöber SNP is spherical, non-porous silica nanoparticle with same chemical component on surface, which makes it easy to make the same surface modification as MSN. In this experiment, the diameter of Stöber SNP is 120 nm, which is the same as MSNs used. Same surface modification and characterization was applied on Stöber SNP, and the delivery experiment procedure and design was the same as well. In this way, every MSNs sample has a corresponding control sample with Stöber SNP.

TABLE 4
Result of [Ru(bpy)₃]Cl₂ loading and release experiments of Stöber SNP

| Stöber SNP | HLRC | HLNC | HB | RLHC | RLNC | RB |
|---|---|---|---|---|---|---|
| Uptake | 0.74% | 0.68% | 0.68% | 0.58% | 0.60% | 0.47% |
| Wash | 0.31% | 0.48% | 0.29% | 0.38% | 0.29% | 0.17% |
| Loading | 0.43% | 0.20% | 0.38% | 0.19% | 0.31% | 0.30% |
| Release | 0.04% | 0 | 0 | 0 | 0 | 0 |

The results of Stöber SNP as control group is shown in Table 4. The loading and release procedure were the same as previous experiment with MSN, but the loading and release was extremely low. It means the pores instead of PNIPAm polymer chain contribute to most of the loading capacity. Using the same loading and release condition above, Magnevist loading and release was tested. From the result shown in Table 4, “hot load, RT cap” had the highest loading capacity, which matched the previous results with [Ru(bpy)₃]Cl₂. Thus this condition was used in all following experiments for optimization.

TABLE 5
Result of Magnevist loading and release experiments of PNIPAm-MSN

| | | | Hot load, | | | RT load, |
|---|---|---|---|---|---|---|
| | Hot load, | Hot load, | no | RT load, | RT load, | no |
| | RT cap | hot cap | polymer | hot cap | RT cap | polymer |
| Uptake | 9.01% | 8.40% | 2.42% | 7.27% | 4.30% | 2.79% |
| Wash | 1.70% | 2.63% | 2.11% | 2.77% | 1.74% | 1.66% |
| Loading | 7.32% | 5.77% | 0.30% | 4.51% | 2.56% | 1.12% |
| Release | 0.055% | 0.044% | 0.017% | 0.037% | 0.065% | 0.026% |

### Optimization of Magnevist Loading Capacity of PNIPAm-MSN Delivery System:

Various optimization strategies were tried to improve the loading capacity. First, modifying PNIPAm before CTAB extraction was tested. In previous synthesis route, PNIPAm was attached after amine post-graft and CTAB extraction, which leave the pores unoccupied. So it is possible that PNIPAm is attached inside pore and hindering cargo diffusion. Therefore, PNIPAm modification was tried before CTAB extraction.

Table 5 shows the Magnevist loading performance. It can be noticed that the loading capacity dropped significantly, which may due to less PNIPAm coverage. It may because of the interference from the charge of CTAB during EDC/NHS and lower the yield. Thus, it was not used for further experiments.

TABLE 6
Comparison of proposed new method to old method

| | Hot load, RT cap | | RT load, RT cap | |
|---|---|---|---|---|
| | New method | Old method | New method | Old method |
| Uptake | 6.92% | 9.01% | 6.92% | 4.30% |
| Wash | 5.45% | 1.70% | 6.12% | 1.74% |
| Loading | 1.47% | 7.32% | 0.80% | 2.56% |

Second, wash with different solvents were tested. It is possible that if the unattached PNIPAm is not washed away sufficiently it could hinders the cargo diffusion, Both ethanol and water were used in the following experiment and from the loading capacity, their behavior were similar (Table 6). So water was used in all the following experiments.

TABLE 7
Comparison of two
proposed washing methods

| | Water wash | Ethanol wash |
|---|---|---|
| Uptake | 5.893% | 6.804% |
| Wash | 2.053% | 3.242% |
| Loading | 3.840% | 3.562% |

Third, various loading concentrations were tested. Concentration difference is the driving force of cargo diffusion, so loading solution with higher cargo concentration usually leads to higher loading capacity. Table 7 shows the results of using loading solution with 10 times and 100 times concentration as the one shown before. Loading capacity from 100 times loading concentration increased significantly, thus this concentration was used for the following two optimization methods.

Fourth, modifying pore wall with various amine groups was tested. Gd(DTPA)²⁻ has a negative charge in neutral pH, and amine group has a positive charge. Loading capacity may be improved by electrostatic attraction between Gd(DTPA)²⁻ and the amine group modified in pores. However, the loading capacity did not increase as expected, so such modification was not adapted in the following experiment.

Firth, loading Magnevist before PNIPAm modification was tested. After PNIPAm is grafted, even at opening state, there will still be spatial hindrance for cargo diffusion. Therefore, the cargo was tried loaded before PNIPAm was grafted. As shown in Table 8, the loading capacity was increased.

TABLE 8
Comparison of loading capacity from different loading concentrations

| | | 10 times | 100 times | | Loading |
|---|---|---|---|---|---|
| | Before | loading | loading | Amine | before |
| | opti- | concen- | concen- | modification | PNIPAm |
| | mization | tration | tration | in pore | grafting |
| Uptake | 9.01% | 24.11% | 71.50% | 56.99% | 78.24% |
| Wash | 1.70% | 13.08% | 23.10% | 30.39% | 23.28% |
| Loading | 7.32% | 11.03% | 48.41% | 26.61% | 54.96% |

As a summary, higher loading concentration and PNIPAm grafting after loading were two strategies that successfully increased Magnevist loading capacity. The optimized loading condition was used to prepare sample in the following MRI and HIFU experiments.

All MRI-guided HIFU experiments in Example 2 were conducted using a research-dedicated HIFU system (Image Guided Therapy, Bordeaux, France) integrated with a whole-body 3 T scanner (Prisma, Siemens Healthineers, Erlangen, Germany). The HIFU system had an 8-element annular transducer array with a diameter of 25 mm, frequency of 2.5 MHz, a focal point of 0.7×0.7×3 mm³ in size, and a peak electrical power output of 200 W. The electrical power output during experiments ranged from 18 W to 24.5 W.

T₁-weighted images were acquired before and after HIFU stimulation with a 3D Cartesian gradient-echo sequence using the following parameters: field of view (FOV)=280×140×54 mm³, matrix size=256×128×18, echo time (TE)=1.89 ms, repetition time (TR)=5 ms, flip angle=10°. T₁ relaxation times were measured before, during, and after HIFU stimulation using a Cartesian variable flip angle sequence with the following parameters: FOV=180×90×48 mm³, matrix size=192×96×16, TE=2.29 ms, TR=6 ms, flip angles=1, 2, 5, 7 and 9°. To correct B₁+ field variations, a separate B₁ mapping protocol was ran before, during, and after HIFU with matching FOV and matrix size with the T₁ mapping protocol and TE=1.87 ms, TR=2 s and flip angle=10°. These images were reconstructed in-line with the scanner software. A standard variable flip angle T₁ fitting algorithm was then carried out in an offline MATLAB 2018a (MathWorks, Natwick, Mass.) script to produce 3D T₁ maps. They were saved as DICOM images and imported into Horos where regions of interest (ROIs) of 9 voxels in size were carefully drawn to exclude the thermal probe and/or air bubbles inside the heated region of the sample to compute the average T₁ value.

### MRI-HIFU Experiments of Controlled Contrast Agent Release:

FIG. 26 shows the result of a HIFU experiment. MSNs were about 120 nm in diameter. The T₁ change caused by Gd-P-MSNs was then tested under MRI-guided HIFU.

MSN was dispersed in gel/milk and the mixture is placed in agarose phantom. The power and duration of HIFU had been adjusted to 18 W and 5 min, which ensured the temperature during HIFU was at least 34° C., so that it was higher than LCST of PNIPAm according to temperature mapping result. Control and both samples have the same amount of Magnevist and went through HIFU with the same power and duration. After the first HIFU, T₁ became significantly longer, which reflects the Magnevist release. After the second HIFU, T₁ showed a further increase. This indicates that the tuning of HIFU stimulation durations could be utilized to control the dose of released cargo. The control sample included Magnevist and water solution. Sample 1 included Magnevist loaded PNIPAm-MSN, batch 1. Sample 2 included Magnevist loaded PNIPAm-MSN, batch 2.

### Example 3: Exemplary Materials, Methods of Fabrication, and Characterization of Thermal-Responsive Silica Particles

### Material Design, Synthesis and Characterization

We designed Gd-DTPA and poly(N-isopropylacrylamide) (PNIPAm) modified MSNs (Gd-P-MSNs) to generate reversible MRI T₁ relaxivity changes based on the mechanism shown in FIG. 1. The T₁ relaxation time can be modulated using HIFU by changing the water access to Gd-DTPA. PNIPAm, a thermo-responsive polymer with a lower critical solution temperature (LCST) of 32° C. changes its hydrophilicity reversibly. It is hydrophilic below the LCST and hydrophobic above it. HIFU-induced periodic temperature changes across the LCST of PNIPAm can modulate the hydrophobicity of PNIPAm and the water access to Gd-DTPA and thus modulate T₁-weighted MRI contrast.

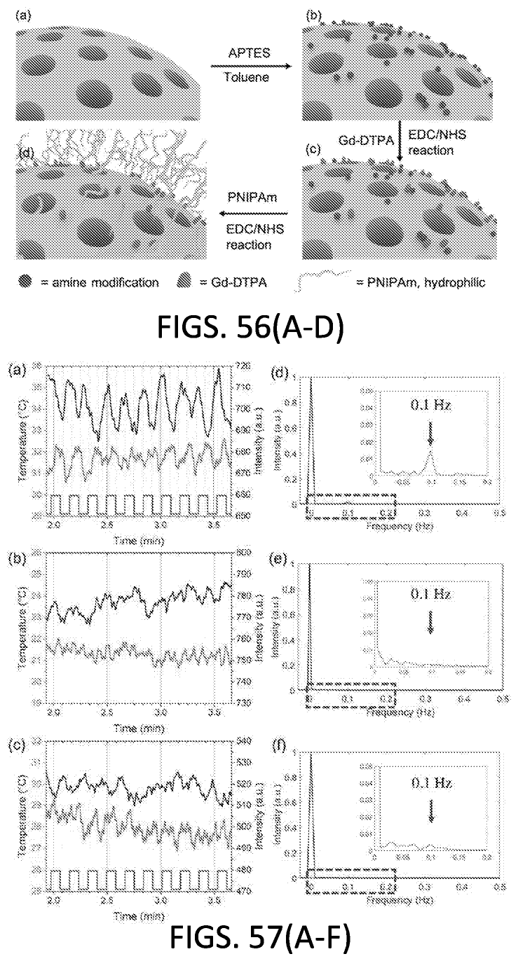

The Gd-P-MSNs was synthesized as shown in FIG. 56. MSNs were about 120 nm in diameter. They were modified by attaching amine groups mostly on the exterior surface by refluxing them in toluene with 3-aminopropyl triethoxysilane (APTES). The DLS and zeta potential were characterized using method mentioned in example 2. The amount of Gd-DTPA attached on MSNs was quantified by ICP-OES. PNIPAm with a carboxylic acid terminal was also attached to amine groups by the EDC/NHS reaction and quantified by TGA.

By tuning the amount of Gd(DTPA)²⁻ and PNIPAm, Gd-P-MSN of various Gd/PNIPAm mole ratios were synthesized. ICP-OES and TGA were used to quantify the attached Gd(DTPA)²⁻ and PNIPAm. Another modification strategy was also tried but did not work well, which is to modify amine group and Gd(DTPA)²⁻ on both the exterior and the interior (inside pore) surface of the particle. In this way, more Gd(DTPA)²⁻ was attached on the MSN because the interior surface area is greater than the exterior surface area. Moreover, Gd(DTPA)²⁻ is small enough to diffuse inside mesopores during the EDC/NHS reaction. PNIPAm was modified using the same method mentioned.

TABLE 9
Mole ratios of Gd(DTPA)²⁻ to PNIPAm

| | Gd(DTPA)²⁻ | PNIPAm | Mole ratio of |
|---|---|---|---|
| | weight | weight | Gd(DTPA)²⁻ |
| Sample name | % | % | to PNIPAm |
| Sample 1 | 0.44% | 12.01% | 0.47 |
| Sample 2 | 0.94% | 16.87% | 0.72 |
| Sample 3 | 0.65% | 9.88% | 0.84 |
| Sample 4 | 0.93% | 11.71% | 1.02 |
| Sample 5 | 1.79% | 10.33% | 2.25 |

The T₁ relaxivity change caused by Gd-P-MSNs was then tested under MRI-guided HIFU. Gd-P-MSNs was dispersed in Milli-Q water and mixed with a tissue-mimicking gel (methyl cellulose, 2 wt %) and milk (50% wt %). An agarose phantom (3.5 wt %) with cylindrical wells was constructed to hold samples. For simultaneous acquisition of the change in temperature and T₁-weighted image signal, a 3D multi-echo gradient-echo stack-of-radial sequence was used with FOV=109×109×30 mm³, matrix size=96×96×10, six echoes, TE1/ΔTE=1.43/1.29 ms, TR=11.1 ms, flip angle=6° and number of radial spokes=3000. Reconstruction was performed offline in MATLAB. To increase the temporal resolution, a k-space weighted image contrast (KWIC) filter was employed with eight annuli in total and the number of spokes in each annulus following the Fibonacci numbers 2, e.g., 3 (innermost annulus), 5, 8, 13, 21, 34, 55 and 87 (outermost annulus). The filter then moved 5 radial spokes at a time for a temporal resolution of 0.33 s and a temporal footprint of 9.88 s. Gridding, density compensation, and coil combination then followed to produce magnitude and phase images.

Magnitude images of all echoes were combined with sum-of-squares and a fast Fourier transform was performed on a voxel-by-voxel basis along the time dimension for spectral analysis and producing the modulation enhancement map (MEM). Phase images of all echoes were also combined to an effective TE=10 ms and relative temperature change was extracted from the phase difference between a dynamic image acquired at a time point t and the first dynamic image at baseline temperature before HIFU stimulation using

$$Δ T ( t ) = \frac{{ϕ}_{t} - {ϕ}_{0}}{α TE γ {B}_{0}} ,$$

where ΔT is the change in temperature at t, Φt is the phase at t, Φ0 is the phase of the first dynamic image, a is the proton resonant frequency shift (PRF) temperature coefficient of −0.01 ppm/° C., TE is the effective echo time (10 ms in this study), y is the gyromagnetic ratio of protons (267.522×106 rad s⁻¹·T⁻¹), and B₀ is the magnetic field strength.

Similar to the measurement of T₁ relaxation times, ROIs of 9 voxels in size were drawn to compute the average relative temperature change and magnitude change. The same ROIs were also transferred to MEMs to measure the intensity of the 0.1 Hz peak. For comparison, ROIs of 9 voxels and 100 voxels were drawn in unheated regions of the agar gel phantom and background noise, respectively, in the same images.

With a periodic temperature change across the LCST, PNIPAm can modulate the water access to Gd(DTPA)²⁻ accordingly thus modulate MRI contrast. The heat effect of HIFU is utilized to trigger reversible hydrophobicity change of PNIPAm. Periodic HIFU was used to generate periodic temperature change. MSN was dispersed in gel/milk and the mixture is placed in agarose phantom. The power and duration of HIFU had been adjusted to 18 W and 5 min, which ensured the temperature during HIFU was at least 34° C., so that it was higher than LCST of PNIPAm according to temperature mapping result. Similarly, the cooling time was also optimized to be 5 min, after which the sample temperature would be below 25° C. according to measurement from temperature probe. Additional points at 3 min during and post HIFU were also measured to help plot the T₁ change in this process. In order to double check the reversibility without the temperature effect, T₁ was also measured before all HIFU and 30 min after all HIFU when the sample temperature reach equilibrium with room temperature (20° C.). All measurements mentioned in this session follow this protocol.

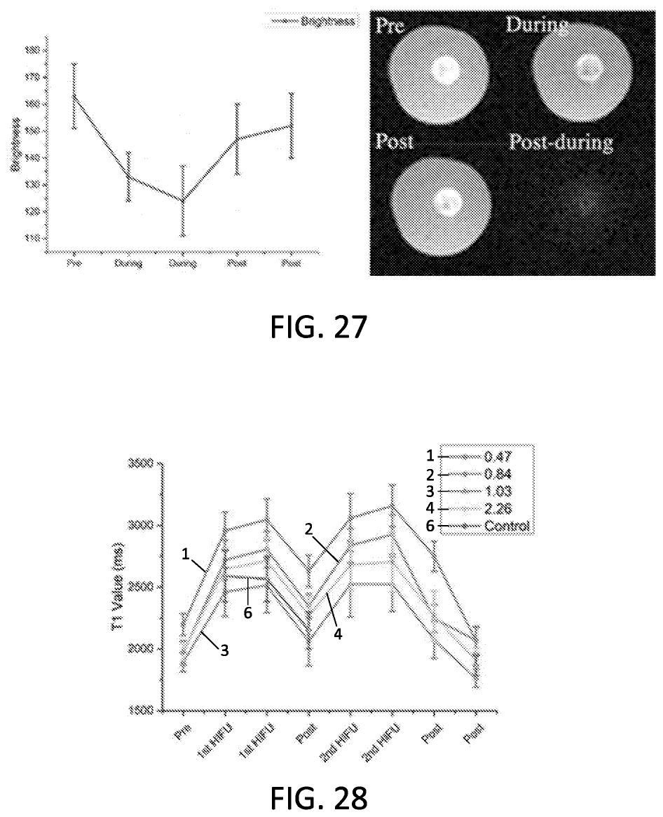

T₁-weighted and T₁ mapping images were obtained before, during and after HIFU. On T₁-weighted images, the brightness of Gd-P-MSN decreased during HIFU, and the brightness returned to the starting point after HIFU, indicating a reversible MRI contrast change caused by HIFU, as shown in FIG. 27. T₁ value was measured before, during and after HIFU for Gd-P-MSN with different Gd/P ratio and controls with only Magnevist or only Gd-MSN, as shown in FIG. 28. All of the samples and controls showed increasing T₁ value during HIFU and reversibility after two HIFU triggers, which is consistent with the previous T₁-weighted results. Samples 1-4 are each labeled 1-4, respectively in FIG. 28 and the control is labeled 6. In order to compare the T₁ increase of each sample and control, the T₁ value increase percentage (ΔT₁%) was calculated as:

$$Δ {T}_{1} \% = \frac{{T}_{1 , during} - {T}_{1 , post}}{{T}_{1 , post}} \times 100 \%$$

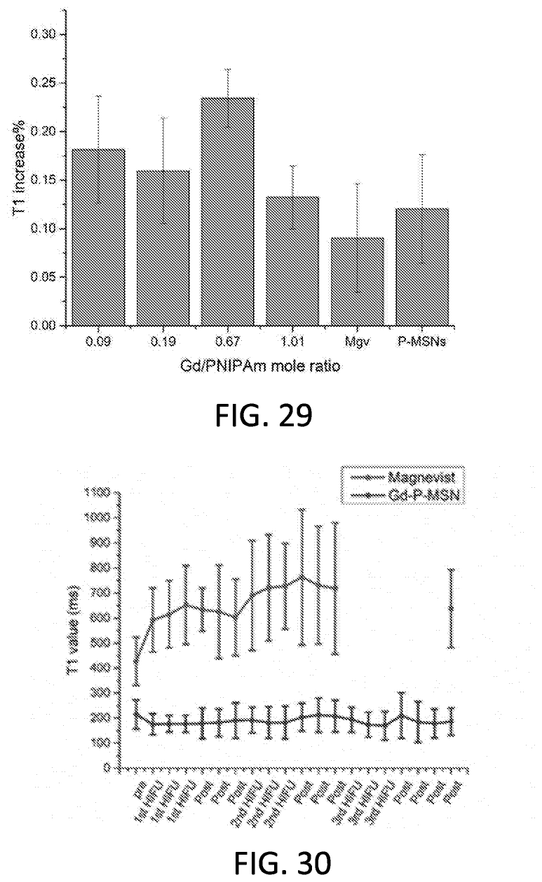

Referring to FIG. 29, it is shown that the value of ΔT₁% increased first and decreased as the ratio grew higher, which implies that the ΔT₁% is dependent on PNIPAm coverage. The samples around 0.67 showed higher ΔT₁% than Magnevist with the same amount of Gd(DTPA)²⁻, which indicated that they can cause more significant MRI contrast changes than Magnevist. Both control samples showed positive ΔT₁% due to temperature effect. Furthermore, Gd-MSN control showed lower ΔT₁% than all other samples with PNIPAm modification. This phenomenon further supported that the various ΔT₁% is relevant to PNIPAm modification instead of temperature effect alone. Referring to FIG. 30, The T₁ relaxivity was also measured for Gd-P-MSN that had Gd(DTPA)²⁻ modified both in pore and on outer surface. Neither T₁-weighted images nor T₁ mapping results showed significant change.

### Fast T₁ Relaxivity Modulation and Spectral Analysis

Both Gd-P-MSNs and Mgv were tested under periodic MRI-guided HIFU modulation. The HIFU power and repetition pattern were chosen to generate temperature modulation across the LCST within a short time. Gd-P-MSNs were pre-heated to 31° C. by HIFU using a power of 24.5 W for 3 min, followed by periodic HIFU stimulation with a power of 18 W to modulate the temperature across the LCST within a 2° C. window. Mgv was treated with the same HIFU sequence.

The frequency of the periodic HIFU modulation was 0.1 Hz with a 5 s on/off pattern, and the total duration was 100 s (10 cycles). Conventional Cartesian MRI protocols struggle to rapidly measure dynamic signal changes, therefore a 3D stack-of-radial golden-angle-ordered spoiled-gradient-echo multi-echo sequence (10 slices, TR=11.1 ms, six echoes, TE₁=1.43 ms, echo spacing=1.29 ms, flip angle=61) was used and coupled with a k-space weighted image contrast (KWIC) reconstruction technique (3 radial angles in the centermost annulus and 89 in total) to produce one set of TiW 3D images every 0.33 s. The temperature was simultaneously measured with this stack-of-radial sequence using the proton resonance frequency method, which was based on frequency and phase changes. The Gd-P-MSNs were also scanned with the same stack-of-radial sequence and duration without HIFU modulation as a control (Gd-P-MSNs no HIFU).

The HIFU sequence and T₁-weighted (T1 W) intensity changes of a Gd-P-MSN sample is shown in FIG. 11 A-B. There were no substantial changes in T1 W signal in the phantom or background area in all 3 experiments. T1 W signal changes due to HIFU modulation can be observed in both Gd-P-MSNs with HIFU and Mgv with HIFU, and the frequency of signal change follows that of the HIFU sequence. FIG. 57(a-c) shows the temperature and T1 W signal changes of the three samples during 100 seconds of periodic HIFU modulation. In both Gd-P-MSNs with HIFU and Mgv with HIFU, temperature and T1 W intensity changes were periodic and correlated: when the temperature increased, the T1 W intensity decreased, which is consistent with the results mentioned above. However, in Gd-P-MSNs no HIFU, the temperature and intensity changes were minor and random. In addition, the periodic T1 W signal changes in Gd-P-MSNs with HIFU were substantially larger than that in Mgv with HIFU.

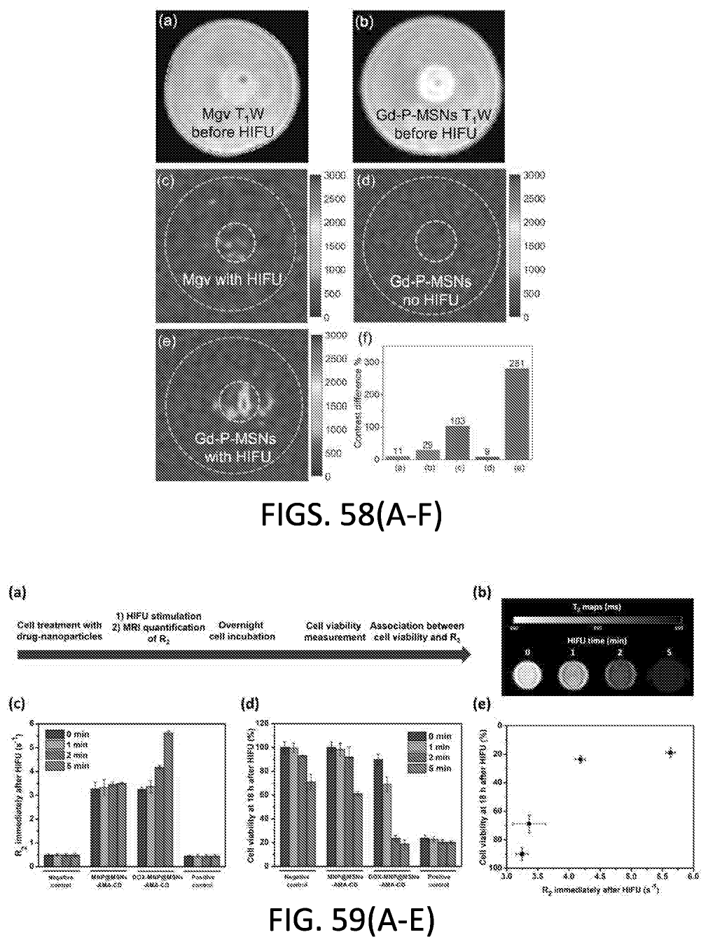

Spectral analysis was then done to construct a modulation enhancement map (MEM) using an offline MATLAB script. For each pixel, the change of its intensity in all dynamic T1 W images were plotted against time, after which a fast Fourier transform was performed to produce a frequency spectrum. FIG. 57 (d-f) show spectra from pixels in the HIFU focal points where the peak having the same frequency (0.1 Hz) as the HIFU modulation is indicated by the arrow. The intensity of this peak was normalized and then mapped across the entire imaging field of view to produce a MEM. The T1 W images before HIFU of Mgv (Mgv T1 W before HIFU) and Gd-P-MSNs (Gd-P-MSNs T1 W before HIPU) are shown in FIG. 58 (a-b). FIG. 58 (c-e) show the MEMs of Mgv with HIFU, Gd-P-MSNs no HIFU and Gd-P-MSNs with HIFU. FIG. 57(d-f) shows the frequency domain spectrum of one pixel in focal point.

To quantify the enhancement, contrast difference % (CD %) was calculated using the following formula, where μ_{A} stands for average intensity of region of interest (ROI), which is the sample region (within dotted inner circle in FIG. 58), μ_{B} stands for average intensity of the agarose phantom region (annulus between dotted inner circle and dotted outer circles in FIG. 58).

$$CD \% = \frac{{μ}_{A} - {μ}_{B}}{{μ}_{B}} \times 100 \%$$

The CD % achieved using the MSNs in combination with HIFU modulation is substantially higher than the 2 control cases (no MSNs with HIFU modulation; MSNs without HIFU modulation). In particular. The CD % of Gd-P-MSNs with HIFU is 281, which is 30-fold higher than CD % of 9 in Gd-P-MSNs no HIFU, close to 3-fold higher than CD % of 103 in Mgv with HIFU and 25-fold higher than CD % of 11 in Mgv T1 W before HIFU. From MEM of Gd-P-MSNs with HIFU, it is observed that the CD % at the 1.5 mm³ HIFU focal point is 912, which is 83-fold higher than CD % of Mgv T1 W before HIFU. This indicates that spectral analysis can efficiently capture the enhancement created by periodic HIFU modulation of Gd-P-MSNs. Also, Gd-P-MSNs clearly exhibited greater enhancement in the MEM than the Mgv undergoing the same periodic HIFU modulation, because it caused more T₁ change during HIFU as explained previously. The T1 W image before HIFU and MEM for Gd-P-MSNs both show several-fold higher CD % than Mgv, and these two could be considered together in a multi-spectral method to further enhance the contrast. MEMs were also constructed from the modulation in first 30 s, and Gd-P-MSNs with HIFU showed 46-fold enhancement compared to Mgv TIW before HIFU. It indicates that enhancement can also be achieved using a shorter data acquisition time.

Example 3 demonstrates HIFU-responsive Gd-P-MSNs that can generate reversible Tt changes by modulating the hydrophobicity of PNIPAm. Combined with periodic HIFU modulation at 0.1 Hz over 100 s and spectral analysis, the MRI contrast was enhanced by over an order of magnitude compared to that of Gd-P-MSNs without HIFU modulation, 3 times that of Mgv with HIFU modulation and 83 times that of Mgv with conventional Cartesian T1 W protocols. The method integrates these effects with the precise three-dimensional spatial control of the HIFU focal point to spotlight the region of interest with highly specific MRI contrast enhancement. The data acquisition time for the experiments in our study was only 100 s, and the small temperature change would cause minimal tissue damage. This method can be applied in improving the identification of target tissues, such as delineation of the tumor margins, for MRI-guided HIFU therapies.

### Example 4: Exemplary Materials, Methods of Fabrication, and Characterization of Mechano-Responsive Silica Particles

### Material Synthesis and Characterization

MSNs are synthesized by the method mentioned in example 2. The MSN surface modification route is shown in FIGS. 52-53. Gd-DTPA is coupled to amine-functionalized MSNs by EDC/NHS reaction. A poloxamer comprising poly(ethylene oxide) and poly(propylene oxide) (e.g., EO/PO/EO poloxamer or PO/EO/PO poloxamer) is coupled to 3-(triethoxysilyl)propyl isocyanate and condensed to Gd-DTPA modified MSNs.

A panel of poloxamer-Gd-MSNs were synthesized with different molecular weights and PO/EO ratios as listed in Table 10. Then dynamic light scattering (DLS), zeta-potential measurement and inductively coupled plasma-optical emission spectrometry (ICP-OES) was used to characterize the hydrodynamic diameters, surface charge and coupled Gd-DTPA on MSNs. As shown in Table 10, after poloxamer modification, the hydrodynamic diameters increased from 131 nm to 200-300 nm. The zeta-potential turned from positive to negative and the coupled Gd-DTPA weight percentage was 0.2-0.6%. The functionalized 25R2 polymer was also quantified by thermogravimetric analysis (TGA): the weight loss of Gd-DTPA modified MSNs was 14.85%, whereas after 25R2 coupling, the weight loss increased to 56.09%, which indicated that the weight percentage of coupled 25R2 polymer was 41.24%. 25R2, 17R4, 31R1, and P123 are commercially available poloxamers (Pluronic® provided by BASF Corporation).

TABLE 10
DLS, zeta-potential and ICP-OES
characterization of panel of poloxamer-Gd-
MSNs

| | Molecular | EO block | Hydro- | | Gd- |
|---|---|---|---|---|---|
| | weight of | weight % of | dynamic | Zeta- | DTPA |
| | poloxamer | poloxamer | diameters | potential | weight |
| | polymer | polymer | (nm) | (mV) | % |
| amine- | — | — | 124 | 10.95 | — |
| functionalized | | | | | |
| MSNs | | | | | |
| Gd-DTPA | — | — | 131 | 21.95 | 0.31% |
| modified MSNs | | | | | |
| 25R2-Gd-MSNs | 3600 | 20 | 245 | −43.07 | 0.27% |
| 17R4-Gd-MSNs | 2700 | 40 | 335 | −32.35 | 0.56% |
| 31R1-Gd-MSNs | 3300 | 10 | 267 | −27.79 | 0.46% |
| P123-Gd-MSNs | 5800 | 30 | 237 | −32.15 | 0.22% |

### MRI-Guided HIFU Experiments and Results

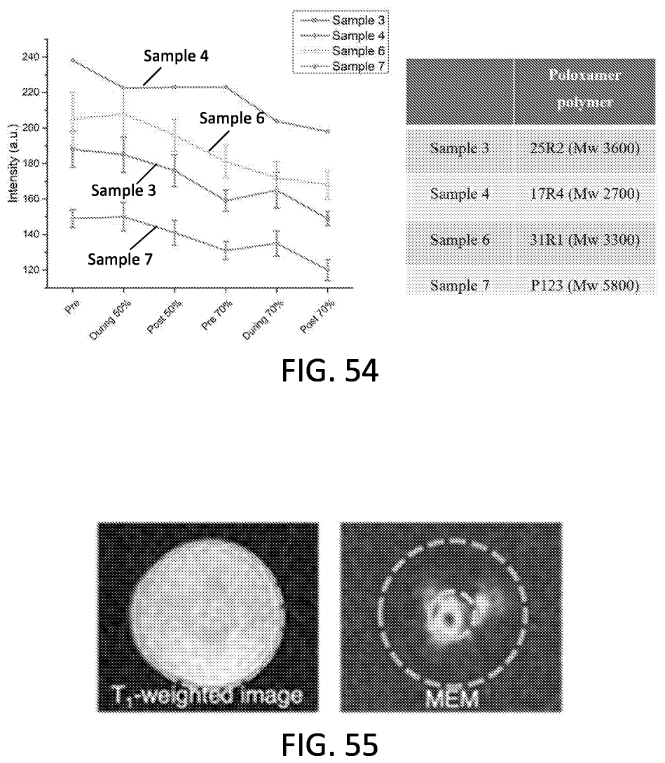

To evaluate the HIFU response of poloxamer-Gd-MSNs, a series of MRI-guided HIFU (MRI-HIFU) experiments were designed and conducted. First, MRI-HIFU experiments using a Cartesian T₁-weighted MRI protocol were designed to screen the poloxamer polymers from the panel and identify a suitable HIFU power for each polymer. According to the proposed T₁ modulation mechanism, the T₁ relaxation time will be shorter during HIFU, which leads to a higher intensity on T₁-weighted images. As shown in FIG. 54 both 25R2-Gd-MSNs and P123-Gd-MSNs showed higher T₁-weighted intensity when triggered with HIFU of 70% amplitude, and 31R1-Gd-MSNs showed higher T₁-weighted intensity when triggered with HIFU of 50% amplitude. However, 17R4-Gd-MSNs showed a trend that was the opposite of our expectation; this was mainly due to the thermal effect. Therefore, the panel except 17R4-Gd-MSNs was further investigated in the following experiments.

Next, MRI-HIFU fast modulation experiments were conducted using a dynamic 3D stack-of-radial T₁-weighted MRI protocol. The poloxamer-Gd-MSNs was dispersed in Milli-Q water and mixed with a tissue-mimicking gel (methyl cellulose, 2 wt %). An agarose phantom (3.5 wt %) with cylindrical wells was constructed to hold samples. The control was Magnevist (Mgv) with the same amount of Gd-DTPA as the poloxamer-Gd-MSN samples, and the HIFU power and duration were kept constant for all samples and controls. Various HIFU modulation sequences were tested as shown in Table 11. Spectral analysis was then done to construct a modulation enhancement map (MEM) using an offline MATLAB script. To quantify the enhancement, contrast difference (CD %) was calculated using the following formula, where μ_{A} stands for average intensity of HIFU focal point, which is within the sample region (within inner dotted circle in FIG. 55, μ_{B} stands for average intensity of the agarose phantom region (annulus between inner dotted circle and outer dotted circle circles in FIG. 55).

$$CD \% = \frac{{μ}_{A} - {μ}_{B}}{{μ}_{B}} \times 100 \%$$

The CD % of MEM and T₁-weighted images were calculated for various HIFU modulation sequences, and the results are listed in Table 11. 31R1-Gd-MSNs did not show HIFU responsive modulation, so the results are not listed here.

TABLE 11
CD % and enhancement fold of 25R2-Gd-MSNs (R1-R5) and P123-Gd-MSNs (P1-P5).

| | | | | | Enhancement | |
|---|---|---|---|---|---|---|
| | HIFU | HIFU | | | compare to | Enhancement |
| Trial | frequency | cycles and | HIFU | | T₁-weighted | compare to |
| number | (Hz) | duration | amplitude | CD % | image | Mgv control |
| R1 | 0.25 | 20, 80 s | 70% | 641% | 302.2 | 3.6 |
| R2 | 0.25 | 40, 160 s | 70% | 976% | 426.5 | 3.2 |
| R3 | 0.5 | 40, 80 s | 70% | 306% | 133.8 | 0.5 |
| R4 | 0.25 | 20, 80 s | 50% | 1332% | 254.7 | 92.5 |
| R5 | 0.5 | 40, 80 s | 50% | 793% | 161.7 | 118.2 |
| P1 | 0.25 | 20, 80 s | 70% | 291% | 40.8 | 1.6 |
| P2 | 0.25 | 40, 160 s | 70% | 626% | 2959.1 | 2.1 |
| P3 | 0.5 | 40, 80 s | 70% | 741% | 3504.8 | 1.2 |
| P4 | 0.25 | 20, 80 s | 50% | 661% | 387.5 | 45.9 |
| P5 | 0.5 | 40, 80 s | 50% | 481% | 129.9 | 71.8 |

For a decent enhancement, a high CD % is preferred, as well as high enhancement fold compare to T₁-weighted images and Mgv controls, A high CD % represent a high image contrast in MEM. The enhancement fold compare to T₁-weighted images is the ratio of CD % of MEM over that of T₁-weighted images of the same trial, which represents the enhancement achieved from the spectral analysis. The enhancement fold compare to Mgv control is the ratio of CD % of MSN sample's MEM over that of Mgv under the same HIFU modulation sequence, which shows the HIFU responsive contrast modulation from poloxamer-Gd-MSNs. From results in Table 11, it is observed that both 25R2-Gd-MSNs and P123-Gd-MSNs show significant contrast enhancement. FIG. 55 shows the T₁-weighted image and MEM of 25R2-Gd-MSNs, trial number R5. The T₁-weighted image does not show much contrast in the sample area, but the HIFU focal point is clearly shown in the MEM due to the 118-fold enhancement compared to Mgv control.

In this example, we have developed HIFU-responsive Pluronic-Gd-MSNs that can generate reversible T₁ relaxivity changes by modulating the water permeability of Pluronic polymer layer. We investigated a panel of nanoparticles modified with 4 different Pluronic polymers, and a series of HIFU sequences with different modulation frequency, cycle number and amplitude. With periodic HIFU modulation at 0.5 Hz over 80 s and spectral analysis, the MRI contrast was enhanced by over two orders of magnitude compared to that of images from conventional Cartesian T₁-weighted protocols and Mgv with HIFU modulation. Our new nanoparticle design responds to the mechanical effect of HIFU to utilize precise three-dimensional spatial control of the HIFU focal point to “spotlight” the region of interest with highly specific MRI contrast enhancement. In addition, this new nanoparticle expands the application of the spotlight technique with an alternative HIFU response mechanism.

### Example 5: Exemplary Materials, Methods of Fabrication, and Characterization of Silica Particles Having a Superparamagnetic Core(s) and Capping Agents Disposed on the Surface of the MSN

Synthesis of MnFe₂O₄ Nanoparticles:

MnFe₂O₄ nanoparticles were synthesized as follows. Two mmol of Fe(acac)₃, 1 mmol of Mₙ(acac)₂, 10 mmol of 1,2-dodecanediol, 6 mmol of oleic acid, and 6 mmol of oleylamine were dissolved in 20 mL of benzyl ether in a three-neck flask. The reaction was heated to 200° C. under the flow of nitrogen with vigorously stirring and kept at that temperature for 1 h. The reaction mixture was then heated up and refluxed for 1 h (298° C.). Afterwards, the resulting solution containing MnFe₂O₄ nanoparticles was cooled to room temperature. The nanoparticles were precipitated by adding 40 mL of ethanol and further separated by centrifugation (7830 rpm, 10 min).

MnFe₂O₄ nanoparticles were dispersed in 10 mL of hexane with 50 μL of oleic acid and 50 μL of oleylamine. The larger MnFe₂O₄ nanoparticles were synthesized by growing MnFe₂O₄ on the previously resulted MnFe₂O₄ nanoparticles through the similar procedure. Generally, two mmol of Fe(acac)₃, 1 mmol of Mₙ(acac)₂, 10 mmol of 1,2-dodecanediol, 2 mmol of oleic acid, and 2 mmol of oleylamine were dissolved in 20 mL of benzyl ether in a 100 mL three-neck flask. The MnFe₂O₄ nanoparticles (90 mg) obtained previously in 10 mL of hexane were added to the reaction mixture. The reaction mixture was heated to 90° C. and kept at that temperature for 30 min to remove hexane. Then the reaction mixture was heated to 200° C. under the flow of nitrogen with vigorously stirring. After 1 h, the reaction mixture was heated to 298° C. and refluxed for 1 h. The nanoparticles were precipitated by adding 40 mL of ethanol and further separated by centrifugation. The MnFe₂O₄ nanoparticles were re-dispersed in 10 mL of hexane with 50 μL of oleic acid and 50 μL of oleylamine. Synthesis of MnFe₂O₄@CoFe₂O₄ nanoparticles:

To coat CoFe₂O₄ on the surface of MnFe₂O₄ nanoparticles, Fe(acac)₃, Co(acac)₂, 1,2-dodecanediol, oleic acid, and oleylamine were dissolved in 20 mL of benzyl ether in a 100 mL three-neck flask. MnFe₂O₄ (8.2 nm) nanoparticles obtained previously in 10 mL of hexane were added to the reaction mixture. The synthetic procedure and reaction temperature were the same as that of the 8.2 nm MnFe₂O₄ nanoparticles synthesis. Finally, MnFe₂O₄@ CoFe₂O₄ nanoparticles (10.1 nm) were re-dispersed in 10 mL of hexane with oleic acid and oleylamine.

To coat CoFe₂O₄ on the surface of MnFe₂O₄ nanoparticles, two mmol of Fe(acac)₃, 1 mmol of Co(acac)₂, 10 mmol of 1,2-dodecanediol, 2 mmol of oleic acid, and 2 mmol of oleylamine were dissolved in 20 mL of benzyl ether in a 100 mL three-neck flask. The MnFe₂O₄ nanoparticles (180 mg) obtained previously in 10 mL of hexane were added to the reaction mixture. The synthetic procedure and reaction temperature were the same as that of the above-mentioned. Finally, MnFe₂O₄@CoFe₂O₄ nanoparticles were re-dispersed in 10 mL of hexane with 50 μL of oleic acid and 50 μL of oleylamine. To obtain larger MnFe₂O₄@CoFe₂O₄, another CoFe₂O₄ was further coated on the surface of the previously resulted MnFe₂O₄@CoFe₂O₄ nanoparticles. Two mmol of Fe(acac)₃, 1 mmol of Co(acac)₂, 10 mmol of 1,2-dodecanediol, 2 mmol of oleic acid, and 2 mmol of oleylamine were dissolved in 20 mL of benzyl ether in a 100 mL three-neck flask. MnFe₂O₄@CoFe₂O₄ nanoparticles (270 mg) obtained previously in 10 mL of hexane were added to the reaction mixture. The synthetic procedure and reaction temperature were the same as that of the above-mentioned. Finally, MnFe₂O₄@CoFe₂O₄ nanoparticles (11.0 nm) were re-dispersed in 10 mL of hexane with 50 μL of oleic acid and 50 μL of oleylamine for further use.

Synthesis of APTS-Functionalized MnFe₂O₄@CoFe₂O₄@Mesoporous Silica (Mag@MSNs-APTS) Core@Shell Nanoparticles

MnFe₂O₄@CoFe₂O₄ nanoparticles (11.0 nm, 2.5 mg) were dispersed in 0.2 mL of chloroform. 2 mL of CTAB aqueous solution (40 mg of CTAB, 54 mM) was added to the MnFe₂O₄@CoFe₂O₄ colloidal solution, and the mixture was sonicated for 10 min with a fully sealed cover to generate oil-in-water emulsion. The emulsion was then sonicated for 1 h to evaporate chloroform. The clear and well-dispersed MnFe₂O₄@CoFe₂O₄ colloidal aqueous solution (2 mL) was obtained (designated as MnFe₂O₄@CoFe₂O₄@CTAB). Meanwhile, 40 mg of CTAB was dissolved in 18 mL of water with 120 μL of NaOH solution (2 M) in a 100 mL flask.

The previously obtained MnFe₂O₄@CoFe₂O₄@CTAB colloidal solution (2 mL) was added to the reaction solution with vigorously stirring, and the temperature of the solution was brought up to 70° C. To coat mesoporous silica shell on the surface of MnFe₂O₄@CoFe₂O₄@CTAB, 200 μL of TEOS and 1.2 mL of ethyl acetate were added dropwise into the solution. After stirring for 2 h, 40 μL of APTS was added dropwise into the solution and stirred for another 2 h. The resulted amine functionalized MnFe₂O₄@CoFe₂O₄@MSNs was designated as MNP@MSNs-APTS (MNP denotes “magnetic nanoparticle”). Afterwards, the solution was cooled to room temperature and MNP@MSNs-APTS was centrifuged and washed 3 times with ethanol.

Subsequently, MNP@MSNs-APTS was dispersed in 20 mL of ethanol containing 120 mg of NH₄NO₃ and the reaction was stirred at 60° C. for 1 h to remove the surfactants. The surfactant removal procedures were repeated twice and MNP@MSNs-APTS was washed several times with deionized water and ethanol to obtain the surfactant-free MNP@MSNs-APTS

### Synthesis of ACVA Functionalized Mag@MSNs-APTS (Mag@MSNs-ACVA)

The conjugation of ACVA to the surface of MNP@MSNs-APTS was carried out by using an amide bond coupling reaction (Scheme S1). At first, the carboxylic acid of ACVA (20 mg) was activated by EDC (40 mg) and NHS (20 mg) in DMSO (4 mL). To crosslink the activated ACVA to the primary amine of APTS, after 30 min activation at room temperature, 20 mg of MNP@MSNs-APTS dispersed in DMSO (4 mL) were added dropwise to the activated ACVA in DMSO and stirred for 24 h. The ACVA functionalized MNP@MSNs-APTS (MNP@MSNs-ACVA) was washed, centrifuged, and re-suspended in DMSO three times to remove the excess ACVA, EDC, and NHS.

### Synthesis of AMA Functionalized Mag@MSNs-ACVA (Mag@MSNs-AMA)

The conjugation of AMA to the surface of MNP@MSNs-ACVA was carried out through amide bond formation between the carboxylic acid group of ACVA and the primary amine of AMA (Scheme S1). Typically, the carboxylic acid groups of MNP@MSNs-ACVA (20 mg) were activated by EDC (40 mg) and NHS (20 mg) in DMSO (4 mL). To crosslink ACVA to AMA, after 30 min activation at room temperature, 20 mg of AMA dissolved in DMSO (4 mL) was added to the activated MNP@MSNs-ACVA in DMSO and stirred for 24 h. Finally, AMA functionalized MNP@MSNs-ACVA (MNP@MSNs-AMA) was washed, centrifuged, and re-suspended in DMSO three times to remove the excess AMA, EDC, and NHS.

### Loading of DOX or Fluorescein in Mag@MSNs-AMA and Snap-Top Attachment (Mag@MSNs-AMA-Cyclodextrin)

The loading of doxorubicin (DOX) or fluorescein was carried out by using water as the solvent. In general, 1 mg of MNP@MSNs-AMA was dispersed in deionized water (1 mL) with 3 mM fluorescein or DOX. After stirring for 24 h, 16 mg of the β-CD capping agent was added to the solution to prevent DOX or fluorescein from being released. The sample was designated as DOX- or fluorescein-loaded MNP@MSNs-AMA-CD. After mixing for 48 h, the DOX- and fluorescein-loaded MNP@MSNs-AMA-CD were centrifuged. The DOX-loaded MNP@MSNs-AMA-CD was washed with water seven times followed by PBS twice, and the fluorescein-loaded MNP@MSNs-AMA-CD was washed with water five times, to remove the excess DOX or fluorescein molecules. The final product was suspended in PBS or deionized water for further stimulated cargo release experiments.

### Ultrasound-Stimulated Release of DOX or Fluorescein by a Probe Sonicator

DOX-loaded MNP@MSNs-AMA-CD solution (0.75 mg/mL, 1 mL of PBS) or fluorescein-loaded MNP@MSNs-AMA-CD solution (0.75 mg/mL, 1 mL of deionized water) was prepared in an Eppendorf tube. The tip of the probe sonicator (VCX 130, Sonics & Materials, Inc, Newtown, USA) was placed in the center of the solution. The ultrasound probe was set to a frequency of 20 kHz and output power of 21 W (power density: 75 W/cm²). After various time durations of the ultrasound stimulation, the solution was centrifuged. The supernatant and pellet were collected separately for further quantification of DOX loading capacity and release efficiency by the plate reader (Tecan M1000).

### MRI-Guided High-Intensity Focused Ultrasound (MRgHIFU)-Stimulated Release of DOX

All MRgHIFU experiments were conducted using a research HIFU system (Image Guided Therapy, Bordeaux, France) integrated with a whole-body 3 T MRI scanner (Prisma, Siemens Healthineers, Erlangen, Germany). The HIFU system had a 128-element annular transducer array with a diameter of 9 cm, frequency of 1 MHz, a focal point of 1×1×7 mm³ in size, and a peak electrical power output of 1200 W. The electrical power output used ranged from 9 W to 290 W. DOX-loaded MNP@MSNs-AMA-CD solutions (0.15 mg/mL, 3 mL of PBS) were placed in sample wells (1.3 cm×1.3 cm×5 cm) in the agarose phantom (10 cm×10 cm×11.5 cm). The agarose phantom was placed on top of the HIFU transducer, which was secured on the patient table of the 3 T MRI scanner.

Through both mechanical and electronic steering of the HIFU transducer, the focal point was placed at the center of the sample well. The samples were stimulated by HIFU at electrical power levels of 74 W (power density: 7400 W/cm²) or 9 W (power density: 900 W/cm²) for different durations (from 1 to 10 min). T₂ maps were acquired before and after the HIFU stimulation using a 2D turbo-spin-echo protocol (see the section T₂ mapping above) to compare the T₂ values. The subtracted T₂ maps were obtained by subtracting post-HIFU stimulation T₂ maps from pre-HIFU stimulation T₂ maps. The temperature of the solution during the HIFU stimulation was measured by a 2D single-slice gradient-echo MRI temperature mapping sequence with an image update rate of 1.8 seconds. To quantify the released amount of DOX, the HIFU-stimulated samples were removed from the phantom and spun down to separate the pellet and supernatant for fluorescence intensity measurement by the plate reader.

### Loading Capacity Analysis of DOX

After being loaded with 3 mM DOX and capped with β-CD, DOX-loaded MNP@MSNs-AMA-CD was washed thoroughly with water seven times followed by PBS twice to remove the excess DOX. The DOX-loaded MNP@MSNs-AMA-CD solution (0.75 mg/mL, 1 mL of PBS) was put in a hot water bath at 80° C. for 30 min to completely release the loaded DOX in the nanoparticles. The released DOX was separated from MNP@MSNs-AMA-CD by centrifugation and recorded by the plate reader. The fluorescence intensity of the released DOX was integrated from 585 to 595 nm. As nearly 100% of the loaded DOX was released through this bulk heating treatment (80° C. for 30 min), the recorded fluorescence intensities indicated the total amount of loaded DOX in MNP@MSNs-AMA-CD. The loading capacity of DOX was then calculated following the definition of loading capacity. (mass of DOX loaded in pores/mass of nanoparticles)×100%.

Release Efficiency of DOX after Ultrasound or HIFU Stimulation

DOX-loaded MNP@MSNs-AMA-CD stimulated by ultrasound or HIFU was centrifuged to separate the pellet and the supernatant. The collected supernatants containing the released DOX were then analyzed by the plate reader. The fluorescence intensity of the released DOX was integrated from 585 to 595 nm. The fluorescence intensity corresponding to the DOX released after being heated at 80° C. for 30 min was designated as 100% release. The release efficiency of DOX was then calculated following the definition of release efficiency: (mass of released DOX/mass of DOX loaded in pores)×100%.

ζ-Potential Value Measurement of MNP@MSNs-ACVA after Heating or HIFU Stimulation

0.75 mg of MNP@MSNs-ACVA was dispersed in 1 mL of water in an Eppendorf tube. For bulk heating treatment, the Eppendorf tubes containing the samples were put in a 37 or 80° C. hot water bath for 10 or 30 min, respectively. Afterwards, the solution was centrifuged and the nanoparticles were washed and redispersed in deionized water. For HIFU stimulation, the samples were stimulated with HIFU at a power of 74 W and a frequency of 1 MHz for 1, 5, or 10 min. Similarly, after the treatment, the solution was centrifuged and the nanoparticles were washed and redispersed in deionized water. Finally, the ζ-potential values of the samples after treatment were then measured,

### In Vitro Cytotoxicity

The viability of PANC-1 cells after the treatment of MNP@MSNs-AMA-CD or DOX-loaded MNP@MSNs-AMA-CD were examined by using a cell counting kit-8 (CCK-8) assay. The cells were seeded in 96-well plates at a density of 5×10³ cells per well in 200 μL of DMEM supplemented with 10% FBS and 1% antibiotics in a humidity-controlled incubator at 37° C. for 24 h attachment. After the attachment, the medium was removed and the cells were treated with 0, 10, 25, 50, 75, 100, 200, and 300 μg/mL MNP@MSNs-AMA-CD for 4, 24, 48, or 72 h, or 0, 10, 25, 50, 75, 100, and 200 μg/mL DOX-loaded MNP@MSNs-AMA-CD for 4 h in 200 μL of fresh DMEM in an incubator at 37° C.

After incubation, the medium was removed and the nanoparticle-treated cells were washed twice with DPBS. To measure the cell viability, DMEM (100 μL) and CCK-8 cellular cytotoxicity reagent (10 μL) were added to the cells in each well and incubated for 2 h at 37° C. The number of viable cells was determined by using the plate reader (Tecan M1000) to measure the absorbance at 450 nm and 650 nm (as the reference). DMEM (100 μL) containing the CCK-8 reagent (10 μL) served as a background. For the cell proliferation study, after 4 h treatment of 0, 10, 25, 50, 75, 100, and 200 μg/mL DOX-loaded MNP@MSNs-AMA-CD, the medium was removed and the nanoparticle-treated cells were washed twice with DPBS. The cells were allowed to grow in a fresh culture medium for another 18 h and the cell viability was determined by the CCK-8 assay as above.

### In Vitro MRgHIFU-Stimulated DOX Release and Cellular T₂ Monitoring

PANC-1 cells were seeded in 24-well plates at a density of 10′ cells per well in 500 μL of DMEM supplemented with 10% FBS and 1% antibiotics in a humidity-controlled incubator at 37° C. for 24 h attachment. After the attachment, the medium was removed and the cells were treated with DOX-loaded MNP@MSNs-AMA-CD (200 μg/mL) in 300 μL of fresh DMEM in an incubator at 37° C. The control groups including cells only (negative control), cells treated with an equivalent amount of free DOX to the DOX-loaded MNP@MSNs-AMA-CD (positive control), and cells treated with MNP@MSNs-AMA-CD (200 μg/mL) were also investigated. After 4 h incubation, the medium was removed and the cells were washed twice with DPBS (500 μL×2).

The cells were harvested by trypsinization with 0.05% trypsin-EDTA and suspended in DMEM. The cell suspensions were then transferred into 15 mL Falcon plastic tubes in preparation for MRgHIFU experiments. The research HIFU system (Image Guided Therapy, Bordeaux, France) integrated with the whole-body 3 T MRI scanner (Prisma, Siemens Healthineers, Erlangen, Germany) was used. To stimulate the release of DOX in cells by MRgHIFU, the cell suspensions were transferred into sample wells (1.3 cm×1.3 cm×5 cm) in the agarose phantom (10 cm×10 cm×11.5 cm). The agarose phantom was placed on top of the HIFU transducer, which was secured on the patient table of the 3 T MRI scanner. The HIFU focal point was placed at the center of the sample well and the cells were stimulated by HIFU at an electrical power level of 9 W (power density: 900 W/cm²) for different durations (0, 1, 2, or 5 min). MRI T₂ mapping was performed before and after the HIFU stimulation using a 2D TSE protocol (see the section T₂ mapping above) to compare the T₂ values.

The temperature of the sample during the HIFU stimulation was measured by a 2D single-slice gradient-echo MRI temperature mapping sequence with an image update rate of 1.8 seconds. After the HIFU stimulation and T₂ mapping, the treated cells were allowed to grow and attach in 96-well plates in 200 μL of DMEM supplemented with 10% FBS and 1% antibiotics in a humidity-controlled incubator at 37° C. for 18 h. The cell viability after the HIFU stimulation was measured by the CCK-8 assay. Basically, after removing the medium, DMEM (100 μL) and CCK-8 reagent (10 μL) were added to the cells in each well and incubated for 2 h at 37° C. The number of viable cells was determined by using the plate reader (Tecan M1000) to measure the absorbance at 450 nm and 650 nm (as the reference). DMEM (100 μL) containing the CCK-8 reagent (10 μL) served as a background.

Fluorescence Microscope Images of PANC-1 Cells after HIFU Stimulations

After the HIFU stimulation and T₂ mapping, the PANC-1 cells treated with DOX-loaded MNP@MSNs-AMA-CD (200 μg/mL) were allowed to grow and attach in 8-well chamber slides at a density of 2.5×10⁴ cells per well in 500 μL of DMEM supplemented with 10% FBS and 1% antibiotics in a humidity-controlled incubator at 37° C. After 18 h attachment, the cells were washed with DPBS three times (500 μL×3) followed by fixing with 4% paraformaldehyde in PBS for 20 min. The fixed cells were then washed with DPBS three times (500 L×3). Afterwards, the cell nuclei were stained with Hoechst 33342 (500 μL, 5 μg/mL) for 20 min followed by washing with DPBS five times (500 μL×5). The stained cells were covered by mounting medium cover glass before taking fluorescence images using a Zeiss fluorescence microscope.

### T₂ Mapping

T₂ maps of water-suspended DOX-loaded MNP@MSNs-AMA-CD before and after the stimulation by a probe sonicator or MRgHIFU were acquired using a 3 T MRI scanner (Prisma, Siemens Healthineers, Erlangen, Germany) with a 2D turbo-spin-echo (TSE) protocol. 1 mL of samples with and without HIFU stimulation were mixed with 3 mL of methylcellulose (2.5 wt %) in 15 mL Falcon plastic tubes placed in a water bath.

Parameters for the T₂ mapping protocol were: 2D multiple-TE TSE sequence; FOV=350×350 mm³; matrix size=256×256; slice thickness=3 mm; 20 slices; TEs=12, 24, 35, 47, 59, 83, 94, 118 ms; TR=8 s; excitation pulse flip angle=90°; refocusing pulse flip angle=180°. T₂ (ms) was calculated using a mono-exponential fitting algorithm.

T₂ Relaxivity (r₂) Measurement

Different concentrations of fluorescein- or DOX-loaded MNP@MSNs-AMA-CD, or ultrasound or HIFU-stimulated fluorescein-loaded MNP@MSNs-AMA-CD were mixed with 2.5 wt % methylcellulose. T₂ relaxation times were acquired by the 3 T MRI scanner using the above multiple-TE TSE sequence. r₂ (s⁻¹mM⁻¹) was calculated as the ratio of 1/T₂ to the total concentrations of Fe, Mn, and Co as determined by ICP-OES.

### Results/Discussion

In this Example, a stable drug carrier with both HIFU responsiveness and MR imageability is illustrated, where the drug carrier comprises a core-shell structure composed of a super-paramagnetic nanoparticle core and a mesoporous silica shell. The mesoporous silica offers a rigid structure with high surface area and large pore volume for high loads of cargo delivery and a precise control of the particle diameter. Other desirable properties such as good biocompatibility, high cellular internalization efficiency, and easy surface functionalization render the mesoporous silica nanoparticle a HIFU-responsive drug carrier.

The HIFU-responsive cap in this Example is designed to regulate the release of a clinically used model chemotherapeutic agent, DOX, from the nanoparticles in response to HIFU stimulation. With this nanosystem, T₂ changes in conjunction with drug release and thus the process of drug release can be self reported from the nanosystem via MRI, without the need for further computation to model the difference in physicochemical properties between drugs and contrast agents. Another application of this strategy is to predict the therapeutic efficacy of drug delivery in cancer cells from the associated T₂ changes seen immediately after HIFU stimulations, without the need to wait for a certain time period for the cell death to be measured.

The HIFU-responsive cap comprises an aliphatic azo-containing compound, 4,4′-azobis(4-cyanovaleric acid) (ACVA), which was attached on the surface of core-shell nano-particles composed of a manganese and cobalt-doped iron oxide magnetic nanoparticle (MnFe2O4@CoFe2O4) core and a mesoporous silica shell as described herein.

Covalent C—N bonds of ACVA are irreversibly cleavable by both ultrasound and heat, generating lower molecular weight fragments and nitrogen, thereby opening the pores and releasing DOX in response to HIFU stimulation. MnFe2O4@CoFe2O4 superparamagnetic nano-particles enhance T₂ effects due to its larger magnetization as compared to undoped superparamagnetic iron oxide nanoparticles.

Nanoparticles (MNP) were synthesized by a seed-mediated thermal decomposition process. The particles may have uniform size distribution (e.g., 11.0 nm) as observed by transmission electron microscopy (TEM). The surface of the mesoporous silica shell of the core-shell nanoparticles (MNP@MSNs) is first functionalized with amine groups from 3-(aminopropyl)triethoxysilane (APTS) (MNP@MSNs-APTS), and then grafted by amide bond-coupling reactions with ACVA and 1-adamantylamine (AMA) (MNP@MSNs-ACVA and MNP@MSNs-AMA, respectively).

The TEM images of MNP@MSNs-APTS and MNP@MSNs-AMA showed that the nanoparticles have similar morphology and mesoporous structures with average diameters of 54.4 nm and 55.2 nm, respectively, indicating that the nanostructure was not damaged after surface modification. Dynamic light scattering (DLS) analysis revealed that MNP@MSNs-APTS were well dispersed in water with an effective hydrodynamic diameter of 101.4 nm. The successful modifications of APTS, ACVA, and AMA were further confirmed by ζ-potential measurements, thermogravimetric analysis (TGA) (e.g., APTS is present in an amount of about 3% (w/w) based on the total weight of the nanoparticles, ACVA is present in an amount of about 3% (w/w) based on the total weight of the nanoparticles, and AMA is present in an amount of about 2% (w/w) based on the total weight of the nanoparticles), and Fourier-transform infrared spectroscopy (FT-IR) after each step.

The ACVA gatekeeper was stable on the nanoparticle's surface at physiological temperature (37° C.) as revealed by the similar negative ζ-potential value compared to that at room temperature (24° C.). On the other hand, the cleavage of ACVA occurred when the nanoparticles were heated at 80° C. for 30 min, leaving fewer carboxylic acid end groups on the nanoparticle's surface, and thus a neutral or positive charge was observed. After DOX loading, the nanoparticles were capped with bulky β-cyclodextrin (β-CD) (DOX-MNP@MSNs-AMA-CD) which is able to bind with the adamantane and form a supramolecular host-guest complex that covers the pores to prevent DOX leakage. The particles with the bulky hydrophilic β-CD caps retained the good water dispersibility of MNP@MSNs-AMA-CD and had an increased hydrodynamic diameter of 129.2 nm. The loading capacity of DOX in MNP@MSNs-AMA-CD, determined by fluorescence spectroscopy, was quantified as 4% of the original DOX (3 mM) loaded into 0.75 mg/mL of MNP@MSNs-AMA-CD. Successful capping was confirmed by negligible DOX leakage at both 24° C. and 37° C., again proving the applicability of this drug carrier at physiological temperature.

Ultrasound-responsiveness of the cap was first tested using a probe sonicator. The resulting DOX release from the uncapped pores was determined by fluorescence spectroscopy and quantified as DOX release efficiency, defined as (mass of released DOX/mass of DOX loaded in pores)×100%. DOX release efficiency increased as the ultrasound trigger time increased. The release rate exhibited two phases including an initial fast release and ensuing slower release. The temperature of the solution after 45 min of probe ultrasonication increased to 55° C. from room temperature of 24° C. Interestingly, the amount of DOX released stimulated solely by heating at 55° C. was lower than that released by ultrasound, implying that the mechanical effects of ultrasound (e.g., cavitation) play a role in inducing more ACVA cleavage and/or greater DOX diffusion.

The successful demonstration of the ultrasound-responsive cap removal and drug release prompted us to investigate whether the cargo release affects the MRI contrast in the presence of the na-noparticles. We conducted experiments using a research MRgHIFU platform, which has a HIFU system (1 MHz, peak electrical power output of 1200 W) integrated with a whole body 3 T MRI scanner. MRI and ultrasound research was conducted using an agarose phantom as the sample holder to mimic aqueous tissues. The agarose phantom was placed on the HIFU transducer on the patient bed in the MRI scanner.

Nanoparticles were placed in an aqueous suspension in a sample well in the agarose phantom and the whole phantom was moved into the MRI scanner for subsequent HIFU stimulation and MRI acquisition. Fluorescein was used as the cargo molecule and the fluorescein-loaded MNP@MSNs-AMA-CD were imaged via MRI before and after probe sonication or HIFU stimulation. We observed a significant increase (˜1.7-fold) in the spin-spin relaxivity (r2) in both cases. Another finding was that r2 increased as the amount of DOX loaded in the pores of nanoparticles decreased. The r2 value of the unloaded nanoparticles (415.9 s⁻¹mM⁻¹) was ˜1.7-fold greater than that of the DOX (20.6 μM)-loaded nanoparticles (242.1 s⁻¹mM⁻¹). The increase in r2 is primarily attributed to the cargo release, leaving more space in the pores that favors the access of water molecules to the MNP core, resulting in the enhanced T2 relaxation effect on water molecules in the vicinity of the MNP core.

To determine whether the change in MR signal can be used to monitor and characterize the amount of released drug, DOX-MNP@MSNs-AMA-CD was stimulated with various durations of HIFU stimulation (74 W) to vary the amounts of released DOX. R₂ (1/T₂) was quantified immediately after HIFU stimulation, and the released amounts of DOX were analyzed at several time points after HIFU stimulation over a period of up to 27 h. As expected, longer HIFU exposure times stimulated more DOX re-lease from the nanoparticle's pores, and the release exhibited the familiar biphasic profile with an initial accelerated release shortly after the HIFU stimulation followed by a slower release over time. The gradual increase of the nanoparticle's charge after HIFU stimulation corroborated the HIFU-induced ACVA cleavage. TEM images showed that the morphology and pore structure of the HIFU-stimulated nanoparticles remained intact. The nanostructure can withstand HIFU energy and the DOX release was mediated by the cap removal rather than the destruction of the nanoparticle structure. The bulk temperature increased to 24, 30, and 36° C. after 1, 5, and 10 min of HIFU stimulations, respectively, compared to room temperature of 24° C.

As the amount of DOX release increased with the longer HIFU stimulation time, R₂ increased correspondingly. The relative percentage change in R₂ may be defined as [100%×(post-R₂ pre-R₂)/pre-R₂], as a function of HIFU stimulation times. The changes in the MR signal could be visualized from their T₂ maps and subtracted T₂ maps (e.g., defined as subtracting post- from pre-release T₂ maps). DOX release efficiencies measured at 1.6 and 27 h after HIFU stimulation showed associations with R₂, indicating that the release of DOX can be characterized by using this DOX release versus R₂ plot. For example, from the observed change in R2 value from 6.7 s⁻¹ before HIFU-stimulated release to 16 s⁻¹ after release, we may predict that there will be around 32% and 94% of released DOX at 1.6 and 27 h after HIFU stimulation, respectively.

Based on the positive results of drug release characterization via MRI in bulk solution, we performed proof of concept in vitro experiments to investigate whether controllable drug release using our system can achieve therapeutic efficacy in cancer cells and whether that efficacy can be characterized via MRI (i.e. associated with R₂). Considering the delicacy of cells, HIFU with a lower power level (9 W) and shorter exposure times were employed.

The capability of these conservative HIFU parameters to stimulate DOX release and change R₂ was first confirmed by the familiar biphasic DOX release profile, R₂ as a function of HIFU stimulation times, and the DOX release versus R₂ plot in a bulk solution. No increase in bulk temperature was observed after 1 and 2 min of HIFU stimulations, with only 2° C. increase after 5 min of HIFU stimulation, from room temperature of 24° C. Human pancreatic cancer cells, PANC-1, were used for the in vitro studies. MNP@MSNs-AMA-CD have no intrinsic cytotoxi-city to PANC-1 as analyzed by a CCK-8 assay. After loaded with DOX, DOX-MNP@MSNs-AMA-CD showed minimal cytotoxicity to PANC-1 after 4 h of incubation suggesting minimal DOX leakage in the biological environment at 37° C. The proliferation of PANC-1 was unaffected by the negligible amount of leaked DOX during the 18 h growth period. Before MRgHIFU stimulation, cellular uptake of DOX-MNP@MSNs-AMA-CD was evidenced by the intracellular red fluorescence of DOX observed from fluorescence microscope images.

Referring to FIG. 59, an example workflow for characterizing the HIFU-stimulated drug delivery and its therapeutic efficacy in cells via MRI included cell treatment with drug-nanoparticles followed by (1) HIFU stimulation and (2) MRI quantification of R₂. The cells were incubated overnight. The cell viability was measured and the association between cell viability and R₂ was determined.

After treatment with DOX-MNP@MSNs-AMA-CD, the cells were stimulated with HIFU followed by imaging via MRI to quantify R₂. As expected, darker T₂ maps (FIG. 59B) and an increase in R₂ (FIG. 59C) from the cells as HIFU stimulation time increased were observed. Cell viability was measured after 18 h incubation to ensure sufficient DOX diffusion based on the time-dependent DOX release profile. The successful intracellular DOX release after HIFU stimulation was supported by the significantly decreased viabilities of DOX-MNP@MSNs-AMA-CD treated cells compared with those of the untreated cells (negative control) and the cells treated with un-loaded nanoparticles.

The cells treated with DOX-MNP@MSNs-AMA-CD but without HIFU stimulation had preserved viability showing that the HIFU-responsive cap is stable in the biological environment with negligible DOX leakage. Indeed, from the fluorescence images, the intracellular distribution of DOX varied before and after the HIFU stimulation, with more DOX deploying toward the nuclei suggesting the release of DOX, thereby inducing cell death. Decreased cell viability was observed as HIFU exposure time was increased (FIG. 59D), which may be a combinatory result from the drug therapeutic effect and the HIFU effects (thermal or mechanical). The drug effect dominated the HIFU effects, which was determined by comparing viabilities of cells treated with DOX-MNP@MSNs-AMA-CD, MNP@MSNs-AMA-CD, free DOX (positive control), and cells only (negative control) (FIG. 59D).

Two min of HIFU stimulation time released most of the loaded DOX after the 18 h incubation, as indicated by the comparable cell viability to that treated with an equivalent amount of free DOX, while this HIFU duration had minimal impact on cell viability in the cells treated with MNP@MSNs-AMA-CD and other controls (FIG. 59D). Comparing R₂ (FIG. 59C) with the viability of the cells treated with DOX-MNP@MSNs-AMA-CD showed an association (FIG. 59E), where the plot of loss of viability versus R₂ mirrored the plot of DOX release. This finding shows that the therapeutic efficacy of HIFU-stimulated drug delivery, which requires a certain time period after HIFU stimulation for the cell death to be experimentally measured, can be predicted by MRI shortly after HIFU stimulation based on the loss of viability versus R₂ plot. For example, from the observed change in R₂ value from 3.2 s⁻¹ before HIFU-stimulated release to 4.2 s⁻¹ after release, we may predict that there will be around 67% reduction in cell viability at 18 h after HIFU stimulation.

The present example illustrates a theranostics approach that uses MRI to characterize the MRgHIFU-stimulated drug release from a core-shell nanoparticle in vitro. Further, the present example illustrates that adjusting HIFU exposure times or power levels, controlled drug release and therapeutic efficacy (loss of cell viability) can be achieved, both of which were associated with MRI R₂ (1/T₂). Ideally, such characterization of MRgHIFU-stimulated drug release via MRI may eventually be applied for drug dose painting. For example, physicians may characterize and control the amount of drug release stimulated by HIFU during the treatment and predict therapeutic efficacy in patients. The ensuing doses may then be tuned by adjusting HIFU parameters (such as power levels or exposure times) in order to achieve a desired drug dosage in the therapeutic window. Another suitable application may be to assess whether the HIFU stimulation is precisely pin-pointed to the target site in order to stimulate drug release effectively. The present disclosure may provide an approach to achieve precision medicine, which includes administering drugs not only to the targeted diseased tissues at the right timing in patients, but also with the accurate drug doses.

### Superparamagnetic Core and Silica Shell MSNs for Theranostic Agent Delivery

MSNs with a superparamagnetic iron oxide nanoparticle SPION core and capping agents that control the active agents stored within the pores of the MSN offer several advantages. First, the superparamagnetic core makes the location of the MSNs imageable by T₂ and T₂* MRI. The core includes ˜20 nm diameter spheres of Fe₃O₄, which allows customization of both the size of the particle (by the synthesis conditions) and its composition (by doping or structural design features). Second, the internal porosity of the silica shell (˜2 nm diameter pores in a ˜30 nm thick shell) makes it possible to simultaneously carry large payloads of therapeutic agents (e.g., Doxorubicin, DOX) and/or MRI agents (e.g. T₁-shortening Gd chelates).

The MSNs are optimized to achieve high T₁, T₂ and T₂* MRI contrast enhancement by engineering the core and the uptake capacity of the imaging agent (e.g., Gd-DTPA). Different doped iron oxide nanoparticles are used to make the core. Different metal doped iron oxide nanoparticles with higher saturation magnetizations (>100 emu/g) than that of iron oxide nanoparticles (˜80 emu/g) were synthesized by a thermal decomposition method. Iron(III) acetylacetonate (Fe(acac)₃) was used as the iron precursor, 1,2-dodecandiol was used as a reducing agent, and oleic acid, and oleylamine were used as capping agents. Mn(acac)₂, Zn(acac)₂, or Co(acac)₂ were used as the precursors to obtain manganese, zinc, or cobalt doped iron oxide nanoparticles (i.e. MnFe₂O₄, Zn_{0.4}Fe_{2.6}O₄, or CoFe₂O₄), respectively. All the chemicals were dissolved and refluxed in benzyl ether at 298° C. The sizes of the first generation of the metal doped iron oxide nanoparticles were 6 nm in diameter.

Larger nanoparticles (˜9 nm and ˜11 nm) were obtained by growing metal doped iron oxide on the first generation or even the second generation of metal doped iron oxide nanoparticles. Larger metal doped iron oxide nanoparticles have a higher saturation magnetization, which is able to generate higher T₂ and T₂* contrast enhancement. The various types of metal doped iron oxide nanoparticles were able to be encapsulated in a mesoporous silica shell or a hollow mesoporous silica shell to form a core-shell structure which is able to load and release cargo (gadolinium complex, or anticancer drug) upon being triggered.

### Core-Shell MSNs to Deliver Drugs:

When the loaded active agents (e.g., drugs) are released from the pores, there will be a T₂ (or T₂*) change. The amount of drugs released can be quantitatively determined from the degree of T₂ (or T₂*) change.

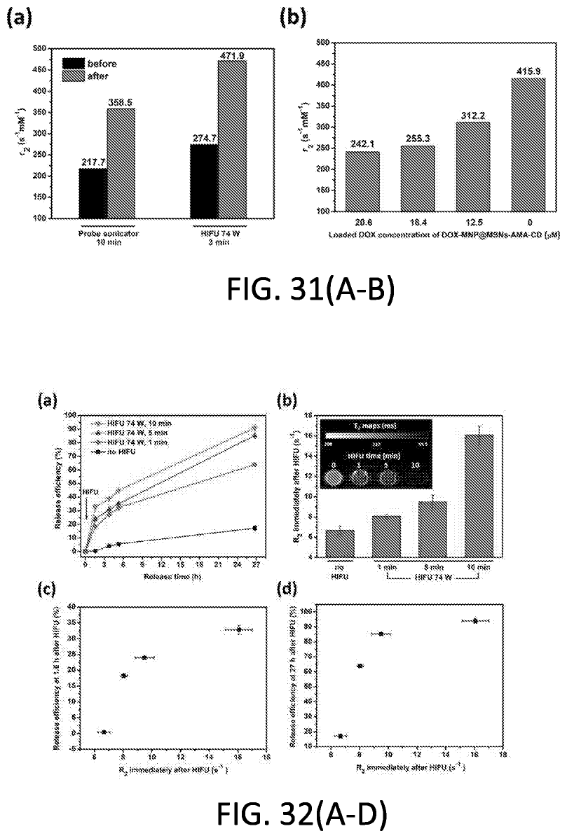

In this embodiment, a platform based on core-shell MSNs that are capped with a capping system containing aliphatic azo molecules (e.g. 4,4′-azobis(4-cyanovaleric acid). This aliphatic azo molecule possesses both thermal- and ultrasound-responsiveness. The capping system also contain bulky molecules (e.g. adamantane and cyclodextrin complexation) that are conjugated with the azo molecules and act as gatekeepers to control the cargo release from MSNs. To test the ultrasound responsiveness of this system, fluorescein was chosen as a model drug, loaded into nanoparticles (see below detail for loading procedure) and then capped with the capping system. Then, the release of fluorescein was triggered by using a probe sonicator. The cleavage of azo bonds makes the bulky gatekeeper leave from nanoparticle surface and the loaded fluorescein could be released. The release of fluorescein can be implied from the decrease in T₂ using MRI measurement. This T₂ change can be explained as more water molecules are able to diffuse into pores and got closer to the cores, as shown in FIG. 31.

### Using Core-Shell MSNs to Deliver Drugs:

When the loaded drugs are released from the pores, there will be a T₂ (or T₂*) change. As shown in FIG. 32, the amount of drugs released can be quantitatively determined from the degree of T₂ (or T₂*) change. HIFU stimulation was also applied to the nanoparticles, and again the decrease in T₂ was observed, which implies the release of fluorescein form the core-shell MSNs.

### Core-Shell MSNs to Co-Deliver Drugs and T₁ Contrast Agents:

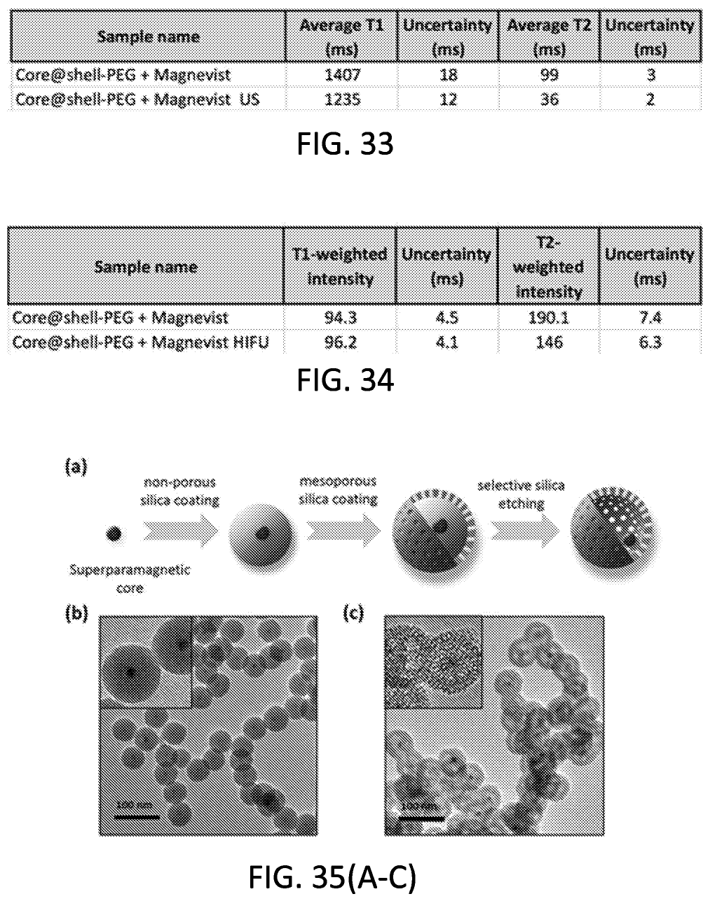

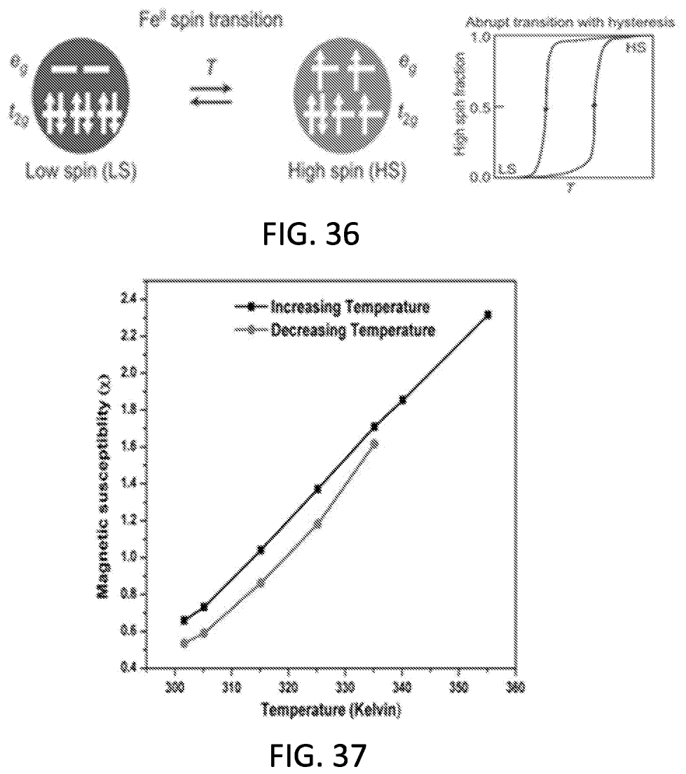

When the co-loaded drugs and T₁ contrast agents are released from the pores, there will be both T₁ and T₂ change. The amount of drugs released can be quantitatively determined from the degree of both T₁ and T₂ change. In one embodiment, the core-shell MSNs capped with the PEG capping system showed the decrease in T₂ after stimulated with the probe sonicator with the help of T₁ contrast agents, as shown in FIG. 33. This implies that when drugs are loaded in this particle, the release of drugs can be qualitatively and/or quantitatively determined from the degree of T₂ (or T₂*) change. HIFU stimulation was also applied to these nanoparticles. As shown in FIG. 36, the observed increase in T₁ weighted intensity and decrease in T₂ weighted intensity implies the release of fluorescein from the core-shell MSNs as described above.

### Hollow Core Mesoporous Silica Shell (Encapsulating an Iron Oxide Particle):

We also developed mesoporous silica nanoparticles with a hollow core that contains a small iron oxide particle (HMSNs) FIG. 35A. The superparamagnetic nanoparticle (˜11 nm) was coated by a non-porous silica shell FIG. 35B, whose thickness was adjusted by different weight ratio of tetraethyl orthosilicate (silica precursor) to superparamagnetic core. Then, a mesoporous silica shell (˜3 nm diameter pores in a ˜10 nm shell) was grown on the non-porous silica layer which was then selectively etched in a basic solution. The resulting hollow core-shell nanoparticles (˜60 nm) with a mesoporous silica shell and a superparamagnetic oxide particle in the hollow interior, FIG. 35C possess a larger pore volume (>1 cc/g) for cargo (e.g. gadolinium complex, or anticancer drug) loading. The porous shell thickness could be adjusted by adding different amount of tetraethyl orthosilicate during the synthesis. Ultrasound or HIFU responsive capping agents were conjugated to the surface to control the cargo release upon being stimulated.

### Diels-Alder Reactions:

In some embodiments a thermally reversible cycloaddition reaction (e.g., a Diels Alder reaction) is utilized to construct a capping agent that can trap cargo molecules inside the pores of MSNs. The conjugation between dienophile and diene-included bulky adamantane and cyclodextrin complex forming the basis of the capping agent that effectively block the pore opening of core@shell nanoparticles. The core is zinc, manganese-doped iron oxide (Zn_{0.4}Mn_{0.6}Fe₂O₄) nanoparticle and the shell is mesoporous silica. The cycloreversion can be triggered by an externally triggered ultrasound or HIFU resulting in heating and thus in the detachment of the cap from the pore openings and cargo release. This concept of a molecular nanocap based on a retro-Diels Alder reaction activated through the ultrasound or HIFU-induced heating adds to the toolbox of externally controllable, thermally triggered nanovalves. Actuation through HIFU has the advantage of pinpoint delivery and non-invasiveness, making these nanovalves useful candidates for applications in drug and/or imaging agent delivery.

Synthesis of Zinc and Manganese Doped-Iron Oxide Nanoparticle (Zn_{0.4}Mn_{0.6}Fe₂O₄):

Zinc and manganese doped iron oxide nanoparticles were synthesized following a thermal decomposition process as previously described. In brief, 0.353 g (1.00 mmol) Fe(acac)₃, 30.0 mg (0.220 mmol) ZnCl₂ and 63.3 mg (0.320 mmol) MnCl₂ were placed in a 50 mL three neck round bottom flask equipped with a reflux condenser under nitrogen atmosphere. 2.00 mL oleic acid, 4.00 mL oleylamine and 2.06 mL octylether were added and the reaction mixture was heated to 300° C. (SiC bath) for 1 h. The reaction mixture was cooled to room temperature and absolute ethanol was added. The resulting nanoparticles were washed three times with a mixture of chloroform and ethanol (1:10) by centrifugation (10 min, 26892 rcf) and finally redispersed in 10 mL of chloroform.

Synthesis of Zn_{0.4}Mn_{0.6}Fe₂O₄@MSN Core@Shell Nanoparticle:

Prior to the sol-gel reaction, the Zn_{0.4}Mn_{0.6}Fe₂O₄ were transferred from the organic phase to the aqueous phase. 4.285 mL of a 7 mg/mL SPION dispersion in CHCl₃ (corresponding to 30 mg of SPIONs) were placed in a polypropylene reactor. 21.7 g H₂O and 2.41 mL of aqueous CTAC solution (25 wt %) was added, generating a second phase. The mixture was sonicated for 15 min (60% of continuous power (250 W), frequency 20 KHz) using a probe sonicator and subsequently the chloroform was evaporated at elevated temperature (70° C.) for 2 h. After a second sonication step lasting 15 min, the mixture was added to 14.3 g TEA and stirred (1000 rpm) at 60° C. The silica source TEOS (10 times 155 μL, 692 μmol) was added 8 stepwise every 10 min over a total time period of 90 min at constant temperature of 60° C. The synthesis mixture was stirred at 1000 rpm at room temperature for 12 h. After addition of ethanol (100 mL), the SPION@MSNs were separated by centrifugation (43.146 rcf for 20 min) and redispersed in ethanol. The template extraction was performed twice by heating the SPION@MSN suspension under reflux at 90° C. (oil bath) for 45 min in an ethanolic solution (100 mL) containing ammonium nitrate (2 g). The SPION@MSNs were collected by centrifugation and washed with ethanol after each extraction step. The resulting nanoparticles were stored in an ethanolic solution.

Synthesis of Zn_{0.4}Mn_{0.6}Fe₂O₄@MSN-Mal:

20 mg of the unfunctionalized Zn_{0.4}Mn_{0.6}Fe₂O₄@MSN nanoparticles were washed 2× with toluene (2×1.5 mL), redispersed in 10 mL of dry toluene and stirred in aflame-dried 25 mL round bottom flask under nitrogen. Then, 40 μL of N-((3-triethoxysilyl)propyl)maleimide was added, and the resulting mixture was heated to reflux overnight. The nanoparticles (Zn_{0.4}Mn_{0.6}Fe₂O₄@MSN-Mal) were collected by centrifugation (5 min at 16873 rcf), washed 2× with toluene (2×1.5 mL) and redispersed in 2.5 mL of toluene.

Synthesis of Zn_{0.4}Mn_{0.6}Fe₂O₄@MSN-DA:

20 mg of Zn_{0.4}Mn_{0.6}Fe₂O₄@MSN-Mal in 2.5 mL of toluene were stirred in a glass vial together with 80 mg of N-(furan-2-ylmethyl)adamantane-1-carboxamide for 3 days at 40° C. The nanoparticles were collected by centrifugation in a cooled centrifuge (5 min at 20817 rcf and 18° C.), and washed 2× with toluene (2×1.5 mL), 2× with ethanol (2×1.5 mL) and 2× with water (2×1.5 mL).

Synthesis of Zn_{0.4}Mn_{0.6}Fe₂O₄@MSN-CD:

For loading the model drugs or imaging agents into the nanoparticles, 1 mg of sample SPION@MSN-DA were dispersed in 1 mL of an aqueous fluorescein solution (1 mM) or Magnevist solution and kept on a shaker over night at room temperature. For capping, 15 mg of beta-cyclodextrin was added to the solution, and shaking was continued for 1 d at room temperature. The nanoparticles were then collected by centrifugation in a cooled centrifuge (5 min at 20817 rcf and 18° C.), washed 5× with water (5×1.5 mL), and redispersed in 250 μL water.

### Azo-PEG:

In some embodiments, an azo-functionalized polymer (Azo-PEG) as a coating to block the pore opening of core@shell nanoparticles, preventing drug leakage at body temperature. Azo-PEG is thermoresponsive allowing the drug to release within a narrow temperature range that is biologically relevant. Azo-PEG responds to temperature by the breaking of covalent bonds, which makes it the first example of a thermodegradable polymer to trigger the drug release. The heat generated by ultrasound or HIFU can cleave the thermosensitive azo and pinpoint the release of the cargo in a high spatial control manner. Finally, this approach exhibits no cytotoxicity towards fibroblasts, demonstrating its safety. These new azo functionalized PEGcoated silica nanoparticles represent highly attractive and promising candidates to deliver active therapeutics and will be of great interest for nanomedicine applications such as in cancer therapy.

### Synthesis of Azo-PEG:

4,4′-azobis(4-cyanovaleric acid) (4.0 g, 14.29 mmol) was dissolved in 60 mL of dichloromethane. The solution was cooled to 0° C. in an ice bath to prevent cleavage of the azo bond. The carboxylic acid group was pre-activated with DIEA (3.7 mL, 21.42 mmol), HOBt (2.37 g, 17.85 mmol) and EDC.HCl (3.42 g, 17.85 mmol) for 30 min under argon atmosphere. Afterwards, NH₂-PEG-NH₂ (5.36 g, 3.57 mmol) was added. The mixture was stirred for another 4 h at room temperature under argon atmosphere. The resultant mixture was diluted in dichloromethane and washed with brine (3×200 mL). The combined organic layers were dried with magnesium sulfate, and then concentrated by rotary evaporation. The polymer was precipitated in cold diethylether and collected by filtration. Finally, the polymer was dialyzed overnight against deionized water (MWCO=1000 Da) at 4° C. and lyophilized.

Synthesis of Fe₃O₄@SiO₂ Core@Shell Nanoparticle

Core-shell Fe₃O₄@SiO₂ mesoporous nanoparticles were obtained through a two-step process. First 1 g of a chloroform dispersion of Fe₃O₄ nanoparticles (2.5 mg L⁻¹) was added to a 10 mL water solution containing 30 mg of cetrimonium bromide. The mixture was then sonicated for 10 min to allow a homogeneous dispersion of the organic solvent in the water phase after which the resulting solution was stirred at 85° C. to allow the chloroform to evaporate (10 min). Once the solution became clear the flask was sonicated for another minute to ensure a good dispersion of the Fe₃O₄ nanoparticles. After another 10 minutes at 85° C., 12.5 mg of arginine was added and finally 100 μL of tetraethyl orthosilicate were added rapidly. In order to obtain functionalized core-shell nanoparticles, (3-aminopropyl)triethoxysilane (APTES) was added to the mixture after 3 h leading to nanoparticles with amine group on the surface. The surfactant was extracting by mixing an ethanolic dispersion of the nanoparticle with ethanol and ammonium nitrate (6 g L⁻¹).

Polymer Grafting to Amine-Modified Core-Shell Fe₃O₄@SiO₂ Mesoporous Nanoparticles (MSN):

The polymers (Azo-PEG and PEG-COOH) were attached to the surface of the particles by standard coupling reaction between the carboxylic groups of the polymers and the amines at the surface of the particles. First the polymers (50 mg) were activated in water (1 mL) after mixing sequentially with DIEA (12 μL), HOBt (6 mg) and EDC.HCl (7 mg). Then the activated polymer solution was mixed with the nanoparticles (10 mg in 0.75 mL water) and let to react overnight at room temperature. During the first 15 min the reaction took place in an ice bath to avoid any local heating that may cause the degradation of the polymer. The grafting density of polymer was determined by thermogravimetric analysis. To account for the decomposition of the APTES modification, the final weight loss was determined by subtracting the weight loss of amine-modified nanoparticles (MSN) from the weight loss of the polymer-grafted nanoparticles (MSN-PEG or MSN-Azo-PEG). It was assumed the difference in weight loss was only from the attached polymer. The grafting density was estimated to be 7% and 13% for MSN-PEG and MSN-Azo-PEG respectively.

### Example 6: Exemplary Materials, Methods of Fabrication, and Characterization of Iron(II) Particles and Complexes as Described Herein

### Stimuli-Modulated Changes in Magnetization:

Instead of changing the access of the solvent molecules to the paramagnetic ion, the T₁ relaxivity of molecular and nano-crystalline contrast agents can be modulated by reversibly switching the spin state of specific molecules. For example, iron(II) complexes were developed that undergo photo or heat induced “spin-crossover” between S=0 (diamagnetic) and S=2 (paramagnetic) states (e.g., FIG. 36). This process is present in many six coordinate iron(II) complexes. The large paramagnetic susceptibility change will result in a large contrast change.

The temperature increase to change the spin state can be generated by switching on HIFU; switching off the HIFU allows the temperature to decrease back to ambient temperature. This system is reversible.

The compound that we used to demonstrate the above effects was Fe(Me-bik)₃](BF₄)₂.25H₂O. It has an octahedral structure with an octahedral {N6} coordination polyhedron. At low temperature the compound is in its diamagnetic low-spin (LS) configuration (t₂ g⁶, S=0) and is converted into a paramagnetic high-spin (HS) electron configuration (t₂ g⁴ e_{g}², S=2) at higher temperatures. The temperature dependence of the magnetic susceptibility is shown in FIG. 37.

T₁ and T₂ Values Measured Using NMR:

The T₁ values as a function of temperature were measured by NMR as shown in Table 9 below.

| Conc. | T₁ at 23° C. | T₁ at 40° C. | T₁ at 60° C. |
|---|---|---|---|
| 8 mg/mL | 2.7413 | 3.434 | 4.172 |
| 4 mg/mL | 2.7392 | 3.4784 | 4.2761 |
| 2 mg/mL | 2.8944 | 3.9614 | 4.9954 |
| 1 mg/mL | 2.9433 | 4.2093 | 5.3784 |
| water | 2.96 | 4.9147 | 7.1101 |

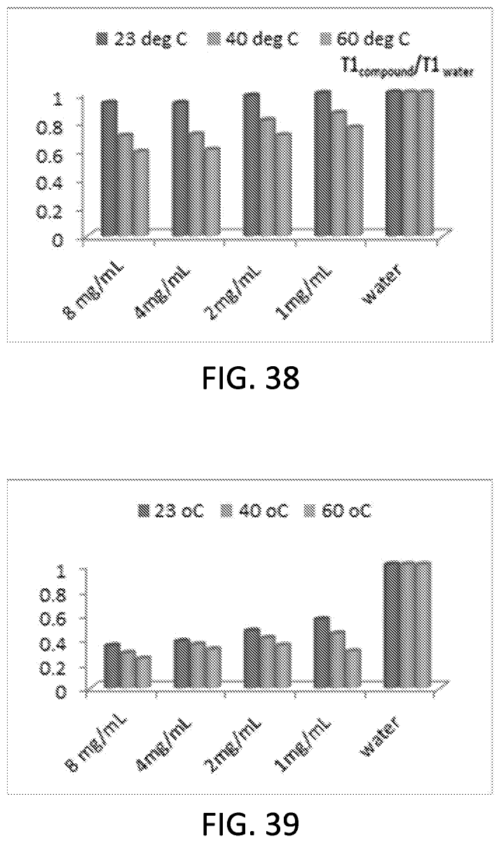

The Tt values for water increases with the rise in temperature and thus the overall T₁ values for the aqueous Fe(II) solution increases with rise in temperature. To account for the effect of the increases of T₁ of water with rise in temperature, the effect of the iron compound relative to that of water was calculated. The T₁(solution)/T₁(water) ratio for the different concentrations at different temperatures are shown in FIG. 38. The relative T₁ values shows the expected decrease with rise in temperature and corresponds with the fact that the magnetic moment increases with rise in temperature. Similarly, the relative T₂ values were recorded and the expected decrease is shown in FIG. 39.

### MRI Contrast Changes:

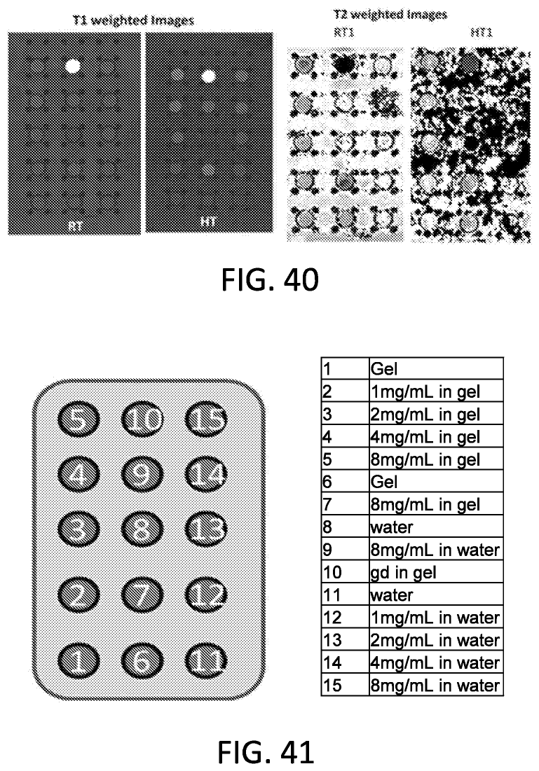

The samples were first placed in a water bath at room temperature and images were obtained. Then the same samples were placed in hot water bath (70° C.) and images were recorded again. The locations of the samples are defined in FIG. 41 and the MRI images are shown in FIG. 40. The darker T₁-weighted images at high temperature are a result of the presence of the iron complex in its high spin state. The relative T₁ and T₂ values with respect to gel or water shows decrease with rise in temperature in accordance with the measurements by NMR.

### Stimuli-Modulated Changes in Tumbling Rate of Ultrasmall Iron Oxide Nanoparticles:

Ultrasmall iron oxide nanoparticles (USIONs) (<4 nm) were synthesized by a thermal decomposition method. Iron(III) acetylacetonate was used as the iron precursor, 1,2-dodecandiol was used as a reducing agent, and oleic acid, and oleylamine were used as capping agents. All the chemicals were dissolved and refluxed in phenyl ether at 260° C. The average diameters of the USIONs were controlled by the reaction time.

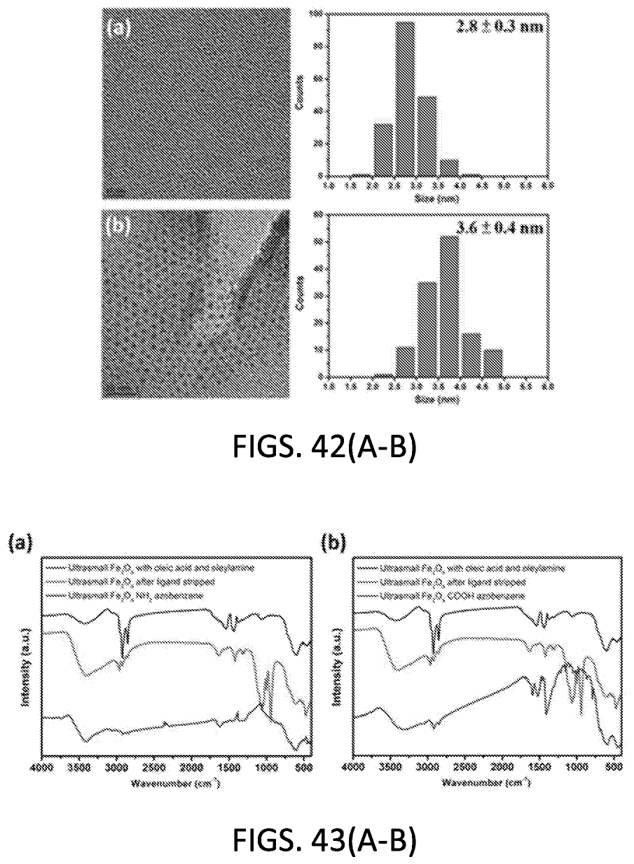

For example, USIONs with an average diameters of 2.8±0.3 nm, and 3.6±0.4 nm (FIG. 42) were synthesized by refluxing the reaction solution for 15 min and 30 min, respectively. To generate the photo-modulated changes in tumbling rate of the USIONs resulting in the T₁-weighted image changes, the surface capping agents were then replaced by aminoazobenzene or carboxyazobenzene with the property of photo-stimulated configuration change between cis and trans isomers. The surface capping agents (oleic acid and oleylamine) of USIONs were firstly stripped by nitrosyl tetrafluoroborate (NOBF₄) to get capping agents free USIONs. Aminoazobenzene or carboxyazobenzene (FIG. 43) molecules were then attached to the surface of USIONs.

Before the light illumination, the azobenzene molecules on the surface of USIONs displayed trans configuration, which made the whole nanoparticles have a slower tumbling rate and resulted in stronger T₁ contrast enhancement. On the contrary, when the particles were stimulated with light, the azobenzene molecules showed cis configuration, which made the whole nanoparticles have a faster tumbling rate and lead to less T₁-weighted image contrast enhancement. The innovative strategy we designed demonstrated that the T₁-weighted image contrast enhancement was modulated by the light-stimulated isomerization of azobenzene molecules on the USIONs.

### Exemplary Figures and Data Corresponding to Iron(II) Moieties, Chelates, Nanoparticles as Described Herein:

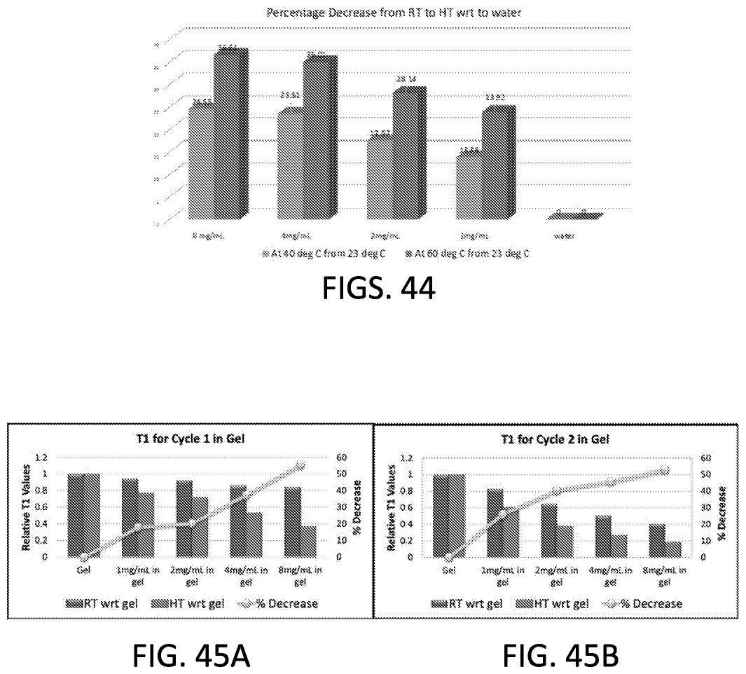

The percentage decrease of T₁ and T₂ values of the iron(II) complex relative to water decreased significantly with high temperature. FIG. 44 is a graph of the percentage decrease of T₁ values of different concentrations iron(II) complexes with respect to water at high temperatures compared to room temperature using NMR spectroscopy.

Reversibility of the system is checked using both MRI spectroscopy in both gel and water. In both media the particles showed reversibility as checked by two cycles temperature modulations. FIGS. 45A-B are graphs of the percentage decrease of T₁ values of iron(II) complexes of different concentrations with respect to gel at high temperatures compared to room temperature using MRI during cycle land cycle 2, respectively.

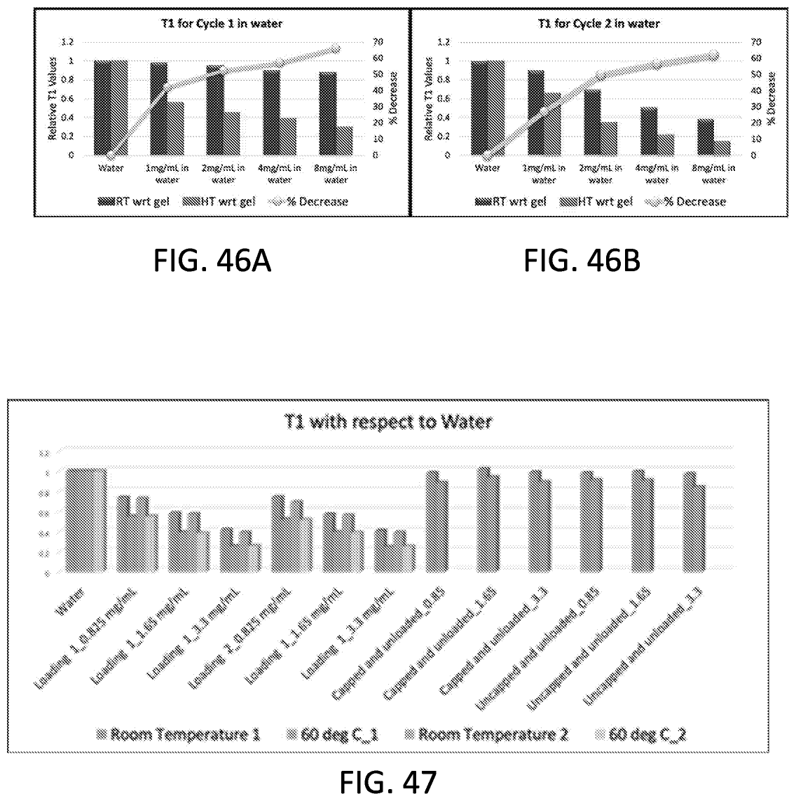

FIGS. 46A-13 are graphs of the percentage decrease of T₁ values of iron(II) complexes of different concentrations with respect to water at high temperatures compared to room temperature using MRI. FIG. 46A is during cycle 1. FIG. 46B is during cycle. To improve the biocompatibility, these spin crossover particles are loaded within the phosphonate-nanoparticles and then capped. The loading of the particles were done using two different protocols as labelled by loading 1 and loading 2.

The percentage decrease of T₁ values of iron(II) complexes with respect to gel at high temperatures compared to room temperature shows a greater enhancement when they are loaded within the phosphonate-nanoparticles as compared to the bare iron(II) complex. The reversibility of the complex within the nanoparticles is also analyzed by both NMR and MRI. The change in T₁ values with change in temperature is due to loaded complex within the nanoparticles and not due to particles itself. At room temperature the T₁ values for loaded iron (II) complexes are much lower than gel. A small amount of paramagnetic impurity can be present along with the diamagnetic iron(II) complex. There is no leakage of iron(II) complex at room temperature as well as at high temperature as checked by UV-vis absorbance spectroscopy.

FIG. 47 is a graph of T₁ values of different concentrations of loaded iron(II) complexes within phosphonate-nanoparticles, unloaded phosphonate-nanoparticles (capped and uncapped) with respect to gel at high temperatures compared to room temperature using NMR spectroscopy.

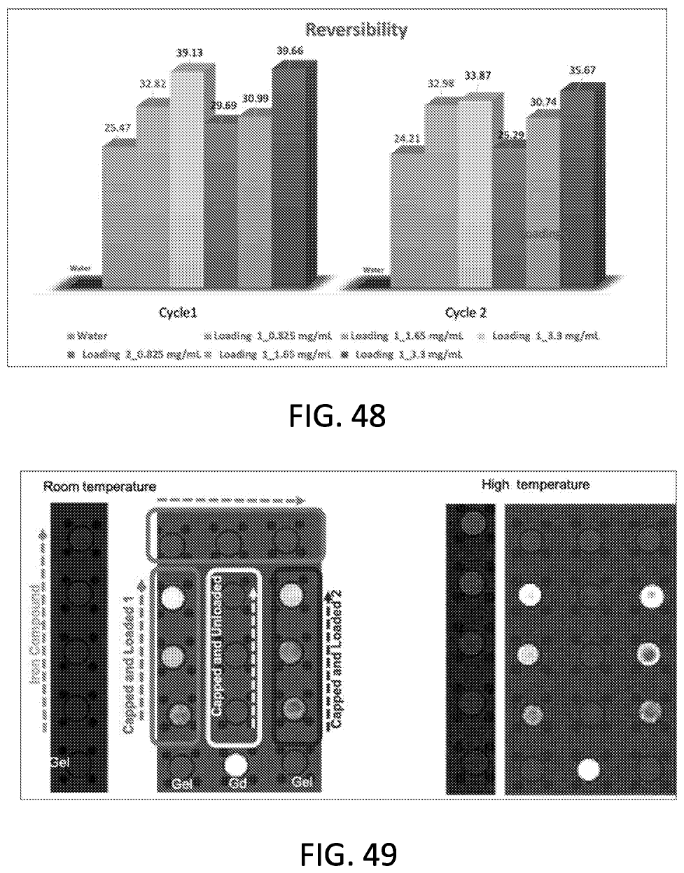

FIG. 48 is the percentage decrease of T₁ values of different concentrations of loaded iron(II) complexes within phosphonate-nanoparticles, unloaded phosphonate-nanoparticles (capped and uncapped) with respect to gel at high temperatures compared to room temperature using NMR spectroscopy. Reversibility of the systems are shown by performing cycle 1 and cycle 2.

FIG. 49 are T₁ weighted images of different concentrations of iron(II) complex, loaded iron(II) complexes within phosphonate-nanoparticles, unloaded phosphonate-nanoparticles (capped and uncapped) with respect to gel at high temperature compared to room temperature using MRI.

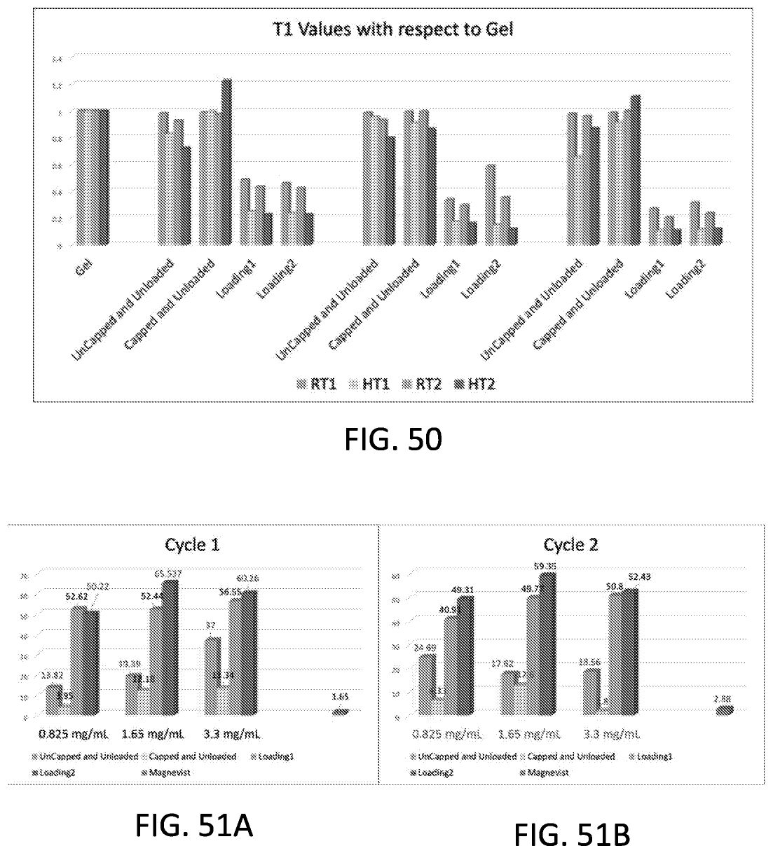

FIG. 50 is a graph of T₁ values of different concentrations of loaded iron(II) complexes within phosphonate-nanoparticles, unloaded phosphonate-nanoparticles (capped and uncapped) with respect to gel at high temperatures compared to room temperature using MRI.

FIGS. 51(A-B) are graphs of the percentage decrease of T₁ values of different concentrations of loaded iron(II) complexes within phosphonate-nanoparticles, unloaded phosphonate-nanoparticles (capped and uncapped) with respect to gel at high temperatures compared to room temperature using MRI. Reversibility of the systems are shown by performing cycle 1 and cycle 2.

The present disclosure has described one or more preferred embodiments, and it should be appreciated that many equivalents, alternatives, variations, and modifications, aside from those expressly stated, are possible and within the scope of the invention.

1. A stimuli-responsive composition comprising:
a silica particle having an outer surface and a plurality of pores that are sized to receive one or more active agent therein;
a plurality of capping agents coupled to the outer surface and arranged to cover at least a fraction of the plurality of pores, the capping agents having a first physical-chemical state that prevents the active agents from being released from at least a portion of the plurality of pores and a second physical-chemical state that allows the passage of the active agents from the plurality of pores,
wherein the capping agent is characterized as having a structure that is transformable from the first physical-chemical state to the second physical-chemical state in response to an external stimulus applied to the capping agents in an effective amount, and
wherein the capping agents are selected from one or more of:
a polymer having a polyether backbone;
a compound having an alkyl-azo moiety positioned along the length of the capping agent;
a macrocyclic molecule that is coupled to the silica particle through a linking agent, wherein the macrocyclic molecule is non-covalently bound to the linking agent; and
a poloxamer covalently bonded to the outer surface of the silica particle.

2. The composition of claim 1, wherein the silica particle comprises a silica nanoparticle.

3. The composition of claim 1, wherein the silica particle comprises a dimension between 20 nm and 300 nm.

4. The composition of claim 2, wherein the silica nanoparticle includes a silica shell having a hollow chamber formed therein, wherein the hollow chamber is at least partially filled with one or more superparamagnetic particle.

5. The composition of claim 4, wherein the superparamagnetic particle comprises iron oxide or the silica shell is directly coupled to a single superparamagnetic particle.

6-8. (canceled)

9. The composition of claim 1, wherein the capping agent is characterized as having a structure that is at least one of reversibly transformable from the first physical-chemical state to the second physical-chemical state in response to an external stimulus or stimuli applied to the capping agent; or
irreversibly transformable from the first physical-chemical state to the second physical-chemical state in response to an external stimulus or stimuli applied to the capping agent.

10. (canceled)

11. The composition of claim 1, wherein the polymer having a polyether backbone has an average molar mass that ranges between 400 Da to 25,000 Da.

12. The composition of claim 1, wherein the weight fraction of the capping agent in the composition is between 8% and 35%.

13. The composition of claim 11, wherein the polymer comprises polyethylene glycol.

14. The composition of claim 1, wherein one end of the alkyl-azo moiety is covalently bound to the silica particle and another end to a capping group of sufficient size to prevent the release of active agents from within the pore of the silica particle.

15. The composition of claim 1, wherein the alkyl-azo moiety includes at least one of:
an alkyl chain that ranges between 1 to 6 carbon atoms: or
moiety comprises 4,4′-azobis(4-cyanovaleric acid).

16. (canceled)

17. The composition of claim 1, wherein the macrocyclic molecule comprises at least one of:
glycouril monomers; or
cucurbit[6]uril.

18. (canceled)

19. The composition of claim 1, wherein the active agent comprises at least one of
a therapeutic agent;
a contrast agent; or
is entrained within the capping agent on the outer surface of the silica particle.

20-21. (canceled)

22. The composition of claim 1, wherein the external stimulus is selected from the group consisting of ultrasound, light, heat, and electromagnetic energy.

23. A method of delivering an active agent to a region of interest in a subject, the method comprising:
(a) administering a stimuli-responsive composition to the region of interest of the subject, wherein the stimuli-responsive composition comprises silica particles having an outer surface and a plurality of pores that are sized to receive one or more active agent therein; and a plurality of capping agents coupled to the outer surface and arranged to cover at least a fraction of the plurality of pores, the capping agents having a first physical-chemical state that prevents the active agents from being released from at least a portion of the plurality of pores and a second physical-chemical state that allows the passage of the active agents from the plurality of pores;
(b) applying an external stimulus to the capping agents in an effective amount to transform the capping agents from the first physical-chemical state to the second physical-chemical state to allow the passage of the active agent to the region of interest in the subject, and
wherein the active agent comprises a therapeutic agent and the external stimulus is applied to the capping agent for a sufficient dosage or duration to induce a therapeutic effect in the subject; or
wherein the active agent comprises a contrast agent and the external stimulus is applied to the capping agent for a sufficient dosage or duration to improve the visibility of the region of interest in the subject during a medical imaging procedure.

24. The method of claim 23, wherein the external stimulus is at least one of:
selected from the group consisting of ultrasound, light, heat, and electromagnetic energy;
applied using an external stimulus activation system having an ultrasound generator and transducer that is configured to apply ultrasound to the region of interest in the subject; or
applied using an external stimulus activation system having a light source that is configured to apply light to the region of interest in the subject.

25-26. (canceled)

27. The method of claim 23 further comprising (c) acquiring magnetic resonance imaging data from the region of interest of the subject using a magnetic resonance imaging system, and (d) producing a magnetic resonance image of the region of interest.

28. The method of claim 27, wherein step (c) further includes acquiring a first set of magnetic resonance imaging data from the region of interest while the silica particle is in the first physical-chemical state and acquiring a second set of magnetic resonance imaging data while the silica particle is in the second physical-chemical state.

29. The method of claim 28 further comprising computing a signal change map based at least in part on the difference between the first set of magnetic resonance imaging data and the second set of magnetic resonance imaging data, and generating an image of the region of interest based at least in part on the values of the signal change map.

30. The method of claim 23, wherein the first physical-chemical state of the capping agent substantially shields the active agent from the solvent in the region of interest in the subject, and wherein the second physical-chemical state exposes the active agent to the solvent in the region of interest of the subject.

31. The method of claim 28, wherein the capping agent comprises at least one of:
a polymer having a polyether backbone or a poloxamer; or
a thermo-responsive polymer having a reversible hydrophobicity.

32. (canceled)

33. The method of claim 31, wherein the thermo-responsive polymer includes at least one of:
a lower critical solution temperature within physiological conditions; or
comprises poly(N-isopropylacrylamide).

34. (canceled)

35. A method for producing a magnetic resonance image in a region of interest of a subject with enhanced contrast and reduced background signal, the method comprising:
(a) administering a stimuli-responsive composition to a region of interest in the region of interest of the subject, wherein the stimuli-responsive composition comprises a plurality of particles having a structure that is transformable from a first state to a second state in response to an external stimulus applied in an effective amount, wherein the second state changes magnetic resonance imaging contrast within the region of interest relative to the first physical state;
(b) applying an external stimulus to at least a portion of the particles for a first duration to alter the particles from a first state to a second state;
(c) acquiring a first set of magnetic resonance imaging data from the region of interest during the first duration when the particles are in the second state;
(d) ceasing the application of the external stimulus for a second duration to allow the particles to transform from the second state to the first state;
(e) acquiring a second set of magnetic resonance imaging data from the region of interest during the second duration when the particles are in the first state;
(f) computing a signal change map from the region of interest having values indicating a difference between the first set of magnetic resonance imaging data and the second set of magnetic resonance imaging data; and
(g) generating an image based at least in part on the values from the signal change map.

36. The method of claim 35, wherein the signal change map comprises a magnetic resonance relaxation parameter.

37. (canceled)

38. The method of claim 35, wherein steps (b)-(e) are repeated over multiple acquisition cycles during periodic modulation by the external stimulus.

39. The method of claim 38, wherein the periodic modulation comprises a modulation frequency that ranges between 0.01 Hz to 50 Hz.

40.-41. (canceled)

42. The method of claim 35, wherein the particles comprise silica nanoparticles having an outer surface and a plurality of pores that are sized to receive one or more contrast agent therein.

43. The method of claim 42, wherein the silica nanoparticles further include a plurality of capping agents coupled to the outer surface and arranged to cover at least a fraction of the plurality of pores, the capping agents having a first physical-chemical state that prevents the contrast agents from interacting with the magnetic resonance imaging contrast within the region of interest and a second physical-chemical state that allows the interaction of contrast agents with the magnetic resonance imaging contrast within the region of interest, and wherein the capping agent is characterized as having a structure that is transformable from the first physical-chemical state to the second physical-chemical state in response to the external stimulus applied to the capping agents in an effective amount.

44. The method of claim 35, wherein the nanoparticles are characterized as having a structure that undergoes spin-crossover from a first electronic state to a second electronic state in response to an external stimulus applied in an effective amount, wherein the particle is diamagnetic in the first electronic state and paramagnetic in the second electronic state.

45. The method of claim 44, wherein the nanoparticles comprise at least one of:
an iron(II) moiety;
an iron(II) chelate; or
an iron(II) moiety within nanoparticles.

46.-47. (canceled)

48. The method of claim 35, wherein the external stimulus is selected from the group consisting of ultrasound, light, heat, and electromagnetic energy.
